SEQUENCE LISTING (26 sequences)

SEQ ID NO: 1 - protein, 122 aa, Artificial Sequence
  Description of Artificial Sequence Synthetic polypeptide
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSS

SEQ ID NO: 2 - protein, 110 aa, Artificial Sequence
  Description of Artificial Sequence Synthetic polypeptide
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVL

SEQ ID NO: 3 - protein, 452 aa, Artificial Sequence
  Description of Artificial Sequence Synthetic polypeptide
QVQLVESGGGLVKPGGSLRLSCAASGFTFSSYALSWVRQAPGKGLEWVGRIRSKIDGGTTDYAAPVKGRFTISRDDSKNTLYLQMNSLKTEDTAVYYCARDRSPSDSSAFAIWGQGTLVTVSSASTKGPSVFPLAPSSKSTSGGTAALGCLVDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 212 aa, Artificial Sequence
  Description of Artificial Sequence Synthetic polypeptide
DIELTQPPSVSVSPGQTASITCSGDNLGSQYVDWYQQKPGQAPVLVIYDDNDRPSGIPERFSGSNSGNTATLTISGTQAEDEADYYCQSWDSLSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 457 aa, Artificial Sequence
  Description of Artificial Sequence Synthetic polypeptide
EVQLVESGGGLVQPGGSLRLSCAASGFPFTSTGISWVRQAPGKGLEWVGRTHLGDGSTNYADSVKGRFTISADTSKNTAYLQMNSLRAEDTAVYYCARTYGIYDTYDMYTEYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Description of Artificial Sequence Synthetic polypeptide
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 107 aa, Artificial Sequence [sequence omitted]

SEQ ID NO: 8 - protein, 114 aa, Artificial Sequence [sequence omitted]

SEQ ID NO: 9 - protein, 5 aa, Artificial Sequence
  Description of Artificial Sequence Synthetic peptide
NYYIH

SEQ ID NO: 10 - protein, 17 aa, Artificial Sequence
  Description of Artificial Sequence Synthetic peptide
AIYPDNSDTTYSPSFQG

SEQ ID NO: 11 - protein, 4 aa, Artificial Sequence
  Description of Artificial Sequence Synthetic peptide
GADI

SEQ ID NO: 12 - protein, 12 aa, Artificial Sequence
  Description of Artificial Sequence Synthetic peptide
RASQDIDPYLSN

SEQ ID NO: 13 - protein, 7 aa, Artificial Sequence
  Description of Artificial Sequence Synthetic peptide
DASNLQS

SEQ ID NO: 14 - protein, 9 aa, Artificial Sequence
  Description of Artificial Sequence Synthetic peptide
QQTTSHPYT

SEQ ID NO: 15 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 16 - protein, 111 aa, Artificial Sequence
  Description of Artificial Sequence Synthetic polypeptide
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVLG

SEQ ID NO: 17 - protein, 114 aa, Artificial Sequence [sequence omitted]

SEQ ID NO: 18 - protein, 107 aa, Artificial Sequence
  Description of Artificial Sequence Synthetic polypeptide
DIQMTQSPSSLSASVGDRVTITCKASQGVSNDYAWYQQKPGKAPKLLIYSASYRYTGVPSRFSGSGSGTDFTFTISSLQPEDIATYYCQQHSTTPYTFGQGTKLEIK

SEQ ID NO: 19 - protein, 119 aa, Artificial Sequence
  Description of Artificial Sequence Synthetic polypeptide
QIQLVQSGPELKKPGETVKISCKASGYTFTDFGMNWMKQAPGKGFKWMGWINTSTGESTYADDFKGRFAFSLETSASTAYLQINNLKNEDMATYFCARNSYYGGSYGYWGQGTTLTVSS

SEQ ID NO: 20 - protein, 113 aa, Artificial Sequence
  Description of Artificial Sequence Synthetic polypeptide
DIVMSQSPSSLAVSVGEKVTMKCKSSQSLLYSSNQKNYLAWYQQKPGQSPKLLIYWASTRESGVPDRFTGSGSGTDFTLTISSVKAEDLAVYYCQQYYSYPWTFGGGTKLEIK

SEQ ID NO: 21 - protein, 127 aa, Artificial Sequence
  Description of Artificial Sequence Synthetic polypeptide
EVQLVESGGGLVQPGGSLRLSCAASGFPFTSTGISWVRQAPGKGLEWVGRTHLGDGSTNYADSVKGRFTISADTSKNTAYLQMNSLRAEDTAVYYCARTYGIYDTYDMYTEYVMDYWGQGTLVTVSS

SEQ ID NO: 22 - protein, 107 aa, Artificial Sequence [sequence omitted]

SEQ ID NO: 23 - protein, 123 aa, Artificial Sequence
  Description of Artificial Sequence Synthetic polypeptide
QVQLVESGGGLVKPGGSLRLSCAASGFTFSSYALSWVRQAPGKGLEWVGRIRSKIDGGTTDYAAPVKGRFTISRDDSKNTLYLQMNSLKTEDTAVYYCARDRSPSDSSAFAIWGQGTLVTVSS

SEQ ID NO: 24 - protein, 107 aa, Artificial Sequence
  Description of Artificial Sequence Synthetic polypeptide
DIELTQPPSVSVSPGQTASITCSGDNLGSQYVDWYQQKPGQAPVLVIYDDNDRPSGIPERFSGSNSGNTATLTISGTQAEDEADYYCQSWDSLSVVFGGGTKLTVLG

SEQ ID NO: 25 - protein, 113 aa, Artificial Sequence
  Description of Artificial Sequence Synthetic polypeptide
QVQLVQSGAEVKKPGESLKISCKGSGYSFTNYYIHWVRQMPGKGLEWMGAIYPDNSDTTYSPSFQGQVTISADKSISTAYLQWSSLKASDTAMYYCARGADIWGQGTLVTVSS

SEQ ID NO: 26 - protein, 108 aa, Artificial Sequence
  Description of Artificial Sequence Synthetic polypeptide
DIQMTQSPSSLSASVGDRVTITCRASQDIDPYLSNWYQQKPGKAPKLLIYDASNLQSGVPSRFSGSGSGTDFTLTISSLQPEDFATYYCQQTTSHPYTFGQGTKVEIK

(19) United States
(12) Patent Application Publication
(10) Pub. No.: US 2023/0295308 A1
(43) Pub. Date: Sep. 21, 2023

(54) ANTAGONISTIC BIPARATOPIC ANTIBODIES THAT SPECIFICALLY BIND FIBROBLAST GROWTH FACTOR RECEPTOR 2 AND METHODS OF USING SAME

(71) Applicants: The Broad Institute, Inc., Cambridge, MA (US); Dana-Farber Cancer Institute, Inc., Boston, MA (US)

(72) Inventors: Saireudee CHATURANTABUT, Cambridge, MA (US); William SELLERS, Cambridge, MA (US)

(73) Assignees: The Broad Institute, Inc., Cambridge, MA (US); Dana-Farber Cancer Institute, Inc., Boston, MA (US)

(21) Appl. No.: 18/008,108

(22) PCT Filed: Jun. 2, 2021
(86) PCT No.: PCT/US2021/035468

§ 371 Date: Dec. 2, 2022

Related U.S. Application Data
(60) Provisional application No. 63/033,975, filed on Jun. 3, 2020.

(51) Int. Cl. C07K 16/28; A61P 35/00; A61P 1/16

(52) U.S. Cl. CPC C07K 16/2863; A61P 35/00; A61P 1/16; C07K 2317/31; C07K 2317/565; C07K 2317/76; C07K 2317/92; A61K 2039/505

(57) ABSTRACT

Described and featured herein are antagonistic biparatopic antibodies that specifically bind and inhibit an FGF receptor (e.g., FGFR2) and methods of using such antibodies for the treatment of cancers, including Cholangiocarcinoma (CCAs).

### CROSS REFERENCE TO RELATED APPLICATIONS

This application is the U.S. National Stage Application, pursuant to 35 U.S.C. § 371 of PCT International Application No. PCT/US2021/035468, filed Jun. 2, 2021, designating the United States and published in English, which claims priority to and the benefit of U.S. Provisional Application No. 63/033,975, filed Jun. 3, 2020, the contents of each of which are incorporated by reference herein in their entirety.

### STATEMENT OF RIGHTS TO INVENTIONS MADE UNDER FEDERALLY SPONSORED RESEARCH

This invention was made with government support under Grant No. CA127003 awarded by the National Institutes of Health. The government has certain rights in the invention.

### SEQUENCE LISTING

This application contains a Sequence Listing that has been submitted electronically in ASCII format and is hereby incorporated by reference in its entirety. Said ASCII copy, created on Jul. 6, 2021, is named 167741_022601_PCT_SL.txt and is 37,595 bytes in size.

### BACKGROUND

Cholangiocarcinomas (CCAs) are aggressive tumors arising from the biliary tract with limited treatment options and poor overall survival. The fibroblast growth factor receptor (FGFR) pathway is involved in cellular processes required for cell survival and differentiation, and aberrant FGFR signaling can result in oncogenic changes. Recently, FGFR2 gene fusions have been found to be associated with CCAs. Accordingly, agents that inhibit FGFR are likely to be useful for the treatment of CCA.

The development of antagonistic antibodies represents a therapeutic strategy with significant clinical potential. However, challenges remain in achieving clinical efficacy. Bispecific and biparatopic antibodies represent an emerging class of drug molecules that enable unique mechanisms of action relative to their monospecific counterparts. A bispecific antibody is a single molecule that includes two Fab variable domains each of which binds a distinct antigen. Knobs-into-holes technology has been used to drive assembly of bispecific antibodies toward heterodimer formation. A biparatopic antibody is a molecule that includes two Fab variable domains, each of which binds a distinct epitope on a single antigen. Many bivalent antibodies act as agonists. Antagonistic biparatopic antibodies against the FGFR2 receptor would provide an important therapeutic for the treatment of CCA, and such agents are urgently required.

### SUMMARY

Featured herein are antagonistic biparatopic antibodies that specifically bind and inhibit an FGF receptor (e.g., FGFR2) and methods of using such antibodies for the treatment of cancers, including Cholangiocarcinomas (CCAs).

In one aspect, a polypeptide that specifically binds two epitopes in the extracellular domain of a fibroblast growth factor receptor 2 (FGFR2) is provided, where the polypeptide contains two antigen binding fragments of anti-FGFR2 antibodies. In one embodiment, the anti-FGFR2 antibodies are any one or more of M048-D01, GAL-FR23, 10164, 2B 1.3.12, GAL-FR21, and 12433.

In another aspect, a biparatopic antibody that specifically binds two epitopes in the extracellular domain of a fibroblast growth factor receptor 2 (FGFR2) is provided, where the biparatopic antibody contains antigen binding fragments of an antibody selected from any one or more of M048-D01, GAL-FR23, 10164, 2B 1.3.12, GAL-FR21, and 12433.

In another aspect, a method of inhibiting the proliferation or reducing survival of a neoplastic cell is provided, in which the method involves contacting the cell with an effective amount of the polypeptide or antibody of any previous aspect, thereby inhibiting proliferation or reducing viability. In one embodiment, the polypeptide or antibody induces cell death of the neoplastic cell. In another embodiment, the neoplastic cell is a cholangiocarcinoma (CCA) cell. In another embodiment, the cell is in vitro or in vivo.

In another aspect, a method of treating cancer in a subject is provided, in which the method involves administering to the subject an effective amount of the polypeptide or antibody of any previous aspect, thereby treating the cancer. In one embodiment, the neoplastic cell is a cholangiocarcinoma (CCA) cell.

In another aspect, a method of treating cholangiocarcinoma in a subject is provided, in which the method involves administering to the subject an effective amount of a biparatopic antibody containing antigen binding fragments of an antibody selected from the group consisting of M048-D01, GAL-FR23, 10164, 2B 1.3.12, GAL-FR21, or 12433. In an embodiment of the method, an effective amount of a biparatopic antibody comprising FGFR2 antigen binding fragments of antibody M048-D01 and antibody 12433; or antigen binding fragments of antibody GAL-FR21 and antibody 12433, or antigen binding fragments of HuGAL-FR21 and antibody 12433; or antigen binding fragments of antibody HuGAL-FR21 and antibody GAL-FR23; or antigen binding fragments of antibody GAL-FR23 and antibody 12433; or antigen binding fragments of antibody M048-D01 and antibody 12433; or antigen binding fragments of antibody 2B 1.3.12 and antibody 10164; or antigen binding fragments of antibody 2B 1.3.12 and antibody 12433; or antigen binding fragments of antibody GAL-FR23 and antibody 2B 1.3.12; or antigen binding fragments of antibody GAL-FR23 and antibody HuGAL-FR21; or antigen binding fragments of antibody GAL-FR23 and antibody 12433 is administered to the subject. In an embodiment, cells of the subject comprise and FGFR2 fusion. In an embodiment, the FGFR2 fusion is FGFR2-PHGDH or FGFR2-BICC1. In an embodiment of the method, the biparatopic antibody has a KD for binding to FGFR2 of from about 7.7E-09 to about 9.1E-10.

In another aspect, an isolated nucleic acid molecule that encodes the polypeptide or antibody of any previous aspect is provided.

In another aspect, a vector containing a nucleic acid molecule that encodes the polypeptide or antibody of any previous aspect is provided. In one embodiment, the vector is an expression vector. In another embodiment, the expression vector is a viral or non-viral expression vector. In another embodiment, the expression vector encodes an affinity tag or a detectable amino acid sequence operably linked to the polypeptide or antibody.

In another aspect, a host cell that contains the vector of any previous aspect is provided.

In another aspect, a pharmaceutical composition containing an effective amount of the polypeptide or antibody of any previous aspect, or fragments thereof, in a pharmaceutically acceptable excipient is provided.

In another aspect, a method of treating cholangiocarcinoma in a subject is provided, in which the method involves administering to the subject an effective amount of an antibody of any previous aspect and an effective amount of pemigatinib or NVP-BGJ398.

In various embodiments of any of the above aspects or any other aspect and/or embodiments thereof as delineated herein, the polypeptide or antibody contains one or more complementarity determining regions of the antibody. In various embodiments of any of the above aspects or any other aspect as delineated herein, the polypeptide or antibody contains a heavy chain variable domain (VH) or a light chain variable domain (VL). In various embodiments of any of the above aspects or any other aspect as delineated herein, the antibody or polypeptide specifically binds an FGFR2 signal peptide (SP) or immunoglobulin-like domains IgI, IgII or IgIII. In various embodiments of any of the above aspects or any other aspect as delineated herein, the antibody or polypeptide specifically binds an FGFR2 immunoglobulin-like domain IgI, IgII or IgIII. In particular embodiments of any of the above aspects, the antibody binds SP and IgI, SP and IgII, SP and IgIII, SP and IgII+IgIII, IgI and IgII, IgI and IgIII, IgI and IgII+IgIII, IgII and IgIII, IgII and IgII+IgIII, or IgIII and IgII+IgIII. In various embodiments of any of the above aspects or any other aspect as delineated herein, the antibody or polypeptide specifically binds two fragments of an FGFR2 immunoglobulin-like domain, where the fragments are derived from IgI and IgII, IgI and IgIII, or IgII and IgIII. In various embodiments of any of the above aspects or any other aspect as delineated herein, the antibody or polypeptide specifically binds two fragments of an FGFR2 immunoglobulin-like domain IgI, IgII, or IgIII. In various embodiments of any of the above aspects or any other aspect as delineated herein, the antibody or polypeptide binding blocks ligand binding to FGFR2. In various embodiments of any of the above aspects or any other aspect as delineated herein, the antibody or polypeptide binding reduces FGFR2 activity. In another embodiment, the antigen binding fragment has at least 85% amino acid sequence identity to the sequence of M048-D01, GAL-FR23, 10164, 2B 1.3.12, GAL-FR21, or 12433. In another embodiment, the antigen binding fragment has at least 90% amino acid sequence identity to the sequence of M048-D01, GAL-FR23, 10164, 2B 1.3.12, GAL-FR21, or 12433. In another embodiment, the antigen binding fragment has at least 95% amino acid sequence identity to the sequence of M048-D01, GAL-FR23, 10164, 2B 1.3.12, GAL-FR21, or 12433. In another embodiment, the antigen binding fragment contains or consists essentially of a complementarity determining region of M048-D01, GAL-FR23, 10164, 2B 1.3.12, GAL-FR21, or 12433. In another embodiment, the polypeptide comprises an affinity tag. In another embodiment, the polypeptide comprises a detectable amino acid sequence.

In other embodiments, the biparatopic antibody of the above-delineated aspects and/or embodiments thereof, comprises antigen binding fragments of antibody M048-D01 and antibody 12433; or antigen binding fragments of antibody GAL-FR21 and antibody 12433, or antigen binding fragments of HuGAL-FR21 and antibody 12433; or antigen binding fragments of antibody HuGAL-FR21 and antibody GAL-FR23; or antigen binding fragments of antibody GAL-FR23 and antibody 12433; or antigen binding fragments of antibody M048-D01 and antibody 12433; or antigen binding fragments of antibody 2B 1.3.12 and antibody 10164; or antigen binding fragments of antibody 2B 1.3.12 and antibody 12433; or antigen binding fragments of antibody GAL-FR23 and antibody 2B 1.3.12; or antigen binding fragments of antibody GAL-FR23 and antibody HuGAL-FR21; or antigen binding fragments of antibody GAL-FR23 and antibody 12433.

In other embodiments, the polypeptide or the biparatopic antibody of any of the above-delineated aspects and/or embodiments thereof, has a KD for binding to FGFR2 of from about 7.7E-09 to about 9.1E-10. In other embodiments, the polypeptide or the biparatopic antibody of the above-delineated aspects and/or embodiments thereof has a KD for binding to FGFR2 selected from about 1.3E-09, 7.7E-09, 2.5E-10, 3.7E-10, 3.9E-10, 4.2E-10, 5.0E-10, 5.3E-10, 6.8E-10, 7.7E-10, 8.7E-10, or 9.1E-10.

In other embodiments of the above delineated aspects or embodiments thereof, the biparatopic antibody comprising FGFR2 antigen binding fragments of antibody HuGAL-FR21 and antibody 12433; or antigen binding fragments of antibody HuGAL-FR21 and antibody GAL-FR23; or antigen binding fragments of antibody GAL-FR21 and antibody 12433; or antigen binding fragments of antibody GAL-FR23 and antibody 12433 inhibited growth of cells expressing or overexpressing an FGFR2 fusion. In an embodiment, the FGFR2 fusion is FGFR1-PHGDH. In other embodiments of the above-delineated aspects or embodiments thereof, the biparatopic antibody comprising FGFR2 antigen binding fragments of antibody 2B 1.3.12 and antibody 10164; or antigen binding fragments of antibody 2B 1.3.12 and antibody 12433; or antigen binding fragments of antibody GAL-FR23 and antibody 2B 1.3.12; or antigen binding fragments of antibody GAL-FR23 and antibody HuGAL-FR21; or antigen binding fragments of antibody GAL-FR23 and antibody 12433 inhibited growth of cells expressing or overexpressing an FGFR2 fusion. In an embodiment, the FGFR2 fusion is FGFR2-BICC1.

### Definitions

Unless defined otherwise, all technical and scientific terms used herein have the meaning commonly understood by a person skilled in the art to which the aspects and embodiments described herein belong. The following references provide one of skill with a general definition of many of the terms used in the described aspects and embodiments: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a small compound, protein, nucleic acid molecule, or fragment thereof. In various embodiments, agents (e.g., biparatopic antibodies and fragments thereof) that bind and antagonize an FGF receptor (e.g., FGFR2) are provided.

By “ameliorate” is meant decrease, suppress, attenuate, diminish, arrest, or stabilize the development or progression of a disease. Cholangiocarcinoma is one exemplary disease amenable to treatment using the biparatopic antibodies described herein.

As used herein, the term “antibody” (Ab) refers to an immunoglobulin molecule that specifically binds to, or is immunologically reactive with, a particular antigen, and includes polyclonal, monoclonal, genetically and molecularly engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments. Moreover, unless otherwise indicated, the term “monoclonal antibody” (mAb) is meant to include both intact molecules, as well as, antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less nonspecific tissue binding than an intact antibody (see, Wahl et al., J. Nucl. Med. 24:316, 1983; incorporated herein by reference). In one embodiment, an antibody is a biparatopic antibody. Exemplary antibodies A-F, which are defined below, are useful in the methods in the various aspects and embodiments described herein. Without intending to be limiting, bispecific antibodies provide the ability to recognize and bind to two different antigens or epitopes (antigen domains) simultaneously as a single molecule. Biparatopic antibodies, which constitute a subset of bispecific antibodies, comprise antigen binding sites (paratopes) that provide the ability to recognize and bind to two different epitopes or antigenic sites on the same target antigen. In an embodiment, the antigen binding domains of biparatopic antibodies recognize and bind unique, non-overlapping epitopes on the same target antigen, such as a receptor. In an embodiment, the receptor is an FGFR receptor, e.g., FGFR1, FGFR2, FGFR2 alpha IIIb, FGFR3, and FGFR4. Such antibodies are advantageous as beneficial therapeutic antibodies for use in the treatment of diseases, such as CCA.

By “M048-D01 polypeptide” (also referred to as Antibody A) is meant an antibody or antigen binding fragment thereof having at least about 85% amino acid sequence identity to an antibody sequence of M048-D01 as described in WO2013076186 that specifically binds FGFR2. In embodiments, the antibody or antigen binding fragment thereof has at least about 90%, 93%, 95%, 98%, 99% or greater amino acid sequence identity to an antibody sequence of M048-D01. Exemplary sequences for M048-D01 are provided below:

```
M048-D01 VH chain
(SEQ ID NO: 1)
Glu Val Gln Leu Leu Glu Ser Gly Gly Gly Leu Val
1 5 10
Gln Pro Gly Gly Ser Leu Arg Leu Ser Cys Ala Ala
                15 20
Ser Gly Phe Thr Phe Ser Ser Tyr Ala Met Ser Trp
25 30 35
Val Arg Gln Ala Pro Gly Lys Gly Leu Glu Trp Val
                        40 45
Ser Ala Ile Ser Gly Ser Gly Thr Ser Thr Tyr Tyr
        50 55 60
Ala Asp Ser Val Lys Gly Arg Phe Thr Ile Ser Arg
                                65 70
Asp Asn Ser Lys Asn Thr Leu Tyr Leu Gln Met Asn
                75 80
Ser Leu Arg Ala Glu Asp Thr Ala Val Tyr Tyr Cys
85 90 95
Ala Arg Val Arg Tyr Asn Trp Asn His Gly Asp Trp
                        100 105
Phe Asp Pro Trp Gly Gln Gly Thr Leu Val Thr Val
        110 115 120
Ser Ser
M048-D01 VL chain
SEQ ID NO: 32 of WO2013076186 is provided below.
(SEQ ID NO: 2)
Gln Ser Val Leu Thr Gln Pro Pro Ser Ala Ser Gly
1 5 10
Thr Pro Gly Gln Arg Val Thr Ile Ser Cys Ser Gly
                15 20
Ser Ser Ser Asn Ile Gly Asn Asn Tyr Val Ser Trp
25 30 35
Tyr Gln Gln Leu Pro Gly Thr Ala Pro Lys Leu Leu
                        40 45
Ile Tyr Glu Asn Tyr Asn Arg Pro Ala Gly Val Pro
        50 55 60
Asp Arg Phe Ser Gly Ser Lys Ser Gly Thr Ser Ala
                                65 70
Ser Leu Ala Ile Ser Gly Leu Arg Ser Glu Asp Glu
                75 80
Ala Asp Tyr Tyr Cys Ser Ser Trp Asp Asp Ser Leu
85 90 95
Asn Tyr Trp Val Phe Gly Gly Gly Thr Lys Leu Thr
                        100 105
Val Leu
        110
```

By “M048-D01 polynucleotide” is meant a nucleic acid molecule encoding an M048-D01 antibody.

By “GAL-FR23 polypeptide” or “FR23 polypeptide” (also referred to as Antibody B) is meant an antibody or antigen binding fragment thereof having at least about 85% amino acid sequence identity to the antibody produced by the hybridoma deposited at PTA-9408 on Aug. 12, 2008, under the Budapest Treaty as described in U.S. Pat. No. 9,382,324 that specifically binds FGFR2.

By “GAL-FR23 polynucleotide” is meant a is meant a nucleic acid molecule encoding an GAL-FR23 antibody.

By “10164 polypeptide” (also referred to as Antibody C) is meant an antibody or antigen binding fragment thereof having at least about 85% amino acid sequence identity to an antibody sequence of 10164 as described in U.S. Pat. No. 9,498,532 and in WO2014163714A2 that specifically binds FGFR2. In embodiments, the antibody or antigen binding fragment thereof has at least about 90%, 93%, 95%, 98%, 99% or greater amino acid sequence identity to an antibody sequence of 10164.

FGFR_10164
Heavy Chain
(SEQ ID NO: 3)
QVQLVESGGGLVKPGGSLRLSCAASG FTFSSYALSWVRQAPGKGLEWVG
RIRSKIDGGTTDYAAPVKGRFTISRDDSKNTLYLQMNSLKTEDTAVYYCA
RDRSPSDSSAFAIWGQGTLVTVSSASTKGPSVFPLAPSSKSTSGGTAALG
CLVDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKENWYVDGVEVHNAKTKPREE
QYNSTYRVVSVLTVLHQDWLNGKEYKCKVSWESNGQPENNYKTTPPVLDS
DGSFFLYSKLTVDKSRWQQGNVFSCSVMHEALHNHYTQKSLSLSPGK
Light chain
(SEQ ID NO: 4)
DIELTQPPSVSVSPGQTASITCSGDNLGSQYVDWYQQKPGQAPVLVIYDD
NDRPSGIPERFSGSNSGNTATLTISGTQAEDEADYYCQSWDSLSVVFGGG
TKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGS
TVEKTVAPTECS

By “10164 polynucleotide” is meant a nucleic acid molecule encoding an GAL-FR23 antibody.

By “2B 1.3.12 polypeptide” (also referred to as Antibody D) is meant an antibody or antigen binding fragment thereof having at least about 85% amino acid sequence identity to a sequence of 2B 1.3.12 as described in U.S. Pat. No. 10,208,120 that specifically binds FGFR2. In embodiments, the antibody or antigen binding fragment thereof has at least about 90%, 93%, 95%, 98%, 99% or greater amino acid sequence identity to an antibody sequence of 2B 1.3.12.

2B.1.3.12 heavy chain, amino acid
(SEQ ID NO: 5)
EVQLVESGGGLVQPGGSLRLSCAASGFPFTSTGISWVRQAPGKGLEWVGR
THLGDGSTNYADSVKGRFTISADTSKNTAYLQMNSLRAEDTAVYYCARTY
GIYDTYDMYTEYVMDYWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK
2B.1.3.12 light chain, amino acid
(SEQ ID NO: 6)
DIQMTQSPSSLSASVGDRVTITCRASQDVDTSLAWYKQKPGKAPKLLIYS
ASFLYSGVPSRFSGSGSGTDFTLTISSLQPEDFATYYCQQSTGHPQTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

By “2B 1.3.12 polynucleotide” is meant a is meant a nucleic acid molecule encoding an 2B 1.3.12 antibody.

By “HuGAL-FR21 polypeptide” or “FR21” (also referred to as Antibody E) is meant an antibody or antigen binding fragment thereof having at least about 85% amino acid sequence identity to an antibody sequence of GAL-FR21 as described in U.S. Pat. No. 9,382,324 that specifically binds FGFR2. In embodiments, the antibody or antigen binding fragment thereof has at least about 90%, 93%, 95%, 98%, 99% or greater amino acid sequence identity to an antibody sequence of GAL-FR21 or HuGal-Fr21. In one embodiment, the antibody GAL-FR21 comprises the monoclonal antibody (mAB) produced by the hybridoma deposited at the American Type Culture Collection, P.O. Box 1549 Manassas, Va. 20108, as PTA-9586 on Nov. 6, 2008 under the Budapest Treaty.

A light chain variable region of mAB HuGal-FR21
(SEQ ID NO: 7):
Asp Ile Gln Met Thr Gln Ser Pro Ser Ser Leu Ser
Ala Ser Val Gly Asp Arg Val Thr Ile Thr Cys Lys
Ala Ser Gln Gly Val Ser Asn Asp Val Ala Trp Tyr
Gln Gln Lys Pro Gly Lys Ala Pro Lys Leu Leu Ile
Tyr Ser Ala Ser Tyr Arg Tyr Thr Gly Val Pro Ser
Arg Phe Ser Gly Ser Gly Ser Gly Thr Asp Phe Thr
Phe Thr Ile Ser Ser Leu Gln Pro Glu Asp Ile Ala
Thr Tyr Tyr Cys Gln Gln His Ser Thr Thr Pro Tyr
Thr Phe Gly Gln Gly Thr Lys Leu Glu Ile Lys
A heavy chain variable region of mAB HuGal-FR21
(SEQ ID NO: 8)
Gln Val Gln Leu Val Gln Ser Gly Ala Glu Val Lys
Lys Pro Gly Ser Ser Val Lys Val Ser Cys Lys Ala
Ser Gly Tyr Ile Phe Thr Thr Tyr Asn Val His Trp
Val Arg Gln Ala Pro Gly Gln Gly Leu Glu Trp Ile
Gly Ser Ile Tyr Pro Asp Asn Gly Asp Thr Ser Tyr
Asn Gln Asn Phe Lys Gly Arg Ala Thr Ile Thr Ala
Asp Lys Ser Thr Ser Thr Ala Tyr Met Glu Leu Ser
Ser Leu Arg Ser Glu Asp Thr Ala Val Tyr Tyr Cys
Ala Arg Gly Asp Phe Ala Tyr Trp Gly Gln Gly Thr
Leu Val Thr Val Ser Ser

By “HuGAL-FR21 polynucleotide” is meant a is meant a nucleic acid molecule encoding an HuGAL-FR21 antibody.

By “12433 polypeptide” (also referred to as Antibody F) is meant an antibody or antigen binding fragment thereof having at least about 85% amino acid sequence identity to an antibody sequence of 12433 as described in US Patent Publication No. 20190345250 that specifically binds FGFR2. In embodiments, the antibody or antigen binding fragment thereof has at least about 90%, 93%, 95%, 98%, 99% or greater amino acid sequence identity to an antibody sequence of 12433. Antibody F, No. 12433, is described at Table 1 of US Patent Publication No. 20190345250. Antibody 12433 includes the following exemplary sequences:

(SEQ ID NO: 9)
HCDR1 NYYIH(Kabat);
(SEQ ID NO: 10)
HCDR2 AIYPDNSDTTYSPSFQG;
(SEQ ID NO: 11)
HCDR3 GADI;
(SEQ ID NO: 12)
LCDR1 RASQDIDPYLSN,
(SEQ ID NO: 13)
LCDR2 DASNLQS,
(SEQ ID NO: 14)
LCDR3 QQTTSHPYT

By “12433 polynucleotide” is meant a is meant a nucleic acid molecule encoding a 12433 antibody.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art. In some embodiments, antigen-binding fragments (e.g., .g., Fab′, F(ab′)₂, Fab, scFab, Fv, rlgG, and scFv fragments) of a biparatopic antibody, which are joined by a synthetic linker, are provided.

By “alteration” is meant a change (increase or decrease) in the structure, expression levels or activity of a gene or polypeptide as detected by standard art known methods such as those described herein. As used herein, an alteration includes a 10% change in expression or activity levels, a 25% change, a 40% change, and a 50% or greater change in expression or activity levels. In some embodiments, an alteration in a biparatopic antibody is a sequence alteration that enhances binding to a target protein, stability, expression, or activity. In another embodiment, an alteration involves a decrease in the activity of FGFR2, which is associated with binding of a biparatopic antibody described herein.

By “analog” is meant a molecule that is not identical, but that has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. In addition, analogs of biparatopic antibodies that retain or enhance the activity of the original antibody are provided.

As used herein, the term “biparatopic antibody” refers to, for example, an antibody that is capable of binding to two different epitopes on a single target (e.g., polypeptide). In one embodiment, one of the binding specificities of a biparatopic antibody as described herein is directed towards an epitope present on the first of the three immunoglobulin-like domains (IgI, IgII and IgIII) present in the extracellular domain of FGFR2 and the second binding specificity is directed to the second or third immunoglobulin-like domains. In another embodiment, a first binding specificity is directed to the second immunoglobulin-like domain of FGFR2 and the second binding specificity is directed to the third immunoglobulin-like domain. In another embodiment, a biparatopic antibody as described herein is directed towards an epitope present on the signal peptide (SP) and/or an epitope present in the second or third immunoglobulin-like domain of FGFR2 (i.e., Ig2 or Ig3) domains. In various embodiments, biparatopic antibodies as described herein are directed to combinations of such epitopes. For example, against an SP and IgI, Ig2, or Ig3, or against IgI and Ig2 or Ig3; or against Ig2 and Ig3.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “complementarity determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. In various aspects and embodiments, antibodies comprising modifications in these hybrid hypervariable positions are provided. The variable domains of native heavy and light chains each comprise four framework regions that primarily adopt a beta-sheet configuration, connected by three CDRs, which form loops that connect, and in some cases form part of, the .beta.-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

“Detect” refers to identifying the presence, absence or amount of the analyte to be detected. In some embodiments, the analyte is an antigen, epitope, or fragment thereof. In one embodiment, the term “detect” refers to detecting antibody binding to an agent of interest.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. In some embodiments, an antibody as described herein is directly or indirectly linked to a detectable label.

By “disease” is meant any condition or disorder that damages or interferes with the normal function of a cell, tissue, or organ. Examples of diseases include cancer (e.g., CCA, endometrial cancer, melanoma, esophageal cancer, bladder cancer, breast and lung cancer), as well as other hyperproliferative disorders that are associated with aberrant FGFR2 activity.

By “effective amount” is meant the amount of an agent required to ameliorate the symptoms of a disease relative to an untreated patient. The effective amount of active compound(s) used to practice methods for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, an effective amount of an agent is the amount of a biparatopic antibody required to block binding to FGFR2 or reduce FGFR2 activity.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

By “Fibroblast Growth Factor Receptor (FGFR)” is meant a receptor that binds to an FGF ligand, which binding typically induces tyrosine kinase activity. Exemplary FGFRs include FGFR1, FGFR2, FGFR2 alpha IIIb, FGFR3, and FGFR4.

The term “FGFR2” refers to fibroblast growth factor receptor 2 that is a member of the receptor tyrosine kinase superfamily. The nucleic acid and amino acid sequences of FGFR2 are known, and have been published in GenBank Accession Nos. NM_000141.4, NM_001144913.1, NM_001144914.1, NM 001144915.1, NM_001144916.1, NM_001144917.1, NM_001144918.1, NM_001144919.1, NM 022970.3, NM 023029.2.

An exemplary amino acid sequence of FGFR2 is provided at NP_000132, which is reproduced below (SEQ ID NO: 15).

| | |
|---|---|
| 1 | mvswgrficl vvvtmatlsl arpsfslved ttlepeeppt kyqisqpevy vaapgeslev |
| 61 | rcllkdaavi swtkdgvhlg pnnrtvlige ylqikgatpr dsglyactas rtvdsetwyf |
| 121 | mvnvtdaiss gddeddtdga edfvsensnn krapywtnte kmekrlhavp aantvkfrcp |
| 181 | aggnpmptmr wlkngkefkq ehriggykvr nqhwslimes vvpsdkgnyt cvveneygsi |
| 241 | nhtyhldvve rsphrpilqa glpanastvv ggdvefvckv ysdaqphiqw ikhvekngsk |
| 301 | ygpdglpylk vlkaagvntt dkeievlyir nvtfedagey tclagnsigi sfhsawltvl |
| 361 | papgrekeit aspdyleiai ycigvfliac mvvtvilcrm knttkkpdfs sqpavhkltk |
| 421 | riplrrqvtv saessssmns ntplvrittr lsstadtpml agvseyelpe dpkwefprdk |
| 481 | ltlgkplgeg cfgqvvmaea vgidkdkpke avtvavkmlk ddatekdlsd lvsememmkm |
| 541 | igkhkniinl lgactqdgpl yviveyaskg nlreylrarr ppgmeysydi nrvpeeqmtf |
| 601 | kdlvsctyql argmeylasq kcihrdlaar nvlvtennvm kiadfglard innidyykkt |
| 661 | tngrlpvkwm apealfdrvy thqsdvwsfg vlmweiftlg gspypgipve elfkllkegh |
| 721 | rmdkpanctn elymmmrdcw havpsqrptf kqlvedldri ltlttneeyl dlsqpleqys |
| 781 | psypdtrssc ssgddsvfsp dpmpyepclp qyphingsvk t |

Structurally, a FGFR2 amino acid sequence is a receptor tyrosine kinase protein having a signal peptide, at least one or more immunoglobulin (Ig)-like domains, an acidic box, a transmembrane domain, and a split tyrosine kinase domain and has over its full length at least about 90%, 91%, 92%, 93%, 94%, 95%, 96%, 97%, 98%, 99%, or 100% sequence identity with the amino acid sequence of GenBank accession numbers NM_000141.4, NM_001144913.1, NM_001144914.1, NM_001144915.1, NM_001144916.1, NM_001144917.1, NM_001144918.1, NM_001144919.1, NM 022970.3, NM_023029.2. Structurally, a FGFR2 nucleic acid sequence has over its full length at least about 90%, 91%, 92%, 93%, 94%, 95%, 96%, 97%, 98%, 99%, or 100% sequence identity with the nucleic acid sequence of GenBank accession numbers NM_000141.4, NM_001144913.1, NM_001144914.1, NM 001144915.1, NM_001144916.1, NM_001144917.1, NM_001144918.1, NM 001144919.1, NM_022970.3, NM_023029.2. The FGFR2 signal peptide may be retained or cleaved off.

By “FGFR2 activity” is meant tyrosine kinase activity.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, the term “fusion protein” or simply “fusion” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., 2012, Bioorg. Med. Chem., 20:571-582). Exemplary FGFR2 fusion proteins occur in cancers that have undergone genomic rearrangement. Such fusion proteins can be recombinantly expressed using methods and sequences that are known in the art and described herein.

As used herein, the term “human antibody” refers to an antibody in which substantially every part of the protein (e.g., CDR, framework, C_{L}, C_{H} domains (e.g., C_{H1}, C_{H2}, C_{H3}), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell (e.g., yeast) that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of some aspects and embodiments is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of some aspects and embodiments herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of some aspects and embodiments that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of some aspects and embodiments herein. An isolated polypeptide of some aspects and embodiments herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies (e.g., US2007/0178552, WO 96/027011, WO 98/050431 and Zhu et al. (1997) Protein Science 6:781-788). This is especially useful in driving the pairing of two different heavy chains together during the manufacture of biparatopic antibodies. For example, biparatopic antibodies having KnH in their Fc regions can further comprise single variable domains linked to each Fc region, or further comprise different heavy chain variable domains that pair with similar or different light chain domains. KnH technology can be also be used to pair two different receptor extracellular domains together or any other polypeptide sequences that comprises different target recognition sequences (e.g., including affibodies, peptibodies and other Fc fusions).

Biparatopic antibodies are also obtained using methods that do not depend on KnH technology. Labrijn, et al. (Controlled Fab-arm exchange for the generation of stable bispecific IgG1, Nature Protocols 9: 2450-2463, 2014) describe controlled Fab-arm exchange (cFAE), which is an easy-to-use method to generate bispecific IgG1 (bsIgG1) and biparatopic antibodies. The protocol involves the following: (i) separate expression of two parental IgG1s containing single matching point mutations in the CH3 domain; (ii) mixing of parental IgG1s under permissive redox conditions in vitro to enable recombination of half-molecules; (iii) removal of the reductant to allow reoxidation of interchain disulfide bonds; and (iv) analysis of exchange efficiency and final product using chromatography-based or mass spectrometry (MS)-based methods. The protocol generates bsAbs with regular IgG architecture, characteristics and quality attributes both at bench scale (micrograms to milligrams) and at a mini-bioreactor scale (milligrams to grams) that is designed to model large-scale manufacturing (kilograms). Starting from good-quality purified proteins, exchange efficiencies of ≥95% can routinely be obtained within 2-3 d (including quality control). In some embodiments, the two parental IgG1s contain matching point mutations, one in either IgG1, at the C_{H3}:C_{H3} interface, i.e., K409R and F405L, respectively (EU numbering conventions).

In one particular embodiment, Labrijn, et al. (Efficient generation of stable bispecific IgG1 by controlled Fab-arm exchange, PNAS. 110: 5145-5150, 2013) describe proof-of-concept studies with HER2×CD3 (T-cell recruitment) and HER2×HER2 (dual epitope targeting) bsAbs, which demonstrate superior in vivo activity compared with parental antibody pairs. Each of the aforementioned Labrijn publications is incorporated by reference herein in its entirety.

Methods useful for generating biparatopic antibodies are described, for example, in U.S. Pat. Nos. 9,212,230, 9,150,663, 10,344,050, each of which is incorporated herein by reference in its entirety.

As used herein, the term “operatively linked” in the context of a polynucleotide fragment is intended to mean that the two polynucleotide fragments are joined such that the amino acid sequences encoded by the two polynucleotide fragments remain in-frame.

By “reduces” is meant a negative alteration of at least 10%, 25%, 50%, 75%, or 100%.

By “reference” is meant a standard or control condition. In some embodiments, a neoplastic cell is contacted by an antibody described herein and the effect of the antibody binding to an antigen on the cell is determined relative to a corresponding reference cell not contacted with the antibody. In some embodiments, the reference is the proliferation, cell survival, or cell death observed in the control cell.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of a scFv molecule can be derived from one or more antibody molecules. It will also be understood by one of ordinary skill in the art that the variable regions of the scFv molecules of some aspects and embodiments herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

By “specifically binds” is meant a polypeptide or antibody that recognizes and binds a polypeptide of interest (e.g., an FGFR, such as FGFR2), but which does not substantially recognize and bind other molecules in a sample, for example, a biological sample, which naturally includes a polypeptide of some aspects and embodiments herein. An antibody or antigen-binding fragment thereof that specifically binds to an antigen will bind to the antigen with a K_{D} of less than 100 nM. For example, an antibody or antigen-binding fragment thereof that specifically binds to an antigen will bind to the antigen with a K_{D} of up to 100 nM (e.g., between 1 pM and 100 nM). An antibody or antigen-binding fragment thereof that does not exhibit specific binding to a particular antigen or epitope thereof will exhibit a K_{D} of greater than 100 nM (e.g., greater than 500 nm, 1 uM, 100 uM, 500 uM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

Nucleic acid molecules useful in the methods of some aspects and embodiments herein include any nucleic acid molecule that encodes a polypeptide of some aspects and embodiments herein or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of some aspects and embodiments herein include any nucleic acid molecule that encodes a polypeptide of some aspects and embodiments herein, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.10% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “transfecting” or “transfection” is used synonymously and according to some aspects and embodiments herein means the introduction of heterologous nucleic acid (DNA/RNA) into a eukaryotic cell, in particular yeast cells.

According to some aspects and embodiments herein, antibody fragments are understood as meaning functional parts of antibodies, such as Fc, Fab, Fab′, Fv, F(ab′)₂, scFv. According to some aspects and embodiments herein, corresponding biological active fragments are to be understood as meaning those parts of antibodies which are capable of binding to an antigen, such as Fab, Fab′, Fv, F(ab′)₂, and scFv.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein the term “variable region CDR” includes amino acids in a CDR or complementarity determining region as identified using sequence or structure based methods. As used herein, the term “CDR” or “complementarity determining region” refers to the noncontiguous antigen-binding sites found within the variable regions of both heavy and light chain polypeptides. These particular regions have been described by Kabat et al., J. Biol. Chem. 252:6609-6616, 1977 and Kabat, et al., Sequences of Proteins of Immunological Interest, Fifth Edition, U.S. Department of Health and Human Services, NIH Publication No. 91-3242, 1991; by Chothia et al., (J. Mol. Biol. 196:901-917, 1987), and by MacCallum et al., (J. Mol. Biol. 262:732-745, 1996) where the definitions include overlapping or subsets of amino acid residues when compared against each other. In certain embodiments, the term “CDR” is a CDR as defined by Kabat based on sequence comparisons.

As used herein, the term “vector” includes a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors of some aspects and embodiments herein contain a polynucleotide sequence as well as, e.g., additional sequence elements used for the expression of proteins and/or the integration of these polynucleotide sequences into the genome of a mammalian cell. Certain vectors that can be used for the expression of antibodies and antibody fragments of some aspects and embodiments herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors of some aspects and embodiments herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “VH” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A provides a schematic showing FGFR2 fusion proteins.

FIG. 1B provides three graphs quantifying population doubling in cells. FGFR2-fusions transformed BaF3 cells. BaF3 cells were transduced with retroviral vectors expressing fusions of the FGFR2 receptor tyrosine kinase domain with Phosphoglycerate Dehydrogenase (PHGDH), Adenosylhomocysteinase Like 1 AHCYL1, or BicC Family RNA Binding Protein 1 (BICC1) in the presence or absence of IL3. The FGFR2-AHCYL1 and FGFR2-PHGDH fusions FGFR2-BICC1(partially) conferred IL3-independent growth on BaF3 cells in an IL3 depletion assay.

FIG. 2 provides three graphs quantifying viability as a function of FGFR inhibitor NVP-BGJ398 dosage. NVP-BGJ398, also known as Infigratinib, is an FDA-approved, orally administrable, and selective FGFR inhibitor for FGFR1/2/3. NVP-BGJ398 is reported to have an IC50 of 0.9 nM/1.4 nM/1 nM in cell-free assays using FGFR1/2/3, respectively. The data demonstrate that FGFR2-fusion transformed BaF3 cells are sensitive to an FGFR inhibitor (NVP-BGJ398). A table showing IC50 (μM) is also provided.

FIG. 3A provides micrographs showing focus formation of NIH3T3 cells expressing FGFR2 fusion proteins.

FIG. 3B is a graph showing the number of colonies present in cultures of NIH3T3 cells expressing the FGFR2 fusions. This data demonstrates that the FGFR2-fusions were sufficient to transform NIH3T3 cells.

FIGS. 4A-4F present schematic diagrams, blots, images, tables and graphs showing that the FGFR2 extracellular domain is important for FGFR2 fusion driven cell growth and transformation and that patient-derived FGFR2 extracellular domain mutations increased transformation capacity. As also shown, some parental antibodies were effective in inhibiting ECD mutation driven cell growth. FIG. 4A is a graph showing that FGFR2-BICC1, FGFR2-AHCYL1, and FGFR2-PHGDH fusions grow faster compared to controls and respond to FGF ligands. The FGFR2 extracellular domain (ECD) contributes to the growth of transformed NIH3T3 cells expressing FGFR2 fusions. FIG. 4B shows diagrams of ECD deletion constructs of FGFR2-BICC1 fusions in which either the Ig1, Ig2, Ig3, or both the Ig2 and Ig3 extracellular domains of the FGFR2-BICC1 fusion was deleted. Such deletion mutants were used in cell-based assays to assess the importance of the ECDs in FGFR2-BICC1 fusions for causing oncogenic transformation in NIH3T3 and BaF3 cells. In addition, the ability of the antibodies described herein to inhibit the growth of the cells that overexpressed the ECD mutant FGFR2 fusions was assessed. FIG. 4C presents blots showing levels of expression of the ECD mutant FGFR2 fusions in transformed cells. FIG. 4D shows images of NIH3T3 cells transformed with each of the mutant ECD FGFR2-BICC1 fusions (FGFR2-BICC1 variants), and graphs showing the transformation capacities and growth effects of each of the FGFR2-BICC1 variants following transformation of the cells with the variants. Representative images in FIG. 4D show colonies formed upon transducing NIH3T3 cells with different FGFR2-BICC1 variants in a focus formation assay. The assay was performed with 5 replicates for each variant. FIG. 4E demonstrates that deletion mutations in the ECD of FGFR2 derived from patients having cholangiocarcinoma (e.g., Patients 1-4, (PT1-PT4)) increased transformation capacity when introduced into NIH3T3 cells and thus were activating mutations, as shown in the photographic images of transformed cell colonies and in the graphs depicting the number of transformed colonies formed. FIG. 4F illustrates that some parental antibodies (e.g., Antibodies A-F) were effective in inhibiting ECD mutation driven cell growth of cells that had been transformed with the patient-derived FGFR2 having ECD activating deletion mutations. In the graph in FIG. 4F, the results of assays using cells transformed with an FGFR2 having an ECD activating deletion mutation derived from Patient 1 (as presented in the table in FIG. 4F) and contacted with the denoted antibodies are shown in first set of boxes from the left; the results of assays using cells transformed with an FGFR2 having ECD activating deletion mutation derived from Patient 2 (as presented in the table in FIG. 4F) and contacted with the denoted antibodies are shown in the second set of boxes from the left; the results of assays using cells transformed with an FGFR2 having ECD activating deletion mutation derived from Patient 3 (as presented in the table in FIG. 4F) and contacted with the denoted antibodies are shown in the third set of boxes from the left; and the results of assays using cells transformed with an FGFR2 having ECD activating deletion mutation derived from Patient 4 (as presented in the table in FIG. 4F) and contacted with the denoted antibodies are shown in the rightmost set of boxes in the graph.

FIG. 5 is a graph showing that FGFR2-BICC1, FGFR2-AHCYL1, and FGFR2-PHGDH fusions are sensitive to NVP-BGJ398 treatments—indicating that these fusions signal via FGFR. NIH3T3 cells transformed with FGFR2 fusions are sensitive to the FGFR inhibitor NVP-BGJ398.

FIGS. 6A and 6B are graphs showing that FGF ligand (FGF10) augments the growth of FGFR2-PHGDH fusion expressing BaF3 cells.

FIG. 7A includes a schematic diagram and graphs. The schematic diagram indicates the FGFR2 epitopes bound by the indicated antibodies. Regions of the FGFR2 extracellular domain bound by antibody include the signal peptide (SP) and three immunoglobulin-like domains (IgI, IgII and IgIII). The FGF receptor also includes a transmembrane domain (TM) and two kinase domains. At the left of the figure, a series of histograms showing the results of FACS analyses using antibodies A-F are provided. At the right of the figure, the percent of cells that are GFP positive is shown as a function of the log of antibody concentration for antibodies A-F against the various FGFR2 domains shown in the schematic.

FIG. 7B is a table showing the sources, alternative nomenclature designations, and properties of antibodies A-F as described and used herein. By way of example, in FIG. 7B, alternative designations for “Antibody A” are “Bayer” and “M048-D01;” alternative designations for “Antibody B” are “Gal23” and “GAL-FR23;” alternative designations for “Antibody C” are “N10164,” “N10” and “10164;” alternative designations for “Antibody D” are “GE” and “2B 1.3.12;” alternative designations for “Antibody E” are “GA” and “HuGAL-FR21;” and alternative designations for “Antibody F” are “N12433,” “N12” and “12433”.

FIG. 8 is a schematic (described at FIG. 7A) and a graph showing the growth of BaF3 cells expressing FGFR2IIIb when treated with FGF10. Anti-FGFR2 antibodies blocked ligand-dependent stimulation of BaF3 cells expressing wild-type FGFR2b. FGFR2b is an isoform of FGFR2 that is predominantly expressed in cholangiocarcinoma.

FIG. 9 is a graph showing that Antibody F has inhibitory activity against the FGFR2-PHDGH activating fusion in BaF3 cells. Antibodies A and C and to a lesser extent D have agonist activity in the absence of FGF ligands.

FIG. 10 is a graph showing that antibodies C, D, E and F have activity against ligand stimulated growth of FGFR2-PHDGH fusion expressing BaF3 cells.

FIGS. 11A-11D illustrate the generation of biparatopic antibodies. FIG. 11A shows FGFR2 antibody combinations that are likely to be useful in generating biparatopic antibodies. The combinations of parental anti-FGFR2 antibodies increased the inhibitory effects of the antibodies on cell (e.g., BaF3) growth. FIGS. 11B and 11C provide designs for the generation of biparatopic antibodies. FIG. 11C provides a schematic of the generation of a biparatopic antibody based on duobody technology (Genmab). By way of example, duobody antibodies (e.g., IgG1 antibodies) are made by controlled Fab arm exchange of matched (destabilizing) mutations in the C_{H3} domains of the antibodies. K409R and F405L are examples of such destabilizing mutations in the C_{H3} interface. The complementary mutations favor heterodimerization. The generation of stable bispecific antibodies by controlled Fab arm exchange is reported by A. F. Labrijn et al., March 2013, Proc. Natl. Acad. Sci. USA, 110(13):5145-5150 and A. F. Labrijn et al., May 2017 (online), Nature Scientific Reports, 7:2476. FIG. 11D illustrates the purification of bispecific antibodies using nickel purification to separate and purify homodimeric antibodies (untagged or His/His tagged antibodies) from heterodimeric biparatopic, His-tagged antibodies, which comprise a His tag in only one-half of the antibody (bispecific).

FIG. 12 includes two graphs. The left graph shows the binding shift analyzed by FACS using a SNU-16 cell line with FGFR2 amplification. The right graph shows the percent of cells bound to FGFR2 antibodies (with GFP+) at various concentrations of GA antibodies. In previous studies the GA antibody had an approximately 1 nM Kd (nM) (FACS). In the present study, Antibody E had 1.58 nM Kd. Kd is calculated based on the binding curve. The GA antibody (HuGAL-FR21 from Galaxy) is described in US Patent Publication No. 20160362496. In the graph on the right, the percent number of cells with positive fluorescence is shown as a function of the log of Galaxy antibody concentration.

FIG. 13 provides FACS data (at the top) and % binding (at the bottom), which were used to calculate Kd. Each FACS curve represents differing concentrations. The further the shift is to the right—the higher the Ab concentration.

FIGS. 14A-14E show charts, isobolograms and Loewe scores demonstrating that FGFR2 bivalent antibodies synergize with FGFR inhibitors to inhibit FGFR2 fusion driven cell growth in the absence of FGF ligand (FGF10). FIG. 14A presents charts showing synergy between the FGFR2 small molecule inhibitor BGJ398 (also termed NVP-BGJ398), at concentrations of 0, 0.84, 1.69, 3.39, 6.78 μM, (see, e.g., Example 3), and an anti-FGFR2 bivalent antibody (Antibody F) at concentrations of 0, 5, 15, 30, 40 μg/mL in the absence of FGF ligand (−FGF) in a cell-based assay using BaF3 cells overexpressing an FGFR2 fusion molecule (FGFR2-PHGDH) after 3, 4 and 5 days of treatment with the inhibitor and the antibody. In the charts in FIG. 14A, the lighter color squares and numerical values correlate with less cell survival, demonstrating that in the absence of FGF, Antibody F shows an inhibitory effect with BGJ398. FIGS. 14B-14D provide isobolograms showing synergy between FGFR inhibitor and the anti-FGF2 bivalent antibody on the different days and supporting the results shown in FIG. 14A. FIG. 14B corresponds to 3 days after treatment with the BGJ398 inhibitor; FIG. 14C corresponds to 4 days after treatment with the BGJ398 inhibitor; and FIG. 14D corresponds to 5 days after treatment with the BGJ398 inhibitor. FIG. 14E provide Loewe scores showing synergy between the FGFR inhibitor and the anti-FGF2 bivalent antibody on the different days and supporting the results shown in FIG. 14A. The results show that in the absence of FGF10, BGJ398 synergizes with antibody F to inhibit FGFR2-PHGDH driven BaF3 cell growth when treated for 5 days.

FIGS. 15A-15E show charts, isobolograms and Loewe scores demonstrating that FGFR2 bivalent antibodies synergize with FGFR inhibitors to inhibit FGFR2 fusion driven cell growth in the presence of FGF ligand (FGF10). FIG. 15A presents charts showing synergy between the FGFR2 small molecule inhibitor BGJ398 and an anti-FGFR2 bivalent antibody (Antibody D) in the presence of FGF ligand (+FGF) in a cell-based assay using BaF3 cells overexpressing an FGFR2 fusion molecule (FGFR2-PHGDH) after 3, 4 and 5 days of treatment with the inhibitor and the antibody. In the charts in FIG. 15A, the lighter color squares and numerical values correlate with less cell survival, demonstrating that in the presence of FGF, Antibody D shows an inhibitory effect with BGJ398. FIGS. 15B-15D provide isobolograms showing synergy between the FGFR inhibitor and the anti-FGF2 bivalent antibody on the different days and supporting the results shown in FIG. 15A. FIG. 15B corresponds to 3 days after treatment with the BGJ398 inhibitor; FIG. 15C corresponds to 4 days after treatment with the BGJ398 inhibitor; and FIG. 15D corresponds to 5 days after treatment with the BGJ398 inhibitor. FIG. 15E provide Loewe scores showing synergy between the FGFR inhibitor and the anti-FGF2 bivalent antibody on the different days and supporting the results shown in FIG. 15A. In the presence of FGF10, BGJ398 synergizes with antibody D to inhibit FGFR2-PHGDH driven BaF3 cells growth when treated for 5 days. In an embodiment, another FGFR2 fusion, e.g., FGFR2-BICC1, may be used with NIH3T3 cells, e.g., NIH3T3 cells overexpressing FGFR2-BICC1, to assess the synergy between BGJ398 and Antibodies F and D, with and without FGF ligand.

FIG. 16A-16F provide schematic illustrations, blots and graphs related to the development of a NANOBIT® assay to measure FGFR2 dimerization. Such an assay is used to screen biparatopic screening for their ability to disrupt FGFR2 dimerization. FIG. 16A shows that FGFR2 receptor dimerization is measured using the NANOBIT® assay as depicted by receptor dimerization resulting from FGF ligand binding to the ECD of FGFR2 (left) and by receptor dimerization resulting from FGFR2 fusions (right). The protein interactions bring the subunits into close proximity to form a functional enzyme that generates a bright, luminescent signal. (FIG. 16B). FIG. 16C (left) shows Western blots of NANOBIT® expression constructs of FGFR2-WT (wild type), FGFR2-ACHYL1 and FGFR2-BICC1 used for transient expression in HEK293T cells at 3 days post transfection. FIG. 16C (right) shows a graph of the results of the NANOBIT® assay using FGFR2 fusions and FGFR2 expressing cells. FIG. 16D shows blots of NANOBIT® constructs of FGFR2-WT (wild type), FGFR2-ACHYL1 and FGFR2-BICC1 used for stable expression of the FGFR2 fusions in HEK293T cells. FIG. 16E presents a graph of the results of the NANOBIT® assay using NANOBIT® constructs with or without FGF10 ligand. FIG. 16F shows the results of assays in which the FGFR2 expressing stable cells lines were subjected to Antibodies A-F (as identified in FIG. 7B) and the NANOBIT® assay was performed. As shown in the graph on the left side of FIG. 16F, certain of the antibodies A-F inhibited the growth of BaF3 cells stably overexpressing FGFR2 (FGFR2IIIb). Antibody D was used in the NANOBIT® assay (graph on the right of FIG. 16F). In the rightmost graph in FIG. 16F, the fold luminescence resulting from FGFR2 WT (wild type) in the assay is represented by the left bar in each set of three bars; the fold luminescence resulting from FGFR2+FGF10 ligand in the assay is represented by the middle bar in each set of three bars; and the fold luminescence resulting from +/−FGF10 ligand difference in the assay is represented by the right bar in each set of three bars shown in FIG. 16F.

FIGS. 17A-17C provide a schematic and tabular data related to assessments made to compare the avidity of biparatopic antibodies to that of their parental (bivalent monospecific) antibodies. FIG. 17A illustrates that biparatopic antibodies, which are bivalent and bispecific, were found to bind more tightly to ligand (FGFR2) than their parental (bivalent monospecific) antibodies and, as such, the biparatopic antibodies that bind more tightly to FGFR2 are likely to be more efficient in blocking the FGFR2 dimerization. FIGS. 17B and 17C present tables showing the binding affinities among parental and biparatopic antibodies as measured using Surface Plasmon Resonance (SPR). In the tables, nearly all of the parental monospecific antibodies, namely, FGFR_B (KD (M) 1.6E-08), FGFR_N_12433 (KD (M) 7.5E-09), FGFR_GA (KD (M) 7.5E-09), FGFR_Gal23 (KD (M) 2.1E-09), FGFR_N_10164 (KD (M) 1.7E-09), and FGFR_GE_FL (KD (M) 9.9E-10), showed lower binding affinities than the biparatopic antibodies that were assessed in the binding assays. The nomenclature of the biparatopic antibodies is based on the descriptions of the antibodies shown in the table in FIG. 7B.

FIGS. 18A-18D present graphs showing the ability of biparatopic antibodies to impact FGFR2-fusion driven cell growth in BaF3 cells molecularly engineered to overexpress FGFR2-PHGDH fusion. As shown in FIGS. 18A-18D, biparatopic antibodies GA/N12, GA/Gal23, Gal23/N12, and B/N12 were more efficient and potent at inhibiting the growth of BaF3 cells overexpressing FGFR2-PHGDH fusion (found in cholangiocarcinoma patients) compared to control cells overexpressing empty vector. These biparatopic antibodies were also more efficient at inhibiting growth of BaF3 cells overexpressing the FGFR2-PHGDH fusion compared to their parental antibodies. The nomenclature of the biparatopic antibodies is based on the designations and descriptions of the parental antibodies presented in FIG. 7B.

FIGS. 19A-19E present graphs showing the ability of biparatopic antibodies to impact FGFR2-fusion driven cell growth in NIH3T3 cells molecularly engineered to overexpress FGFR2-BICC1 fusion. As shown in FIGS. 19A-19E biparatopic antibodies GE/N10, GE/N12, B/GE, B/GA, and B/N12 were more efficient and potent at inhibiting growth of NIH3T3 cells overexpressing the FGFR2-BICC1 fusion (found in cholangiocarcinoma patients) compared to their parental antibodies. The nomenclature of the biparatopic antibodies is based on the designations and descriptions of the parental antibodies presented in FIG. 7B.

### DETAILED DESCRIPTION OF THE EMBODIMENTS

Featured herein are antagonistic biparatopic antibodies that specifically bind and inhibit an FGF receptor (e.g., FGFR2) and methods of using such antibodies for the treatment of cancers, including Cholangiocarcinoma (CCAs).

The aspects and embodiments described herein are based, at least in part, on the discovery of biparatopic antibodies that bind two different epitopes on the fibroblast growth factor receptor 2 (FGFR2) and that inhibit FGFR2 activity. Without wishing to be bound by theory, biparatopic antibodies of some aspects and embodiments herein inhibit FGFR2 not only by blocking ligand binding to FGFR2, but also by sterically blocking interaction/dimerization between FGF receptors.

Six antibodies that bind to distinct epitopes in the extracellular domain of FGFR2 are used to generate biparatopic antibodies against FGFR2. The V_{H} and V_{L} sequences of those antibodies are provided below:

M048-D01 VH
(SEQ ID NO: 1)
EVQLLESGGGLVQPGGSLRLSCAASGFTFSSYAMSWVRQAPGKGLEWVSA
ISGSGTSTYYADSVKGRFTISRDNSKNTLYLQMNSLRAEDTAVYYCARVR
YNWNHGDWFDPWGQGTLVTVSS
M048-D01 VL
(SEQ ID NO: 16)
QSVLTQPPSASGTPGQRVTISCSGSSSNIGNNYVSWYQQLPGTAPKLLIY
ENYNRPAGVPDRFSGSKSGTSASLAISGLRSEDEADYYCSSWDDSLNYWV
FGGGTKLTVLG
HuGAL-FR21 VH
(SEQ ID NO: 17)
QVQLVQSGAEVKKPGSSVKVSCKASGYIFTTYNVHWVRQAPGQGLEWIGS
IYPDNGDTSYNQNFKGRATITADKSTSTAYMELSSLRSEDTAVYYCARGD
FAYWGQGTLVTVSS
HuGAL-FR21 VL
(SEQ ID NO: 18)
DIQMTQSPSSLSASVGDRVTITCKASQGVSNDYAWYQQKPGKAPKLLIYS
ASYRYTGVPSRFSGSGSGTDFTFTISSLQPEDIATYYCQQHSTTPYTFGQ
GTKLEIK
GAL-FR23 VH
(SEQ ID NO: 19)
QIQLVQSGPELKKPGETVKISCKASGYTFTDFGMNWMKQAPGKGFKWMGW
INTSTGESTYADDFKGRFAFSLETSASTAYLQINNLKNEDMATYFCARNS
YYGGSYGYWGQGTTLTVSS
GAL-FR23 VL
(SEQ ID NO: 20)
DIVMSQSPSSLAVSVGEKVTMKCKSSQSLLYSSNQKNYLAWYQQKPGQSP
KLLIYWASTRESGVPDRFTGSGSGTDFTLTISSVKAEDLAVYYCQQYYSY
PWTFGGGTKLEIK
2B 1.3.12 VH
(SEQ ID NO: 21)
EVQLVESGGGLVQPGGSLRLSCAASGFPFTSTGISWVRQAPGKGLEWVGR
THLGDGSTNYADSVKGRFTISADTSKNTAYLQMNSLRAEDTAVYYCARTY
GIYDTYDMYTEYVMDYWGQGTLVTVSS
2B 1.3.12 VL
(SEQ ID NO: 22)
DIQMTQSPSSLSASVGDRVTITCRASQDVDTSLAWYKQKPGKAPKLLIYS
ASFLYSGVPSRFSGSGSGTDFTLTISSLQPEDFATYYCQQSTGHPQTFGQ
GTKVEIK
10164 VH
(SEQ ID NO: 23)
QVQLVESGGGLVKPGGSLRLSCAASGFTFSSYALSWVRQAPGKGLEWVGR
IRSKIDGGTTDYAAPVKGRFTISRDDSKNTLYLQMNSLKTEDTAVYYCAR
DRSPSDSSAFAIWGQGTLVTVSS
10164 VL
(SEQ ID NO: 24)
DIELTQPPSVSVSPGQTASITCSGDNLGSQYVDWYQQKPGQAPVLVIYDD
NDRPSGIPERFSGSNSGNTATLTISGTQAEDEADYYCQSWDSLSVVFGGG
TKLTVLG
12433 VH
(SEQ ID NO: 25)
QVQLVQSGAEVKKPGESLKISCKGSGYSFTNYYIHWVRQMPGKGLEWMGA
IYPDNSDTTYSPSFQGQVTISADKSISTAYLQWSSLKASDTAMYYCARGA
DIWGQGTLVTVSS
12433 VL
(SEQ ID NO: 26)
DIQMTQSPSSLSASVGDRVTITCRASQDIDPYLSNWYQQKPGKAPKLLIY
DASNLQSGVPSRFSGSGSGTDFTLTISSLQPEDFATYYCQQTTSHPYTFG
QGTKVEIK

All possible (i.e., twenty-one) combinations of the six antibodies are used to generate biparatopic antibodies. In particular embodiments, the antibodies include specific mutations that create the light and heavy chain pairings. In other embodiments, DuoBody technology is used to create a biparatopic antibody. These biparatopic antibodies are tested in binding, proliferation, and dimerization assays to identify those antibodies binding to epitopes that allow simultaneous binding that has functional consequences.

### Cholangiocarcinoma

Cholangiocarcinoma (CCA) is the most common primary biliary tract malignancy and the second most common primary hepatic malignancy. Overall, CCAs account for 3% of all gastrointestinal malignancies. The incidence of CCA has increased over the past three decades; however, the 5-year survival remains at approximately 10%. Based on their anatomic location within the biliary tree, CCAs are classified into intrahepatic CCA (iCCA), perihilar CCA (pCCA), and distal CCA (dCCA) subtypes. Deregulation of growth factor tyrosine kinases, including the FGFR, EGFR, and EGFR pathways, plays a critical role in CCA initiation and progression. HGF, a ligand for the MET receptor, promotes tumor invasiveness and metastasis. Aberrant overexpression of HGF and MET occurs in CCA and is associated with a poor prognosis.

### Fibroblast Growth Factor (FGF) and the FGF Receptor (FGFR)

The FGF pathway includes 22 human FGFs and a number of transmembrane receptor tyrosine kinases, FGFR 1-4. FGF signaling is involved in a large number of biological processes including proliferation, differentiation, survival, migration, and angiogenesis. The FGF-FGFR axis is activated with binding of FGF to FGFR and heparin sulfate proteoglycan in a specific complex on the surface of the cell. In this complex, two molecules of heparin sulfate link two FGFs into a dimer that bridges two FGFR chains (2 FGF, 2 heparin, 2 FGFR). FGFR dimerization is homo-dimer driven. Once formed, this complex activates the FGFR tyrosine kinase, resulting in autophosphorylation. FGFR tyrosine kinase activity activates intracellular signaling cascades that promote cell survival and proliferation. Ras-MAPK, phosphatidylinositol 3-kinase (PI3K)-protein kinase Akt/protein kinase B pathways, and Src all play a role in this intracellular signaling cascade.

Aberrant FGFR signaling often results in oncogenic changes. Genomic alteration of FGFR can result from activating mutations, receptor gene amplifications, and chromosomal translocations. Intragenic translocations can lead to formation of a fusion protein consisting of a transcription factor fused to an FGFR kinase domain with consequent FGFR dimerization and activation. Genomic aberrations lead to ligand-independent FGF signaling. FGFR2 fusions, e.g., without limitation, FGFR2-PHGDH, FGFR2-AHCYL1 and FGFR2-BICC1, as described herein, have been observed in 10% to 16% of patients with intrahepatic CCA and can play a role in cell transformation, aberrant cell growth and oncogenesis.

Provided herein are biparatopic antibodies that specifically bind epitopes in the extracellular domain of FGFR2, and inhibit FGFR2 and FGFR2 fusion activity.

### Generation and Screening of Biparatopic Antibodies

Biparatopic antibodies that specifically bind FGFR2 are provided and described herein. In one example, a biparatopic antibody binds FGFR2 and inhibits ligand-driven receptor dimerization and tyrosine kinase activity. In another embodiment, a biparatopic antibody binds an FGFR2 fusion and inhibits ligand independent tyrosine kinase activity. In another embodiment, a biparatopic antibody binds FGFR2 and accelerates receptor internalization, thereby downregulating receptor function. Methods for generating antibodies against a protein of interest are known in the art.

When animals are immunized with antigens they respond by generating a polyclonal antibody response comprised of many individual monoclonal antibody specificities. It is the sum of these individual specificities that make polyclonal antibodies useful in so many different assays. Individual monoclonal antibodies were originally isolated by immortalizing individual B cells using hybridoma technology (Kohler and Milstein, Nature 256, 495, 2011), in which B cells from an immunized animal are fused with a myeloma cell. With the advent of molecular biology, in vitro methods to generate antibodies, including biparatopic antibodies, against proteins of interest have been developed.

The terms “antigen of interest” or “target protein” are used herein interchangeably and refer generally to the agent recognized and specifically bound by an antibody.

An antibody is a polypeptide chain-containing molecular structure with a specific shape that specifically binds an epitope, where one or more non-covalent binding interactions stabilize the complex between the molecular structure and the epitope. In one embodiment, an antibody molecule is an immunoglobulin (e.g., IgG, IgM, IgA, IgE, IgD). Antibodies from a variety of sources, e.g. human, rodent, rabbit, cow, sheep, pig, dog, or fowl are considered “antibodies.” Numerous antibody coding sequences have been described; and others may be raised by methods well-known in the art.

For example, antibodies, including biparatopic antibodies, or antigen binding fragments may be produced by genetic engineering. Antibody coding sequences of interest include those encoded by native sequences, as well as nucleic acids that, by virtue of the degeneracy of the genetic code, are not identical in sequence to a wild-type nucleic acid sequence. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues). Variants also include fragments of the polypeptides disclosed herein, particularly biologically active fragments and/or fragments corresponding to functional domains. Techniques for in vitro mutagenesis of cloned genes are known. Also included in some aspects and embodiments herein are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the variable light and heavy chain regions obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated fully herein by reference).

Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although apparently complex, the process is straightforward in practice. See, e.g., U.S. Pat. No. 6,187,287, incorporated fully herein by reference.

In addition to entire immunoglobulins (or their recombinant counterparts), immunoglobulin fragments comprising the epitope binding site (e.g., Fab′, F(ab′)₂, or other fragments) may be synthesized. “Fragment,” or minimal immunoglobulins may be designed utilizing recombinant immunoglobulin techniques. For instance “Fv” immunoglobulins for use in some aspects and embodiments herein may be produced by synthesizing a variable light chain region and a variable heavy chain region. Combinations of antibodies are also of interest, e.g. diabodies, which comprise two distinct Fv specificities.

Immunoglobulins may be modified post-translationally, e.g. to add chemical linkers, detectable moieties, such as fluorescent dyes, enzymes, substrates, chemiluminescent moieties and the like, or specific binding moieties, such as streptavidin, avidin, or biotin, and the like may be utilized in the methods and compositions of some aspects and embodiments herein.

Mapping Epitopes of FGFR2 that Promote Receptor Antagonism

Antagonistic FGFR2 biparatopic antibodies, and antigen-binding fragments thereof can be produced by screening libraries of polypeptides (e.g., antibodies and antigen-binding fragments thereof) for functional molecules that are capable of binding epitopes within FGFR2 that selectively promote receptor antagonism rather than receptor activation. Such epitopes can be modeled by screening antibodies or antigen-binding fragments thereof against a series of linear or cyclic peptides containing residues that correspond to a desired epitope within FGFR2.

As an example, peptides containing individual fragments isolated from FGFR2 that promote receptor antagonism can be synthesized by peptide synthesis techniques described herein or known in the art. These peptides can be immobilized on a solid surface and screened for molecules that bind antagonistic FGFR2 antibodies (e.g., biparatopic antibodies, and antigen-binding fragments thereof), such as antibodies A-F, e.g., using an ELISA-based screening platform using established procedures. Using this assay, peptides that specifically bind antibodies A-F with high affinity therefore contain residues within epitopes of FGFR2 that preferentially bind these antibodies. Peptides identified in this manner (e.g., peptide fragments of an FGFR2 extracellular domain, including for example immunoglobulin-like domains (IgI, IgII and IgIII) can be used to screen libraries of antibodies and antigen-binding fragments thereof in order to identify anti-FGFR2 antibodies useful in generating biparatopic antibodies of some aspects and embodiments herein. Moreover, since these peptides act as surrogates for epitopes within FGFR2 that promote receptor antagonism, antibodies generated using this screening technique are likely to bind the corresponding epitopes in FGFR2 and are expected to be antagonistic of receptor activity.

### Screening of Libraries for Antagonistic FGFR2 Polypeptides

Methods for high throughput screening of polypeptide (e.g., biparatopic antibody, or antibody fragment) libraries for molecules capable of binding epitopes within FGFR2 include, without limitation, display techniques including phage display, bacterial display, yeast display, mammalian display, ribosome display, mRNA display, and cDNA display. The use of phage display to isolate ligands that bind biologically relevant molecules has been reviewed, e.g., in Felici et al. (Biotechnol. Annual Rev. 1:149-183, 1995), Katz (Annual Rev. Biophys. Biomol. Struct. 26:27-45, 1997), and Hoogenboom et al. (Immunotechnology 4:1-20, 1998). Several randomized combinatorial peptide libraries have been constructed to select for polypeptides that bind different targets, e.g., cell surface receptors or DNA (reviewed by Kay (Perspect. Drug Discovery Des. 2, 251-268, 1995), Kay et al., (Mol. Divers. 1:139-140, 1996)). Proteins and multimeric proteins have been successfully phage-displayed as functional molecules (see EP 0349578A, EP 4527839A, EP 0589877A; Chiswell and McCafferty (Trends Biotechnol. 10, 80-84 1992)). In addition, functional antibody fragments (e.g. Fab, single-chain Fv [scFv]) have been expressed (McCafferty et al. (Nature 348: 552-554, 1990), Barbas et al. (Proc. Natl. Acad Sci. USA 88:7978-7982, 1991), Clackson et al. (Nature 352:624-628, 1991)). These references are hereby incorporated by reference in their entirety.

In addition to generating anti-FGFR2 polypeptides (e.g., biparatopic antibodies, and antigen-binding fragments thereof) of some aspects and embodiments herein, in vitro display techniques (e.g., those described herein and those known in the art) also provide methods for improving the affinity of an anti-FGFR2 polypeptide of some aspects and embodiments herein. For instance, rather than screening libraries of antibodies and fragments thereof containing completely randomized hypervariable regions, one can screen narrower libraries of antibodies and antigen-binding fragments thereof that feature targeted mutations at specific sites within hypervariable regions. This can be accomplished, e.g., by assembling libraries of polynucleotides encoding antibodies or antigen-binding fragments thereof that encode random mutations only at particular sites within hypervariable regions. These polynucleotides can then be expressed in, e.g., filamentous phage, bacterial cells, yeast cells, mammalian cells, or in vitro using, e.g., ribosome display, mRNA display, or cDNA display techniques in order to screen for antibodies or antigen-binding fragments thereof that specifically bind FGFR2 epitopes with improved binding affinity. Yeast display, for instance, is well-suited for affinity maturation, and has been used previously to improve the affinity of a single-chain antibody to a KD of 48 fM (Boder et al. (Proc Nat Acad Sci USA 97:10701, 2000)).

Additional in vitro techniques that can be used for the generation and affinity maturation of antagonistic FGFR2 polypeptides (e.g., single-chain polypeptides, antibodies, and antigen-binding fragments thereof) of some aspects and embodiments herein include the screening of combinatorial libraries of antibodies or antigen-binding fragments thereof for functional molecules capable of specifically binding FGFR2-derived peptides. Combinatorial antibody libraries can be obtained, e.g., by expression of polynucleotides encoding randomized hypervariable regions of an antibody or antigen-binding fragment thereof in a eukaryotic or prokaryotic cell. This can be achieved, e.g., using gene expression techniques described herein or known in the art. Heterogeneous mixtures of antibodies can be purified, e.g., by Protein A or Protein G selection, sizing column chromatography), centrifugation, differential solubility, and/or by any other standard technique for the purification of proteins. Libraries of combinatorial libraries thus obtained can be screened, e.g., by incubating a heterogeneous mixture of these antibodies with a peptide derived from FGFR2 that has been immobilized to a surface for a period of time sufficient to allow antibody-antigen binding. Non-binding antibodies or fragments thereof can be removed by washing the surface with an appropriate buffer (e.g., a solution buffered at physiological pH (approximately 7.4) and containing physiological salt concentrations and ionic strength, and optionally containing a detergent, such as TWEEN-20). Antibodies that remain bound can subsequently be detected, e.g., using an ELISA-based detection protocol (see, e.g., U.S. Pat. No. 4,661,445; incorporated herein by reference).

Additional techniques for screening combinatorial libraries of polypeptides (e.g., antibodies, and antigen-binding fragments thereof) for those that specifically bind FGFR2-derived peptides include the screening of one-bead-one-compound libraries of antibody fragments. Antibody fragments can be chemically synthesized on a solid bead (e.g., using established split-and-pool solid phase peptide synthesis protocols) composed of a hydrophilic, water-swellable material such that each bead displays a single antibody fragment. Heterogeneous bead mixtures can then be incubated with a FGFR2-derived peptide that is optionally labeled with a detectable moiety (e.g., a fluorescent dye) or that is conjugated to an epitope tag (e.g., biotin, avidin, FLAG tag, HA tag) that can later be detected by treatment with a complementary tag (e.g., avidin, biotin, anti-FLAG antibody, anti-HA antibody, respectively). Beads containing antibody fragments that specifically bind a FGFR2-derived peptide can be identified by analyzing the fluorescent properties of the beads following incubation with a fluorescently-labeled antigen or complementary tag (e.g., by confocal fluorescent microscopy or by fluorescence-activated bead sorting; see, e.g., Muller et al. (J. Biol. Chem., 16500-16505, 1996); incorporated herein by reference). Beads containing antibody fragments that specifically bind FGFR2-derived peptides can thus be separated from those that do not contain high-affinity antibody fragments. The sequence of an antibody fragment that specifically binds a FGFR2-derived peptide can be determined by techniques known in the art, including, e.g., Edman degradation, tandem mass spectrometry, matrix-assisted laser-desorption time-of-flight mass spectrometry (MALDI-TOF MS), nuclear magnetic resonance (NMR), and 2D gel electrophoresis, among others (see, e.g., WO 2004/062553; incorporated herein by reference).

### Methods of Identifying Antibodies and Ligands

Methods for high throughput screening of antibody, antibody fragment, and ligand libraries for molecules capable of binding FGFR2 can be used to identify antibodies suitable for use in a biparatopic antibody useful for treating CCA as described herein. Such methods include in vitro display techniques known in the art, such as phage display, bacterial display, yeast display, mammalian cell display, ribosome display, mRNA display, and cDNA display, among others. The use of phage display to isolate ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). These techniques, among others, can be used to identify and improve the affinity of antibodies that bind FGFR2.

### Host Cells for Expression of Antagonistic FGFR2 Antibodies

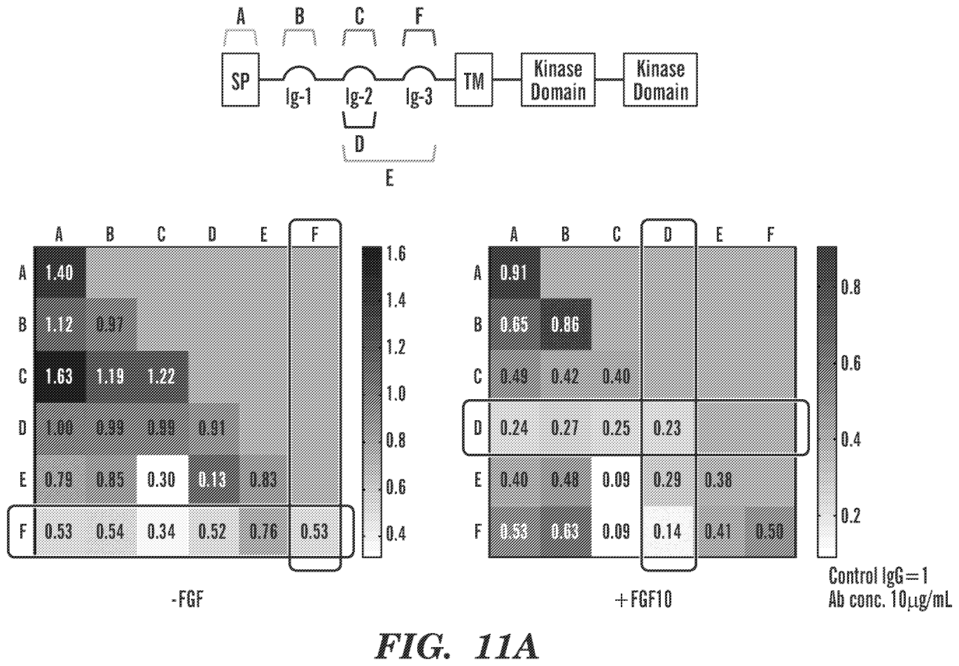

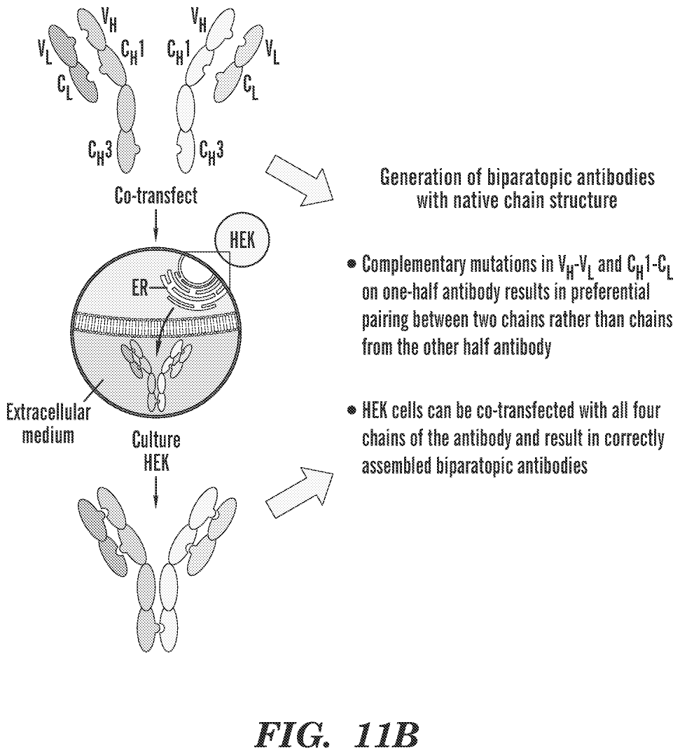

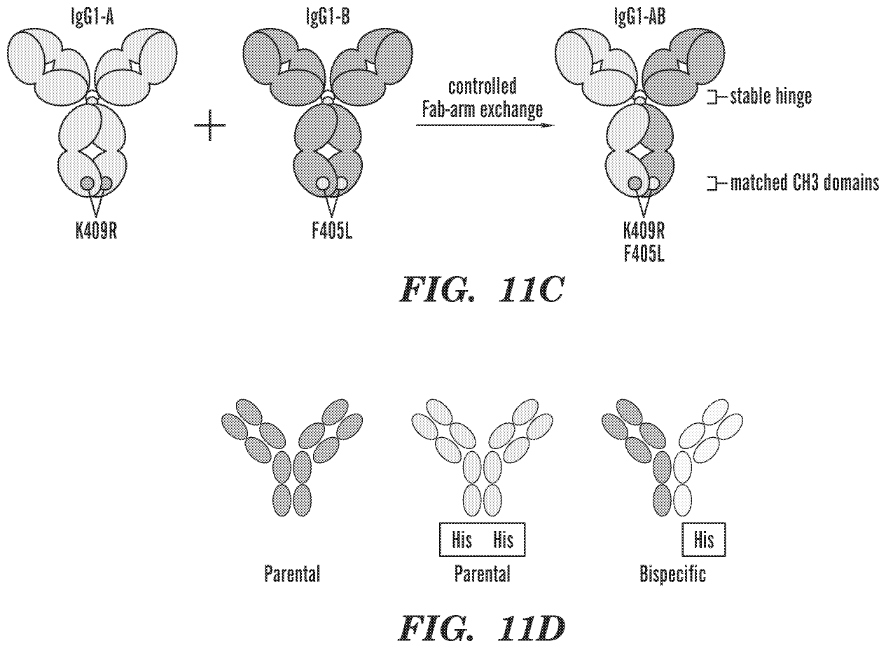

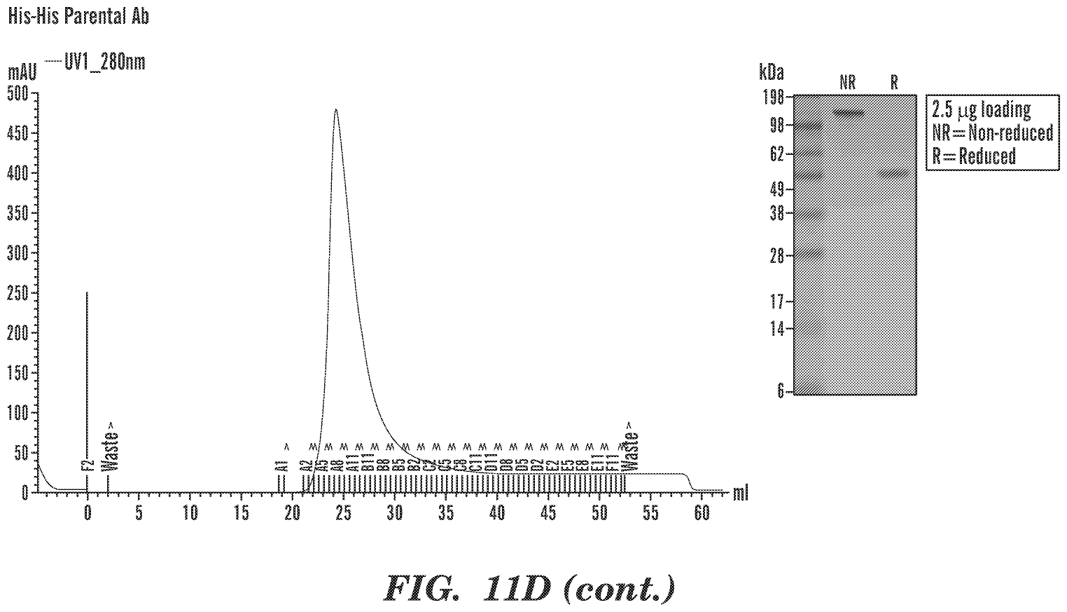

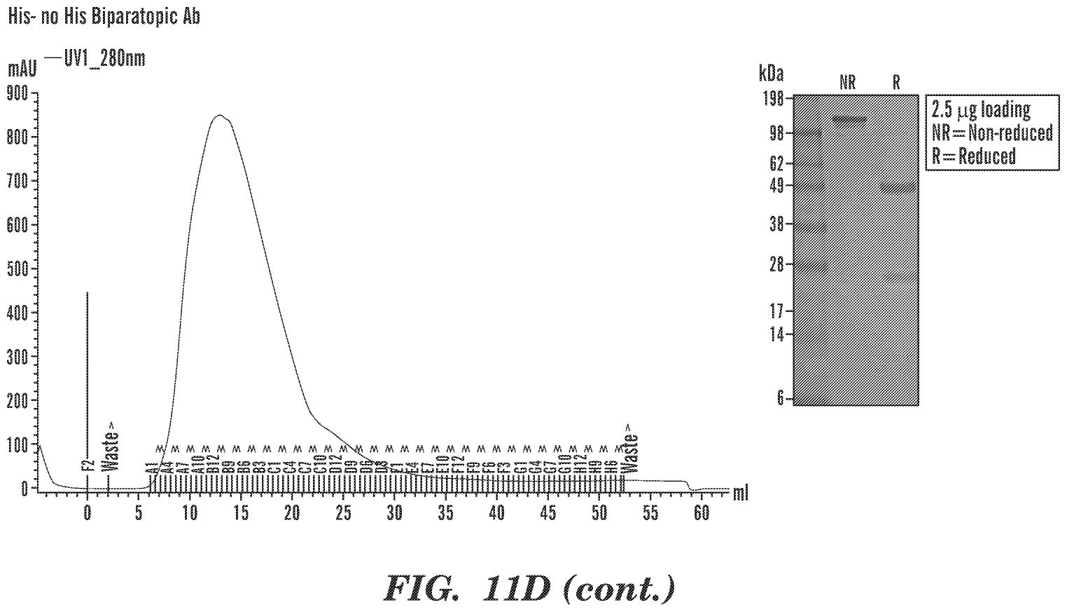

Mammalian cells can be co-transfected with polynucleotides encoding the antibodies of some aspects and embodiments herein, which are expressed as recombinant polypeptides, and assembled into biparatopic antibodies by the host cell. In one embodiment, a mammalian cell is co-transfected with polynucleotides encoding four chains of a biparatopic antibody, which expression results in the correct assembly of a biparatopic antibody (FIG. 11B).

It is possible to express antibodies (e.g., biparatopic antibodies, or antigen-binding fragments thereof) in either prokaryotic or eukaryotic host cells. In certain embodiments, expression of polypeptides (e.g., biparatopic antibodies, or antigen-binding fragments thereof) is performed in eukaryotic cells, e.g., mammalian host cells, for optimal secretion of a properly folded and immunologically active antibody. Exemplary mammalian host cells for expressing the recombinant antibodies or antigen-binding fragments thereof of some aspects and embodiments herein include Chinese Hamster Ovary (CHO cells) (including DHFR CHO cells, described in Urlaub and Chasin (1980, Proc. Natl. Acad. Sci. USA 77:4216-4220), used with a DHFR selectable marker, e.g., as described in Kaufman and Sharp (1982, Mol. Biol. 159:601-621), NSO myeloma cells, COS cells, HEK293T cells, SP2/0, NIH3T3, and BaF3 cells. Additional cell types that may be useful for the expression of antibodies and fragments thereof include bacterial cells, such as BL-21(DE3) E. coli cells, which can be transformed with vectors containing foreign DNA according to established protocols. Additional eukaryotic cells that may be useful for expression of antibodies include yeast cells, such as auxotrophic strains of S. cerevisiae, which can be transformed and selectively grown in incomplete media according to established procedures known in the art. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown.

Polypeptides (e.g., biparatopic antibodies, or antigen-binding fragments thereof) can be recovered from the culture medium using standard protein purification methods. Host cells can also be used to produce portions of intact antibodies, such as Fab fragments or scFv molecules. Also included in some aspects and embodiments herein are methods in which the above procedure is varied according to established protocols known in the art. For example, it can be desirable to transfect a host cell with DNA encoding either the light chain or the heavy chain (but not both) of an anti-FGFR2 antibody of some aspects and embodiments herein in order to produce an antigen-binding fragment of the antibody.

Once an anti-FGFR2 polypeptide (e.g., biparatopic antibodies, or antigen-binding fragments thereof) of some aspects and embodiments herein has been produced by recombinant expression, it can be purified by any method known in the art, such as a method useful for purification of an immunoglobulin molecule, for example, by chromatography (e.g., ion exchange, affinity, particularly by affinity for FGFR2 after Protein A or Protein G selection, and sizing column chromatography), centrifugation, differential solubility, or by any other standard technique for the purification of proteins. Further, the anti-FGFR2 polypeptides of some aspects and embodiments described herein, or antigen-binding fragments thereof, can be fused to heterologous polypeptide sequences described herein or otherwise known in the art to facilitate purification or to produce therapeutic conjugates (see “Antagonistic FGFR2 polypeptide conjugates,” below).

Once isolated, an anti-FGFR2 biparatopic antibody, or antigen-binding fragments thereof can, if desired, be further purified, e.g., by high performance liquid chromatography (see, e.g., Fisher, Laboratory Techniques in Biochemistry and Molecular Biology (Work and Burdon, eds., Elsevier, 1980); incorporated herein by reference), or by gel filtration chromatography, such as on a Superdex™ 75 column (Pharmacia Biotech AB, Uppsala, Sweden).

### Therapeutic Methods

Biparatopic antibodies identified as binding to and antagonizing an FGFR2 polypeptide are useful for preventing or ameliorating CCA.

In one therapeutic approach, an antibody identified as described herein is administered to a subject, such administration may be local or systemic. The dosage of the administered agent depends on a number of factors, including the size and health of the individual patient. For any particular subject, the specific dosage regimes should be adjusted over time according to the individual need and the professional judgement of the person administering or supervising the administration of the compositions.

In one embodiment, an antibody of some aspects and embodiments herein is administered in combination with a small molecule inhibitor of an FGFR. In one embodiment, the small molecule inhibitor is pemigatinib or NVP-BGJ398.

### Pharmaceutical Formulations

Also provided herein is a simple means for identifying biparatopic antibodies capable of binding to and antagonizing a polypeptide of interest (e.g., FGFR2). For therapeutic uses, the antibodies identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. An agent of some aspects and embodiments herein is administered at a dosage that blocks ligand binding to a receptor and/or that inhibits receptor activity.

### Formulation of Pharmaceutical Compositions

The administration of a biparatopic antibody may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to some aspects and embodiments herein may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to some aspects and embodiments herein may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Biparatopic Antibodies that Recognize FGFR2 Extracellular Epitopes

The aspects and embodiments as described herein are based, at least in part, on the discovery that biparatopic antibodies capable of antagonizing FGFR2 can be used as therapeutic agents to directly treat cancers, such as CCA. These therapeutic activities can be caused, for instance, by the binding of the antibody to two epitopes expressed on the surface of a cell, such as a cancer cell, and subsequently blocking FGFR ligand binding and/or inhibiting FGFR2 activity, thereby inhibiting proliferation or inducing cell death. In some embodiments, a biparatopic antibody is used to enhance antibody derived cellular cytotoxicity (ADCC) in a cancer cell.

The practice of aspects and embodiments herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of some aspects and embodiments herein, and, as such, may be considered in making and practicing some of the aspects and embodiments herein. Particularly useful techniques for particular embodiments will be discussed in the sections that follow.

The following examples are put forth so as to provide those of ordinary skill in the art with a complete disclosure and description of how to make and use the assay, screening, and therapeutic methods of some aspects and embodiments herein, and are not intended to limit the scope of the various aspects and embodiments herein.

### EXAMPLES

### Example 1: Targeting FGFR2 in Cholangiocarcinoma (CCA)

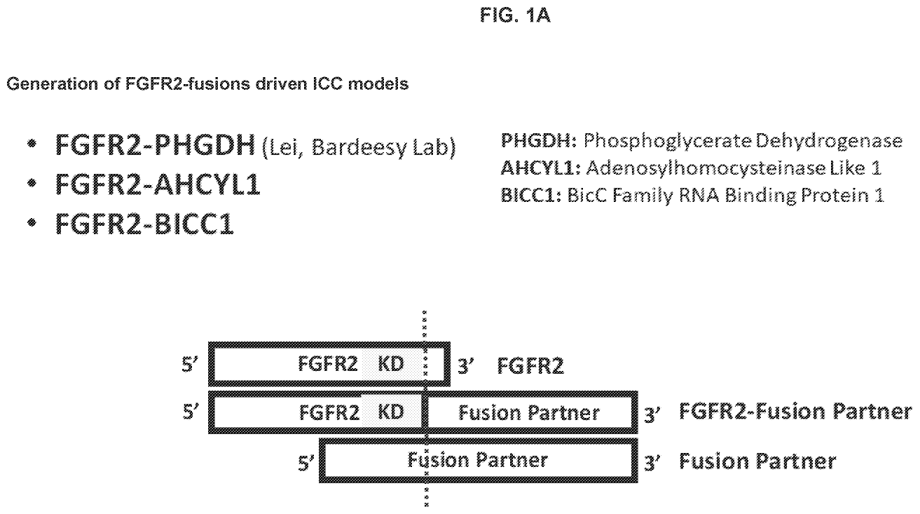

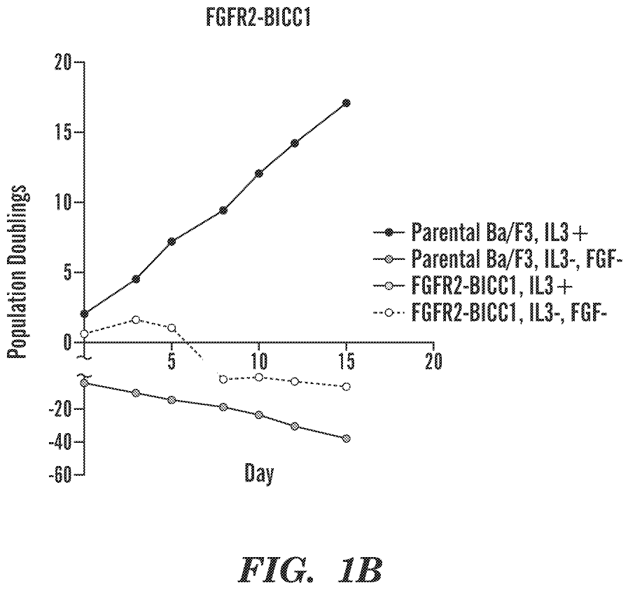

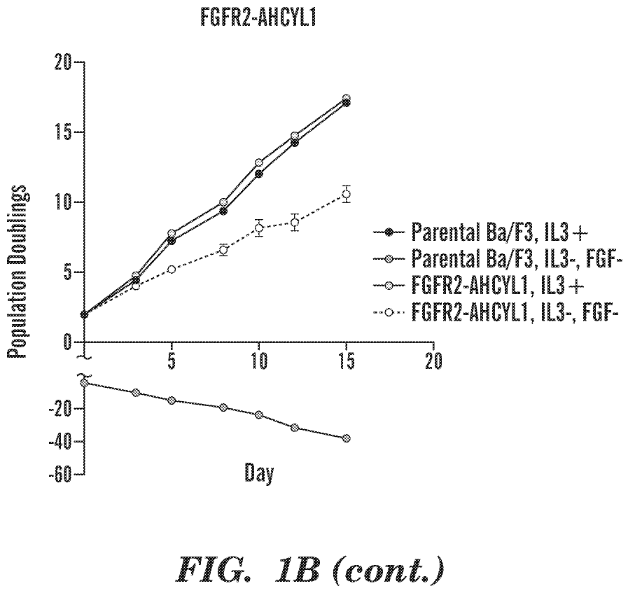

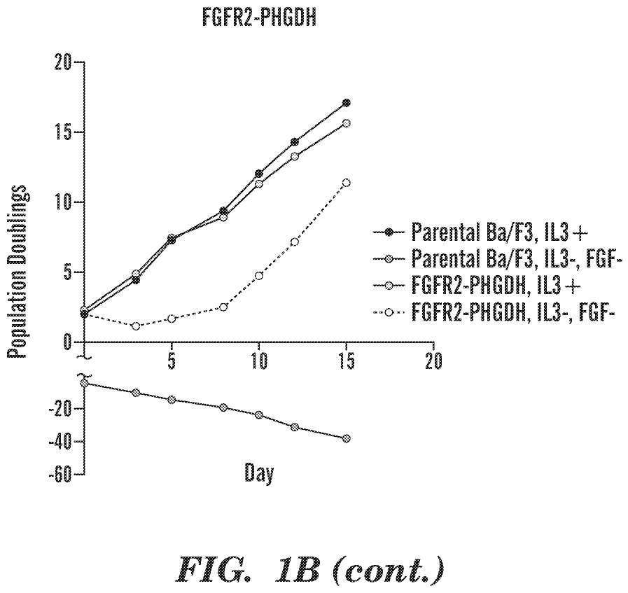

Genomic changes that result in the fusion of FGFR2 to another protein are common in CCA. To facilitate analysis of FGFR2 fusions driving intrahepatic CCA, the following FGFR2 fusions were generated: FGFR2-Phosphoglycerate Dehydrogenase (PHGDH), FGFR2-Adenosylhomocysteinase Like 1 (AHCYL1), and FGFR2-BICC1 (FIG. 1A). The effect of FGFR2 fusions was analyzed on cell proliferation (FIG. 1B). The FGFR2-AHCYL1, FGFR2-PHGDH fusions transformed BaF3 cells in IL3 depletion assays (FIG. 1B). FGFR2-BICC1 also partially transformed BaF3 cells.

### Example 2: FGFR2 Fusion Expression was Sufficient for Transformation

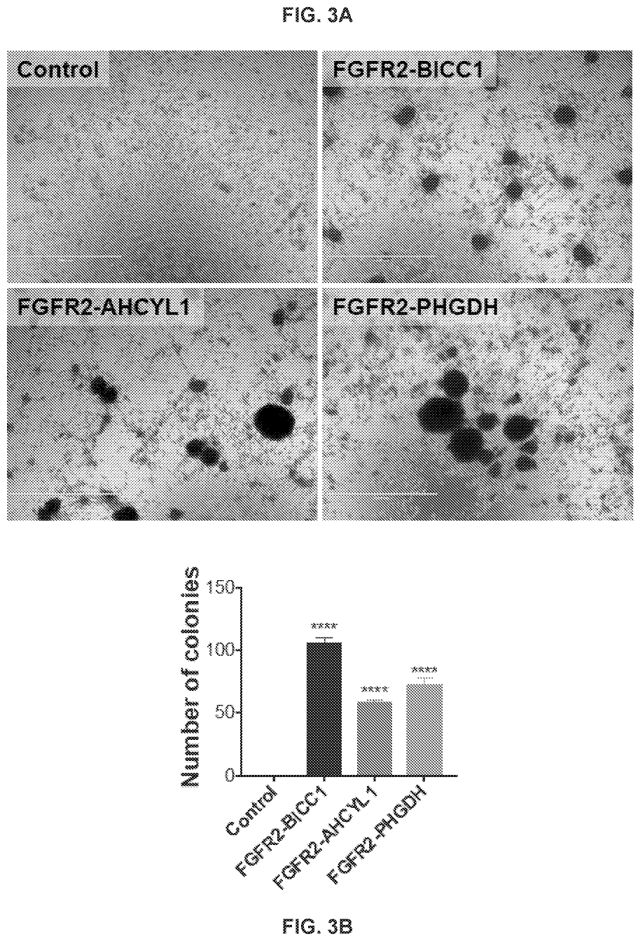

The effect of FGFR2 fusion expression on NIH3T3 cells was also tested. Interestingly, FGFR2 fusions were sufficient to transform the cells as shown in focus formation assays. NIH3T3 cells expressing FGFR2-AHCYL1, FGFR2-PHGDH, and FGFR2-BICC1 all formed colonies in culture, which is indicative of oncogenic transformation (FIG. 3A). FIG. 3B quantifies the number of colonies present in cultures of NIH3T3 cells expressing the FGFR2 fusions. This data demonstrates that expression of the FGFR2-fusions was sufficient to transform NIH3T3 cells.

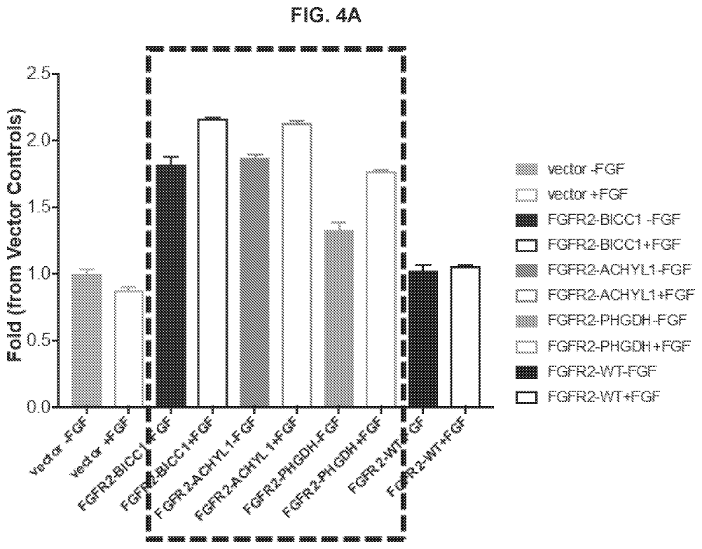

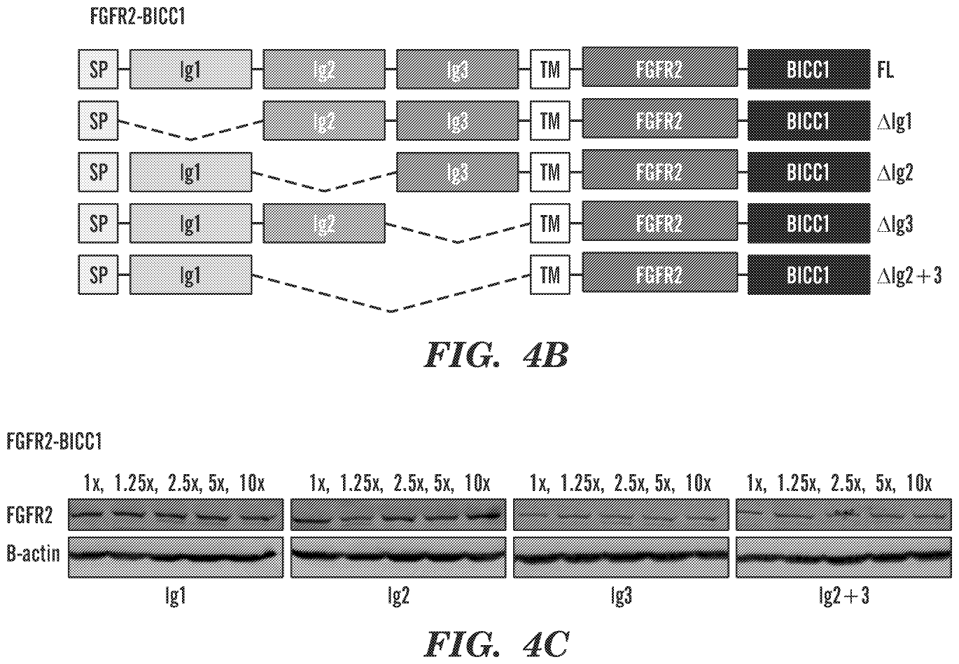

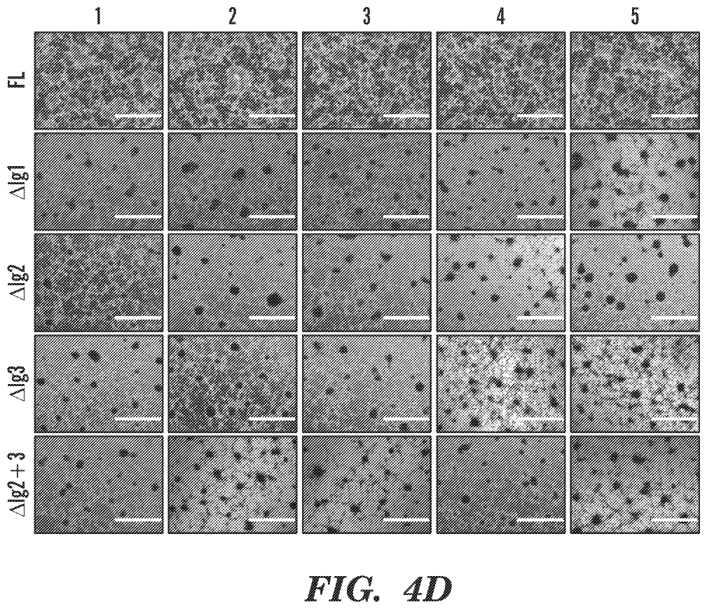

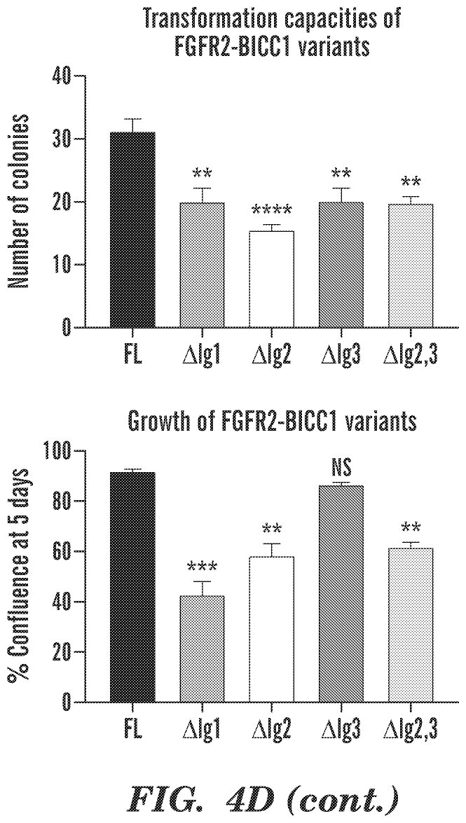

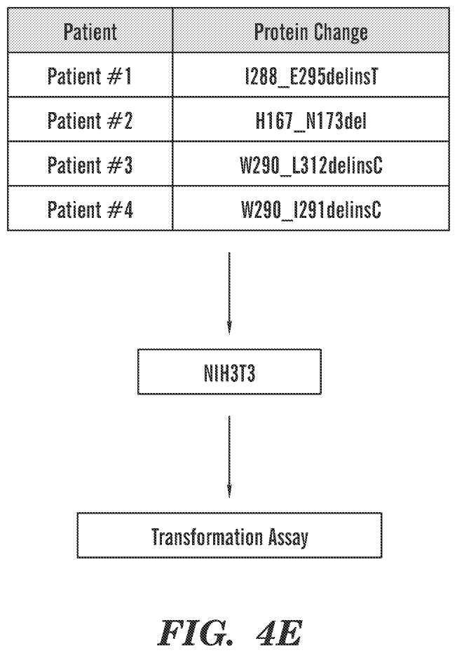

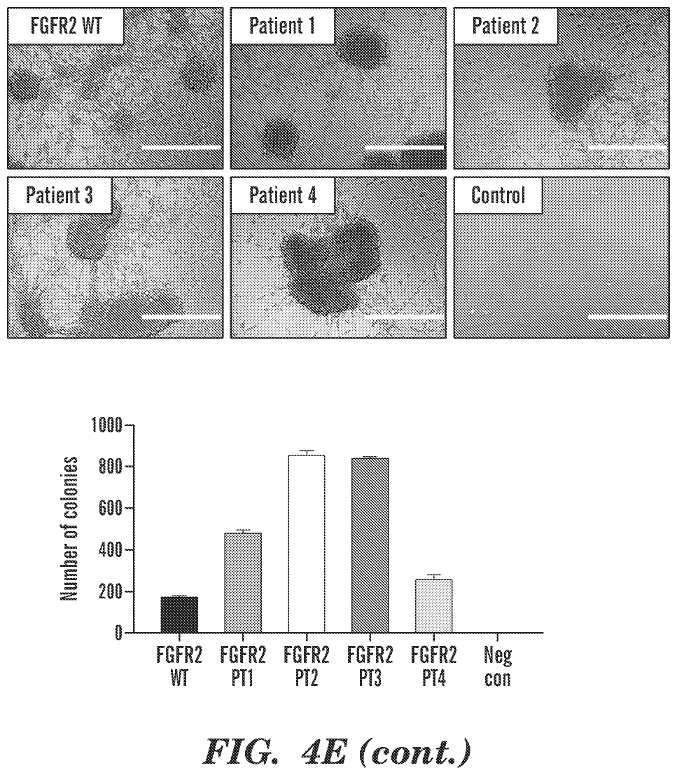

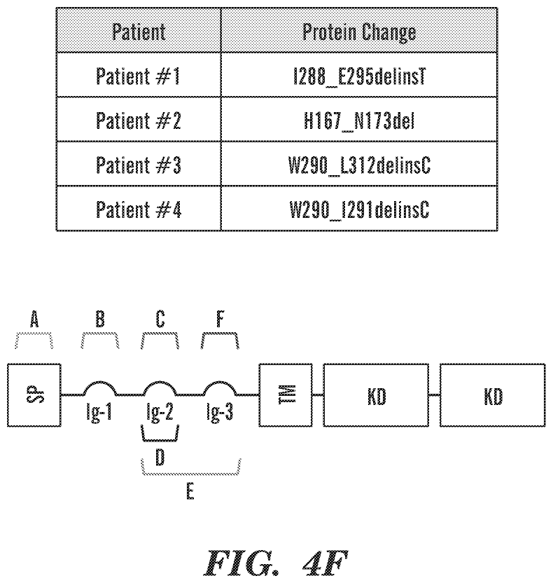

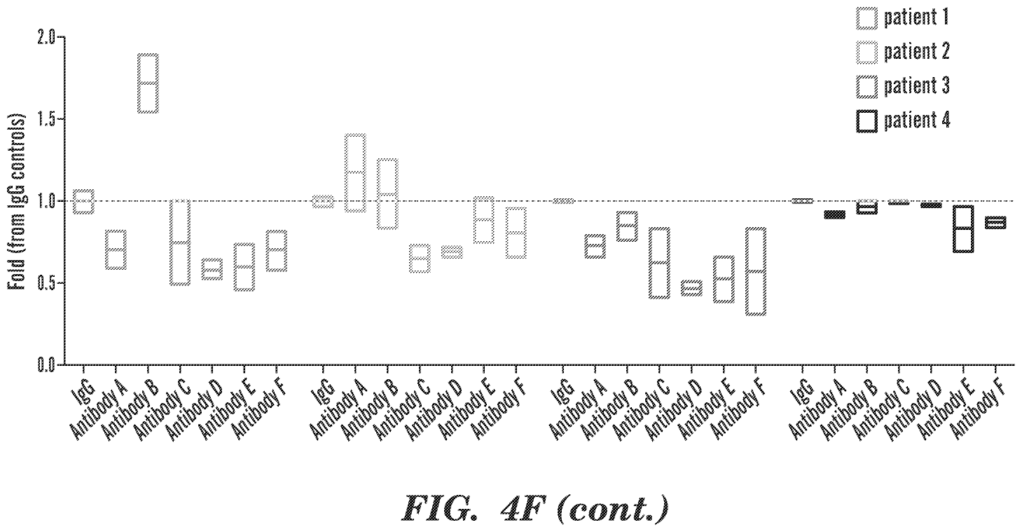

Expression of FGFR2-BICC1, FGFR2-AHCYL1, and FGFR2-PHGDH in NIH3T3 cells induced increased proliferation. Cells expressing the FGFR2 fusions grew faster than control cells, and this response was enhanced in the presence of FGF ligands (FIG. 4A). The effects of deleting the Ig-like extracellular domains of the FGFR2-BICC1 fusion on cell transformation and growth is shown in FIGS. 4B-4D. In FIGS. 4E and 4F, CCA Patients 1-4 had mutations in the ECD of FGFR2. In particular, for CCA Patients 1, 3, and 4, the FGFR2 mutation was in domain IgIII of the ECD, and for Patient 2, the FGFR2 mutation was in domain IgII of the ECD. Such mutations in the FGFR2 ECDs can increase transformation of cells. As shown in FIG. 4E, the CCA patient-derived FGFR2 extracellular domain mutations increased transformation capacity. As demonstrated in FIG. 4F, some parental antibodies were shown to be effective in inhibiting or blocking patient-derived FGFR2 ECD mutation driven cell growth. By way of example, antibodies C, D, and E were effective in blocking the growth of cells having a patient-derived FGFR2 ECD activating mutation, as determined by assessing fold-increase or decrease from a nonspecific Ab (IgG) control (FIG. 4F); therefore, combinations of the binding regions of these antibodies in biparatopic antibodies may likely result in a synergistic blocking effect.

### Example 3: Inhibition of FGFR2 Fusions

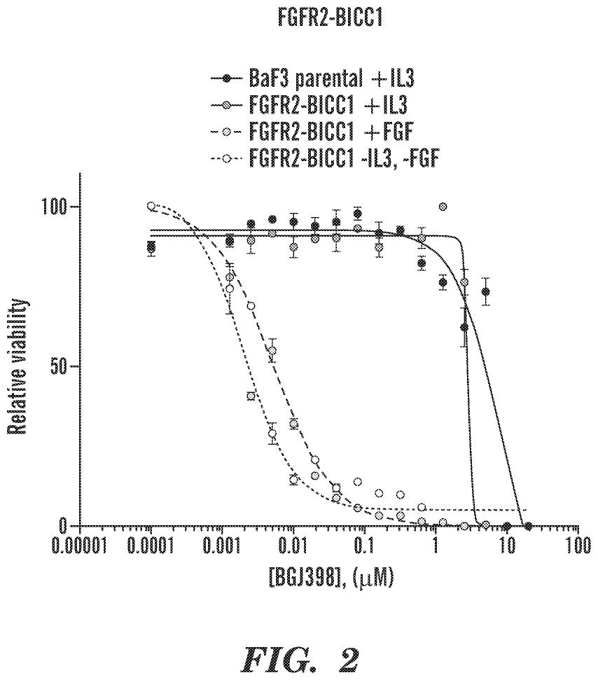

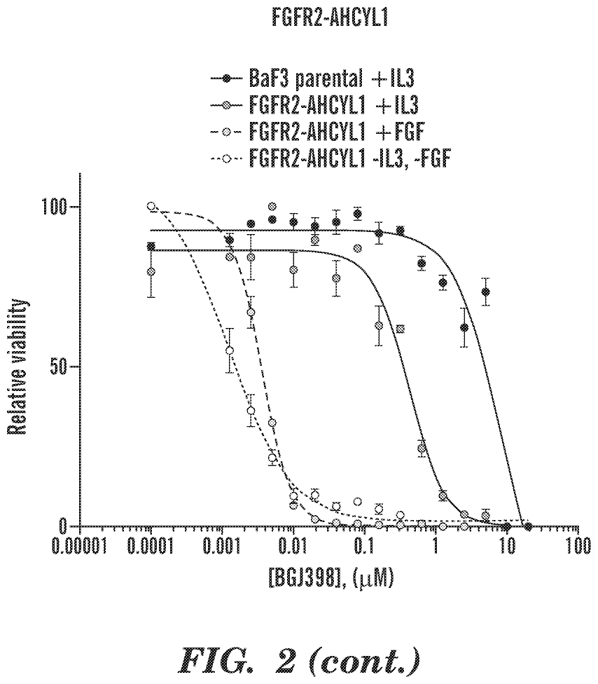

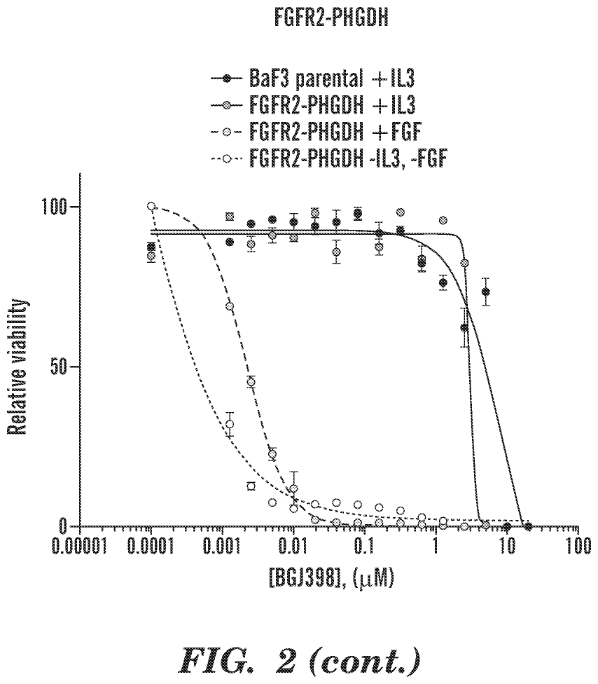

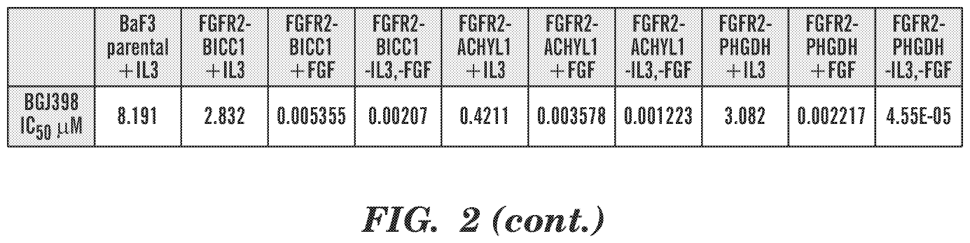

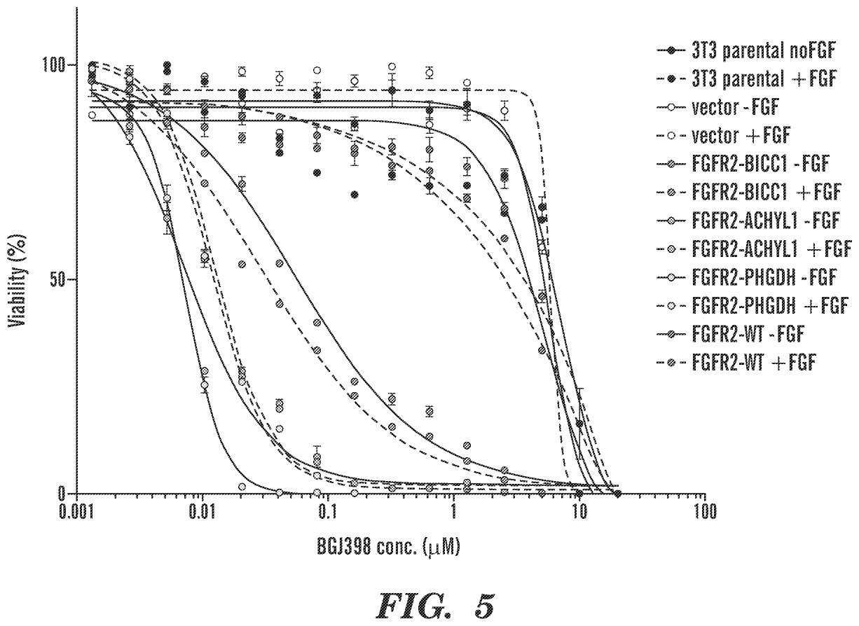

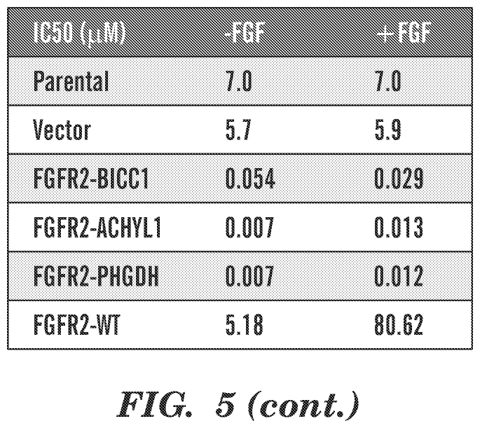

BGJ398 (also termed NVP-BGJ398) is a small molecule inhibitor of FGFR2, which displayed encouraging efficacy in patients with FGFR2 fusion-positive ICC in a phase II trial. The inhibitory activity of NVP-BGJ398 was tested against BaF3 cells expressing FGFR2 fusions. Interestingly, the cells showed sensitivity to the FGFR2 inhibitor in viability assays (FIG. 2). NIH3T3 cells transformed with FGFR2-BICC1, FGFR2-AHCYL1, and FGFR2-PHGDH fusions were also sensitive to BGJ398 treatment (FIG. 5). These results indicate that these fusions signal via FGFR.

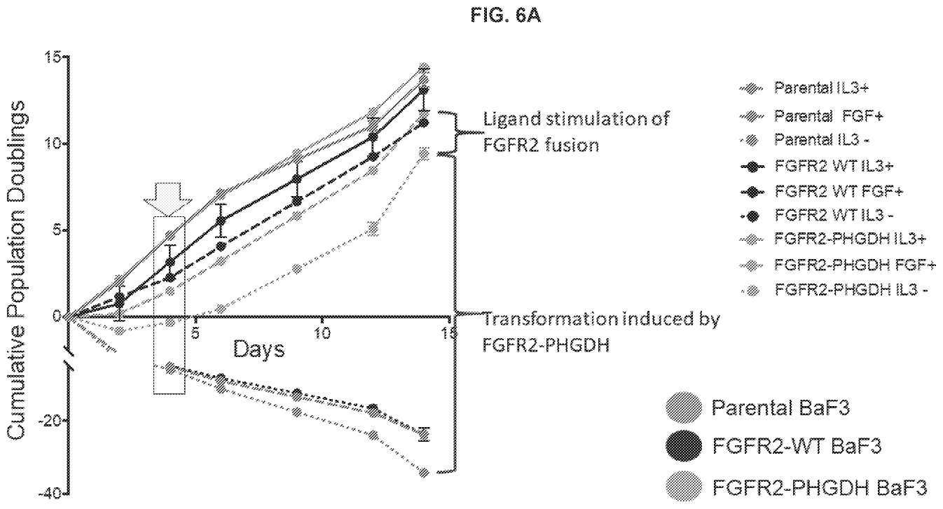

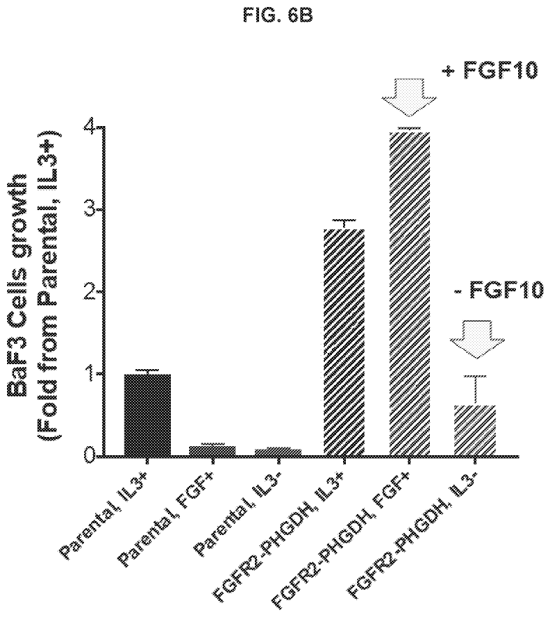

The FGF ligand, FGF10, augmented the growth of FGFR2-PHGDH fusion expressing BaF3 cells (FIG. 6A). This effect was quantified in an IL3 depletion assay (FIG. 6B). The ability of cells to grow in the absence of IL3 indicates the “transforming capacity”. In FIG. 6B parental cells in +IL3 is the control and the growth of FGFR2-PHGDH is measured as fold difference from control. BaF3 parental cells in the absence of −IL3 die, while FGFR2-PHGDH expressing cells continue to grow in −IL3 condition, indicating that FGFR2-PHGDH transformed BaF3 cells.

### Example 4: Design of Biparatopic Antibodies

As detailed herein above, BGJ398 is capable of inhibiting FGFR2. This inhibition was effective in reducing the oncogenic effects of FGFR2 fusion expression in a variety of cell types. Biparatopic antibodies that bind and antagonize the FGFR2 receptor are expected to be useful for the treatment of cancer (e.g., CCA). Such antibodies can be tested for anti-oncogenic activity in viability assays, binding assays, and dimerization assays in BaF3 and NIH3T3 cells transformed by FGFR2 fusion expression.

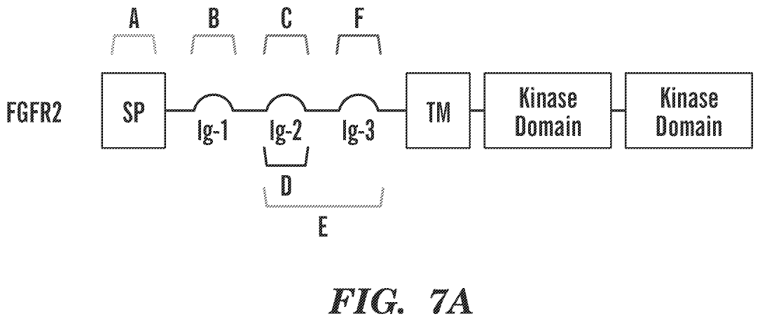

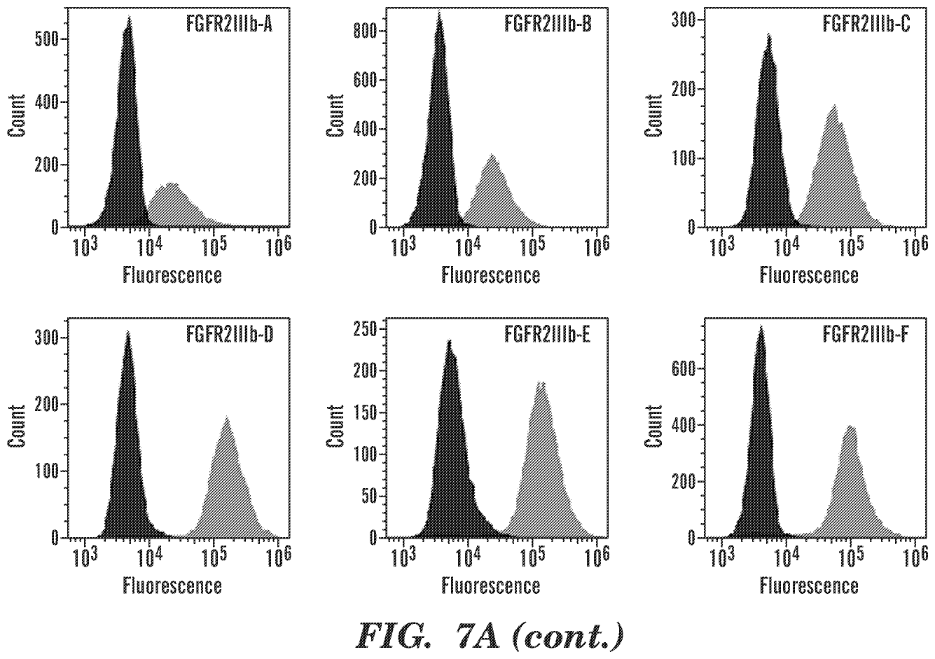

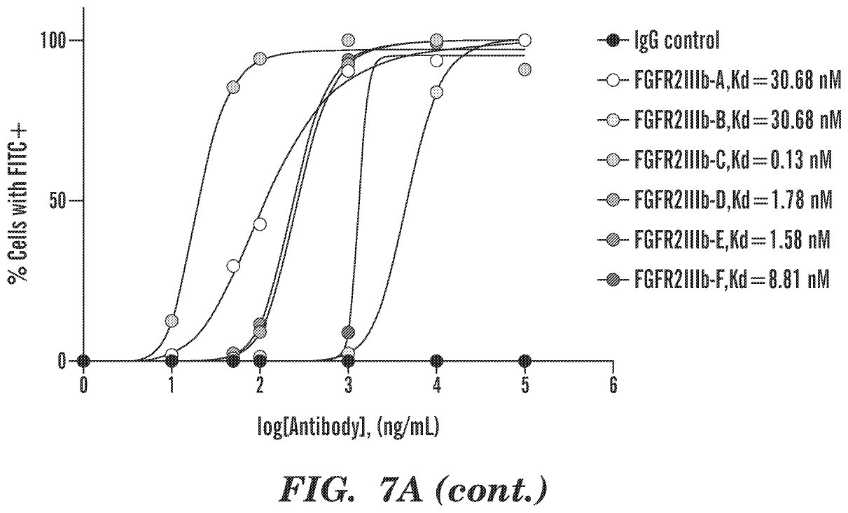

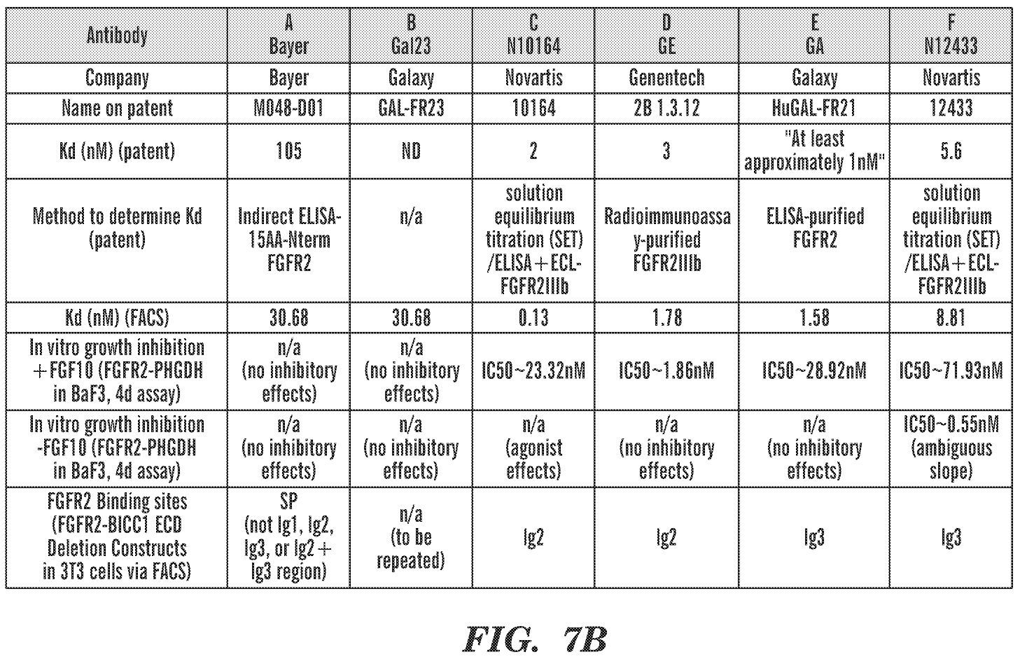

In designing biparatopic antibodies, Applicants have focused on antibodies that specifically bind the FGFR2 extracellular domain, which contributed to the growth of cells transformed by expression of FGFR2 fusions. Antibodies A-F are commercially available antibodies that are described in the patent literature (FIG. 7B), and that bind epitopes present in the extracellular domain of FGFR2 (FIG. 7A).

Regions of the FGFR2 extracellular domain bound by the antibodies include the signal peptide (SP) and three immunoglobulin-like domains (IgI, IgII and IgIII). Antibodies A-F were used in FACS analysis of a SNU-16 gastric cancer cell line with FGFR2 amplification (FIG. 7A, 7B) At the right of the figure, the percent of cells that were GFP positive is shown as a function of the log of antibody concentration for antibodies A-F against the various FGFR2 domains shown in the schematic. These results demonstrate that antibodies A-F all specifically bound cells expressing FGFR2.

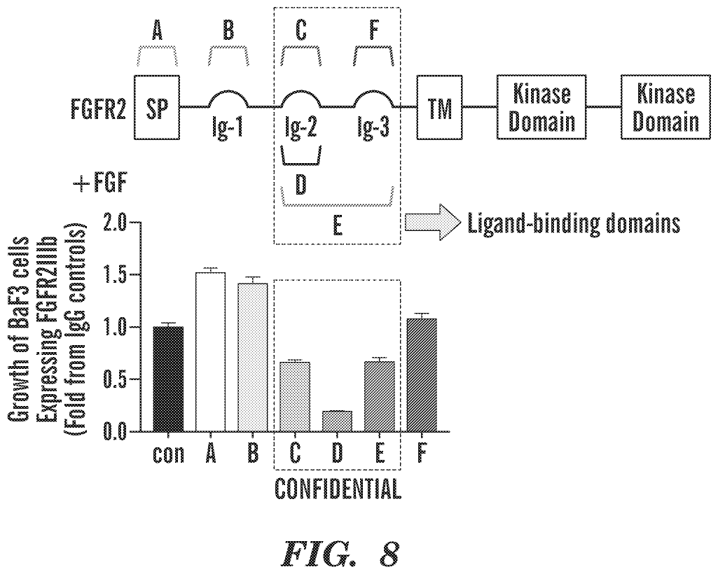

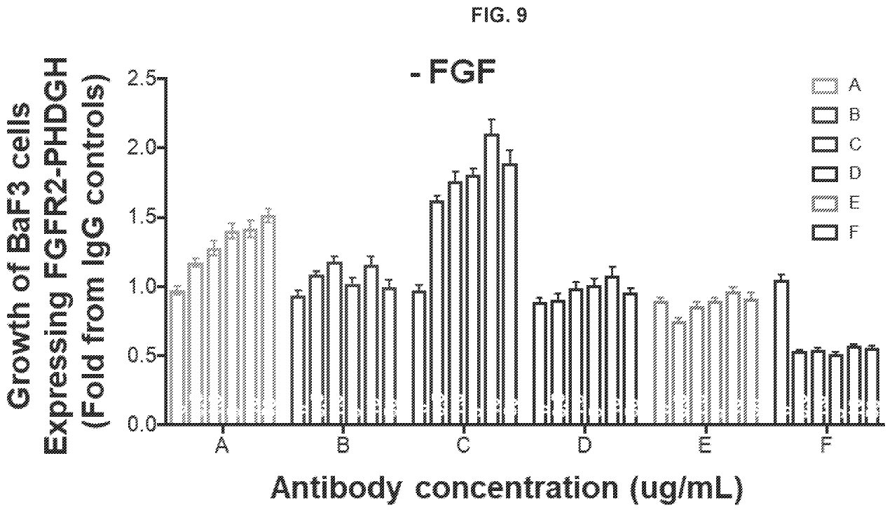

Antibodies A-F were tested to determine whether they could inhibit the ligand-induced growth of BaF3 cells expressing FGFR2IIIb. Antibodies C, D, and E, which bind FGFR2 Ig-2 and Ig-3, were effective in blocking ligand-induced growth of BaF3 cells expressing wild-type FGFR2IIIb (FIG. 8). The effects of antibodies A-F were then tested on BaF3 cells expressing FGFR2-PHDGH fusions. Interestingly antibodies A and C, and to a lesser extent D, all showed agonist activity in the absence of FGF ligands (FIG. 9). Antibody F inhibited the FGFR2-PHDGH activating fusion in BaF3 cells (FIG. 9).

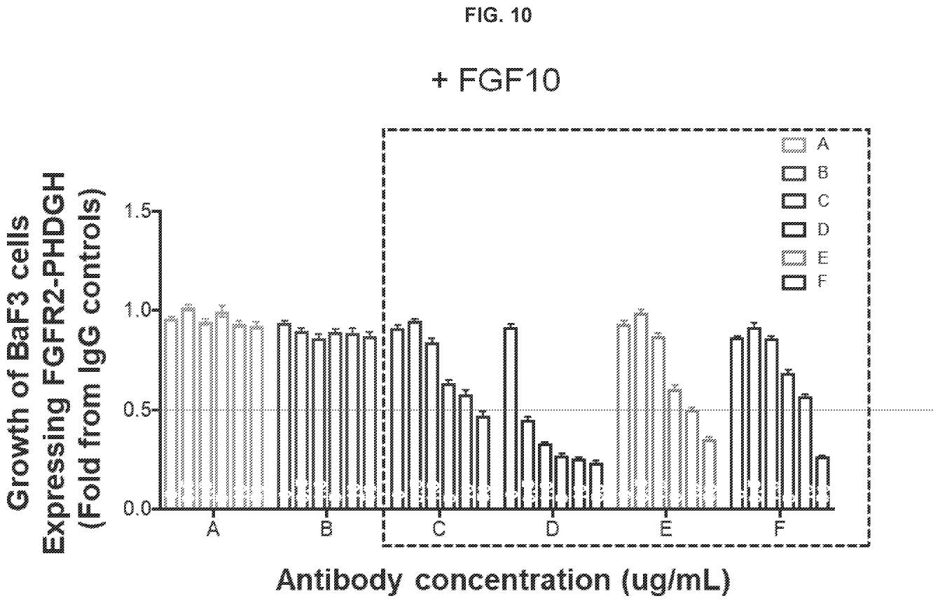

The growth of BaF3 cells expressing an FGFR2-PHDGH fusion was assayed in the presence of FGF ligand, FGF10. Antibodies C, D, E and F inhibited ligand stimulated growth of FGFR2-PHDGH fusion expressing BaF3 cells (FIG. 10). Based on the results reported herein above, antagonistic biparatopic antibodies are made using the FGFR2 antibody combinations shown in FIG. 11A. These combinations result in increased growth inhibition effects in oncogenic or cancer cells (e.g., transformed BaF3 cells) that overexpressed FGF2 fusions. Thus, antibodies that bind to epitopes have increased inhibitory and killing effects against cancer cells. Designs for antagonistic biparatopic antibodies are shown at FIGS. 11B and 11C. Advantageously, including complementary mutations in VH-VL and CH1-CL on one-half of the biparatopic antibody results in preferential heterodimeric pairing between two different chains rather than chains from the other half of the same antibody. Alternatively, a linker method is used to create scFab, scFv, or DuoBodies. In this method two antibody chains are each generated in different cells and mixed in vitro. Such methods are known in the art and described, for example, by K. Ding et al, March 2017, Appl. Microbiol. Biotechnol., 101(5):1889-1898; and by V. Jager et al., 2013, BMC Biotechnol., 13:52.

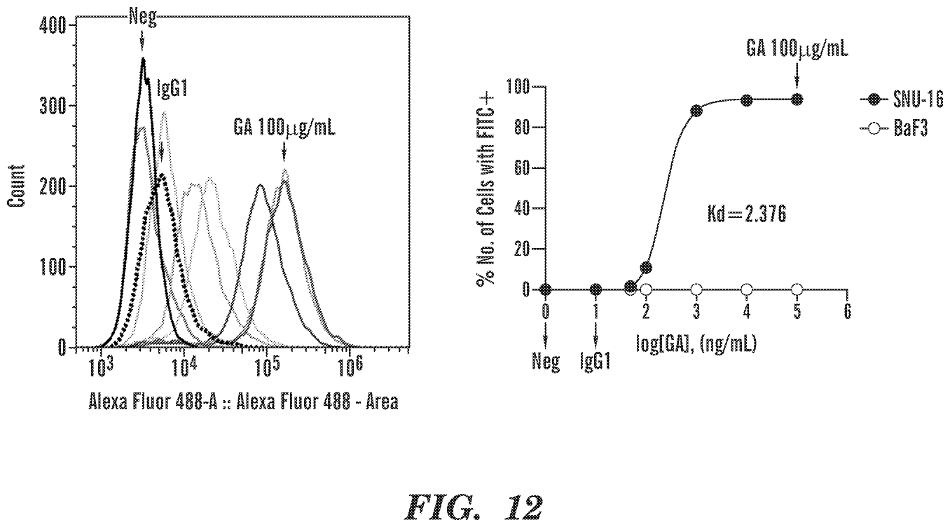

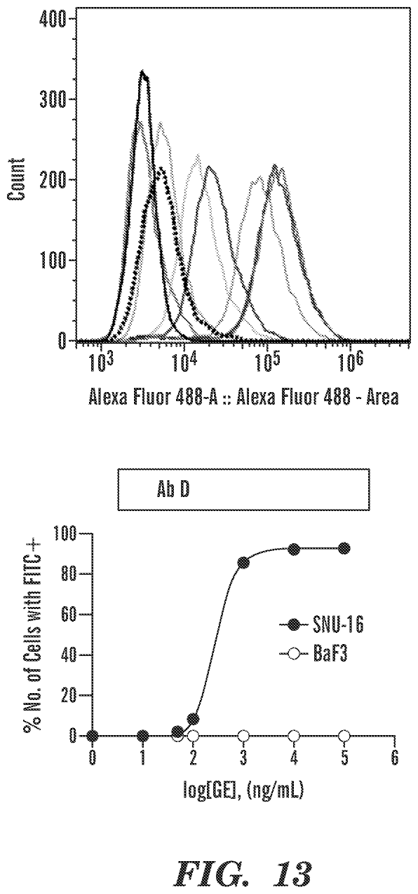

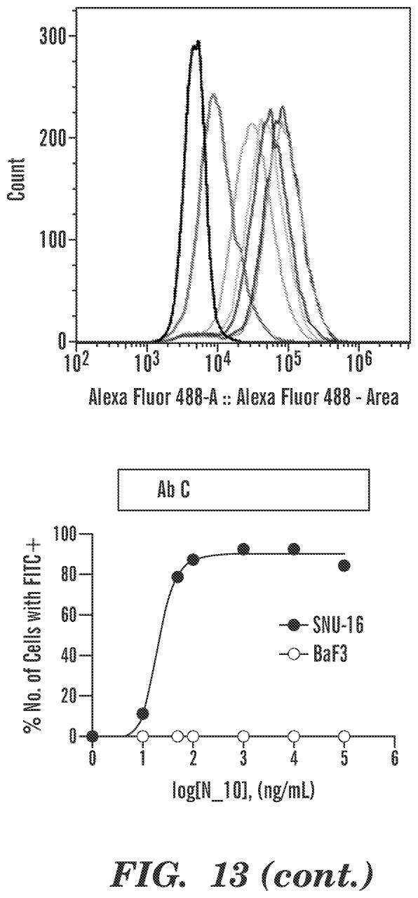

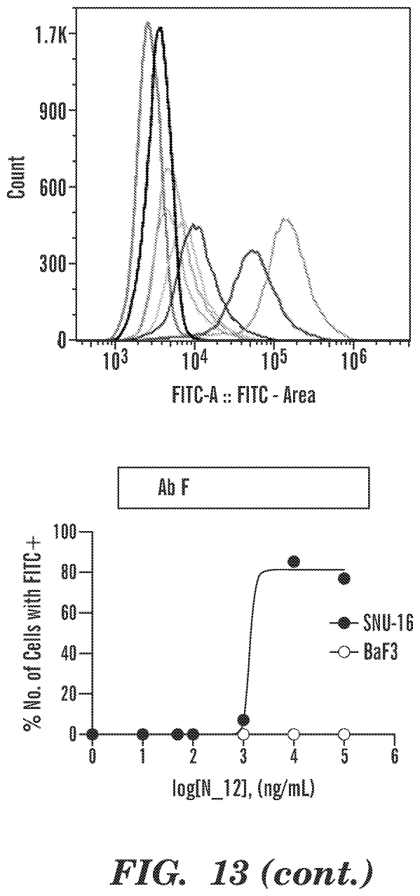

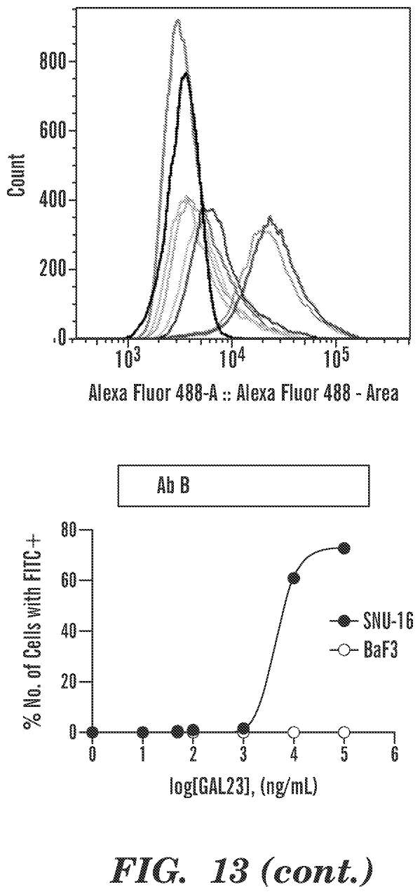

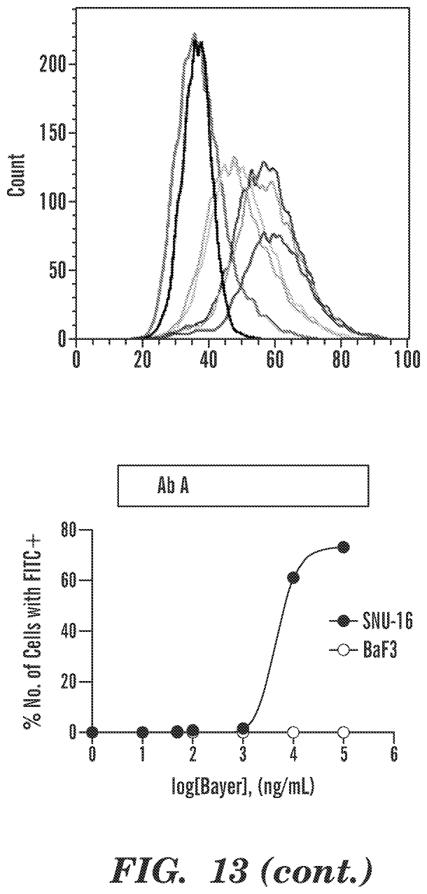

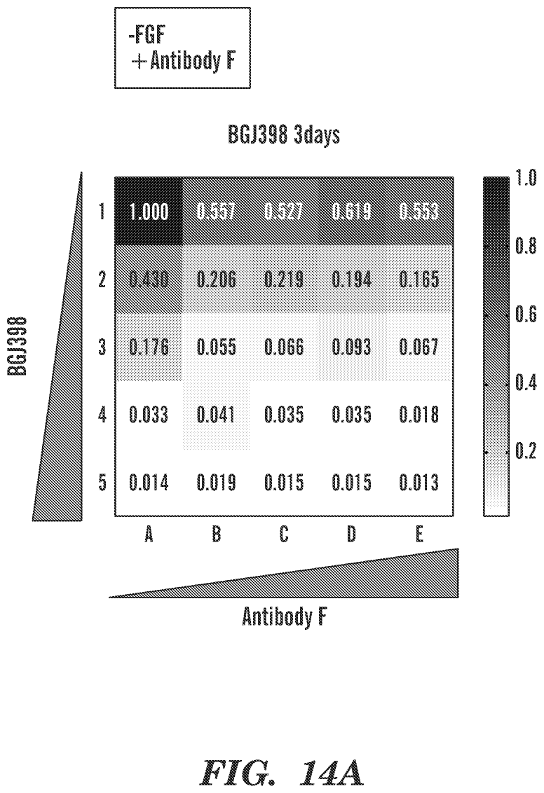

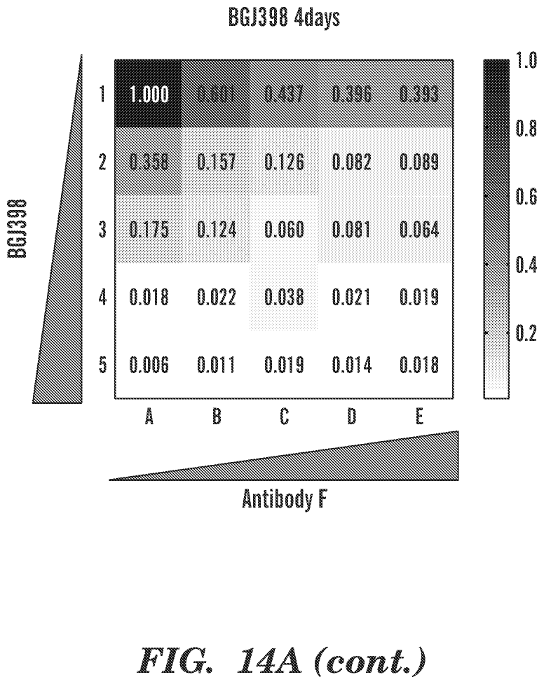

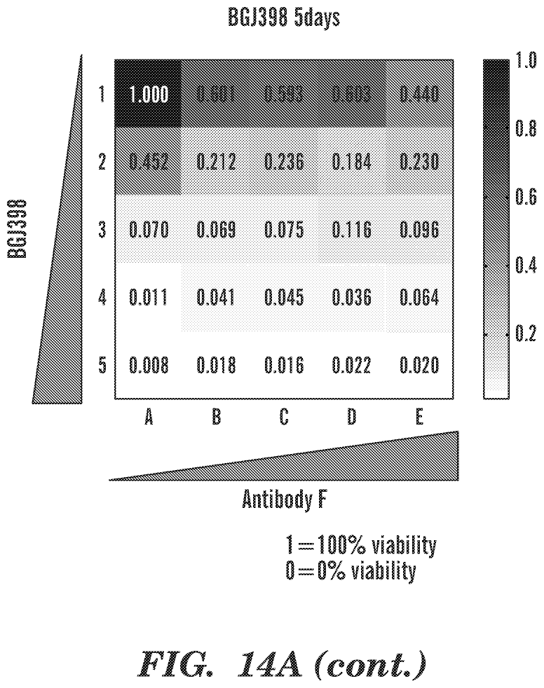

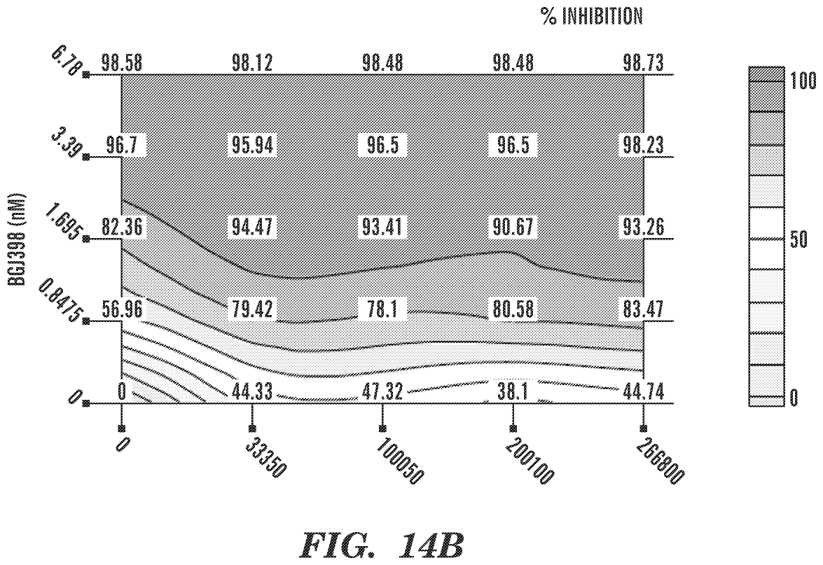

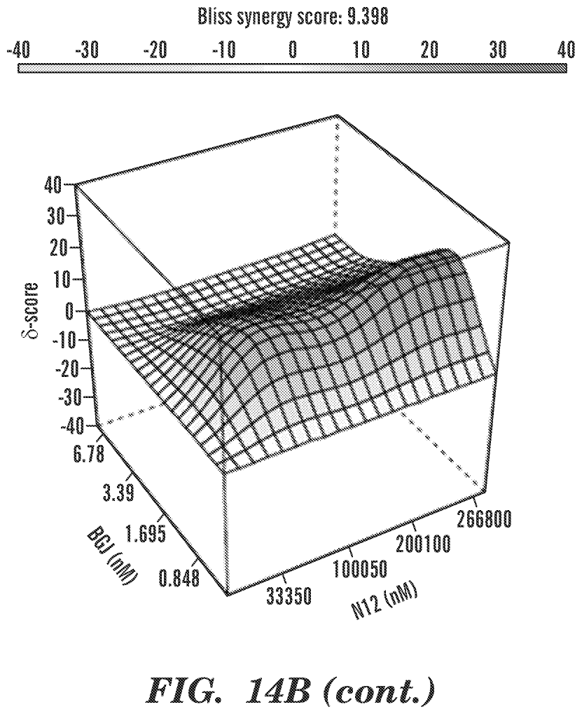

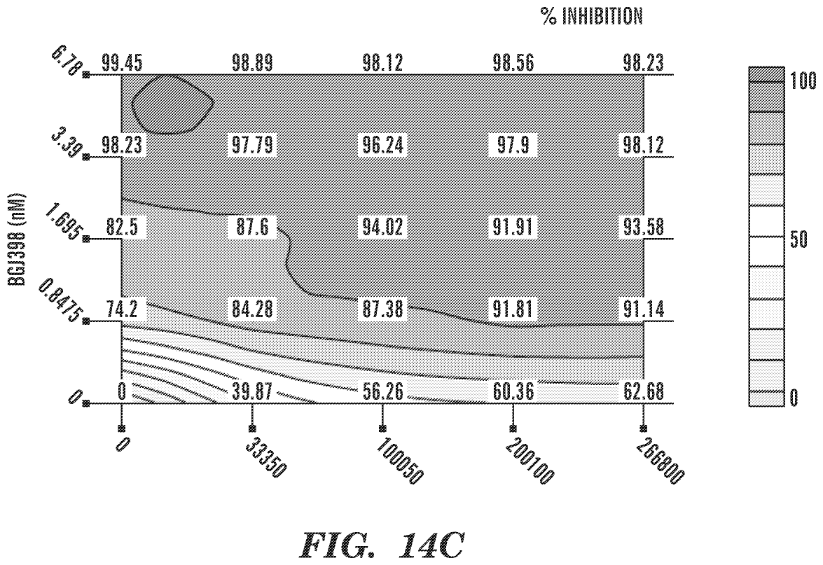

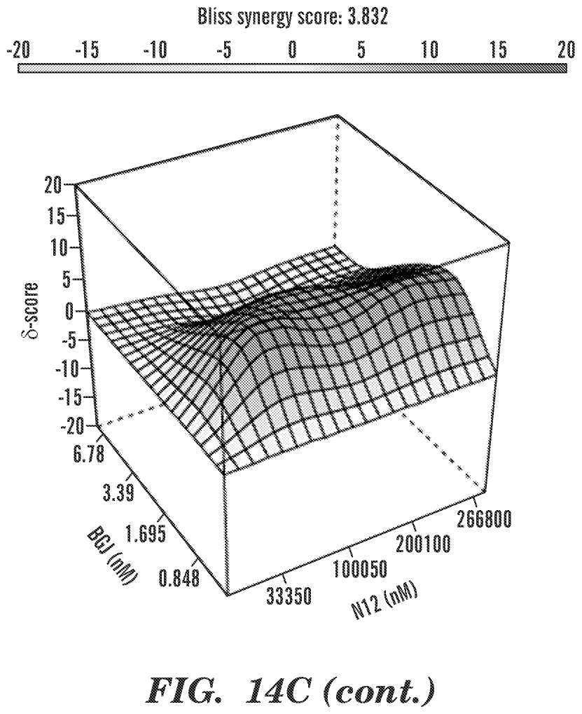

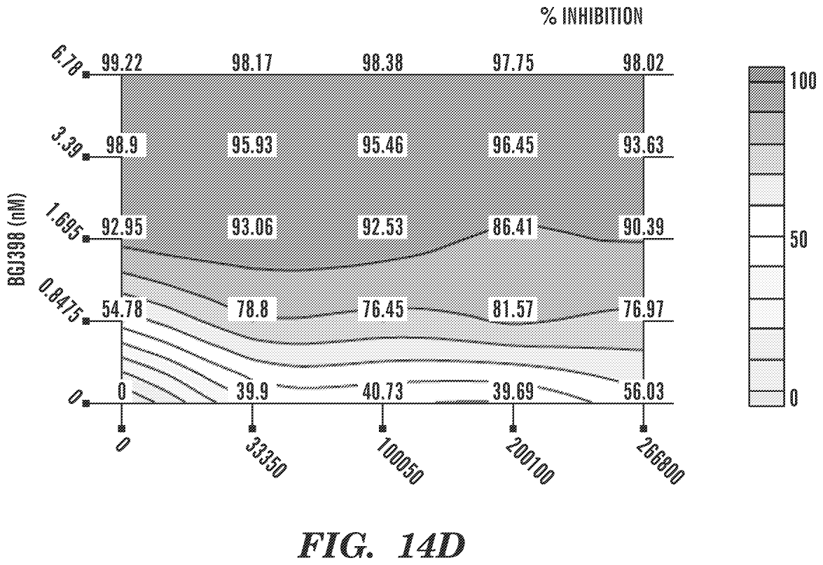

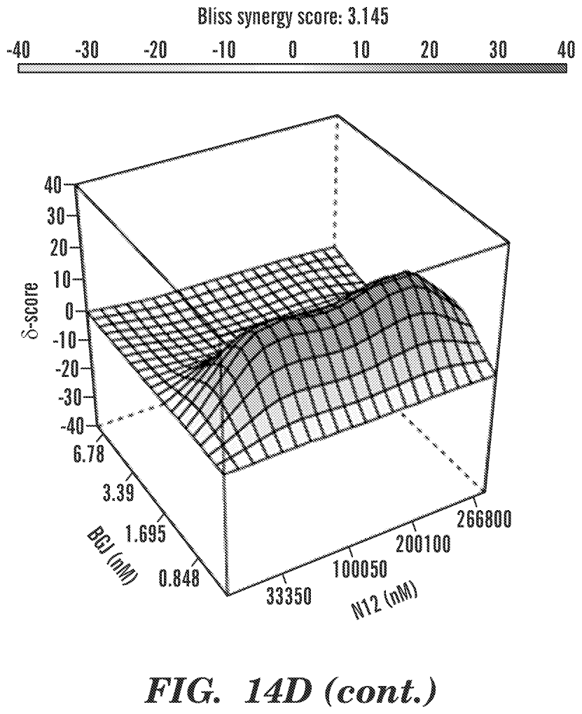

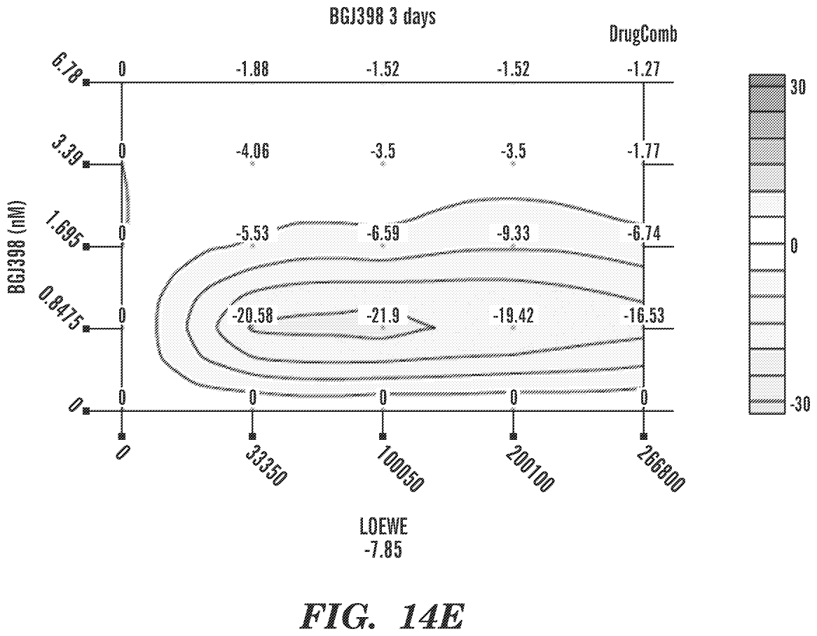

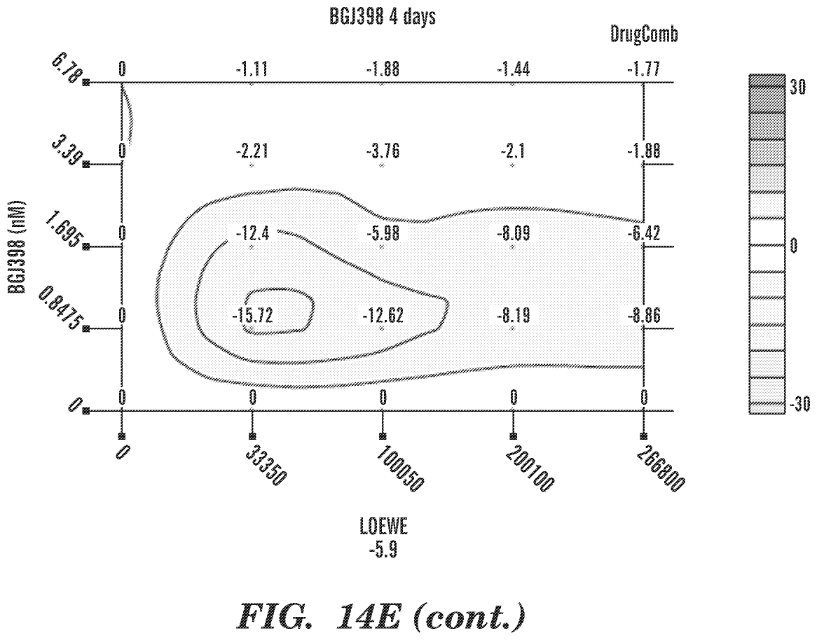

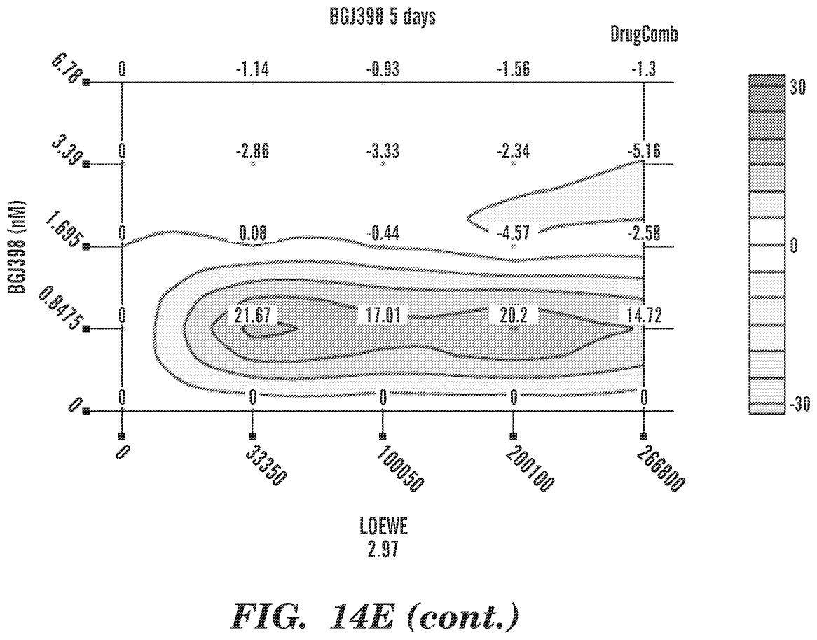

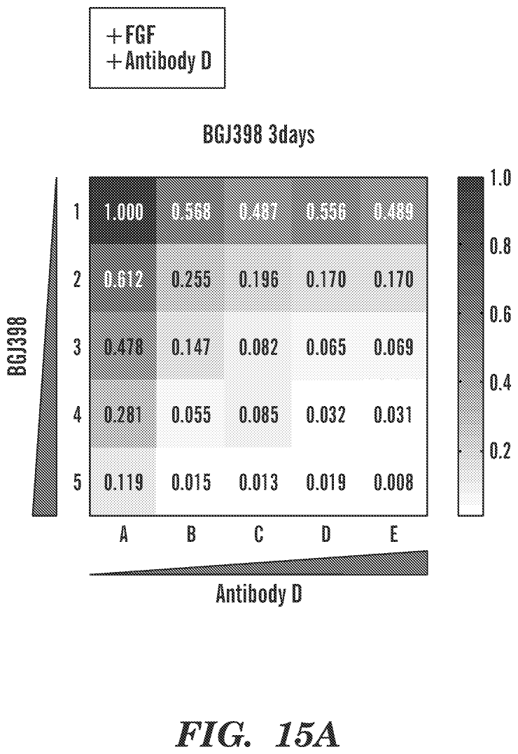

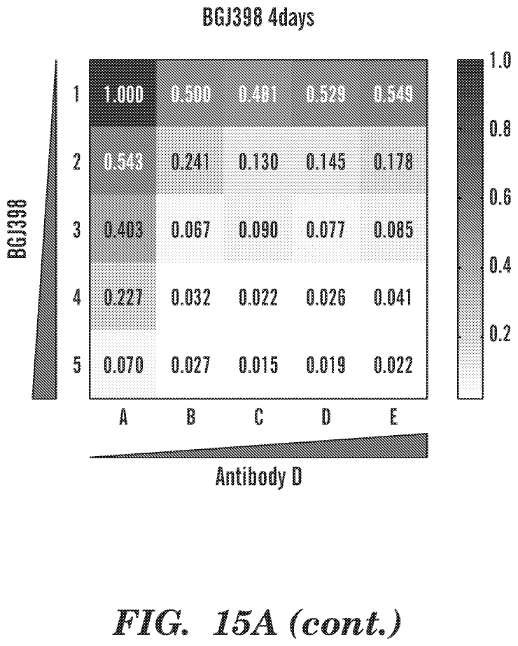

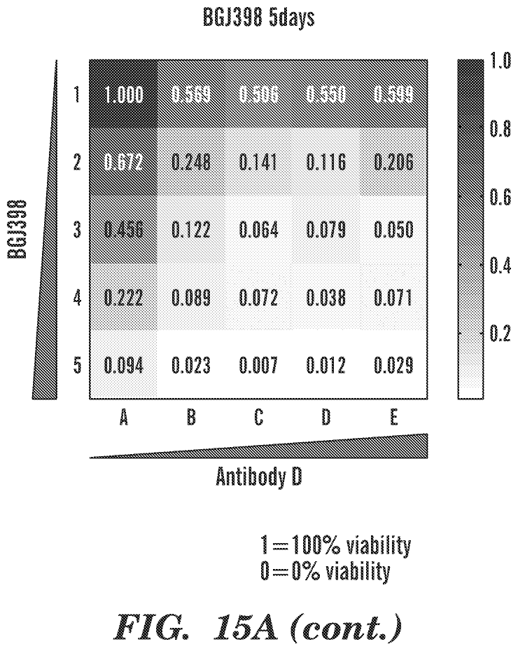

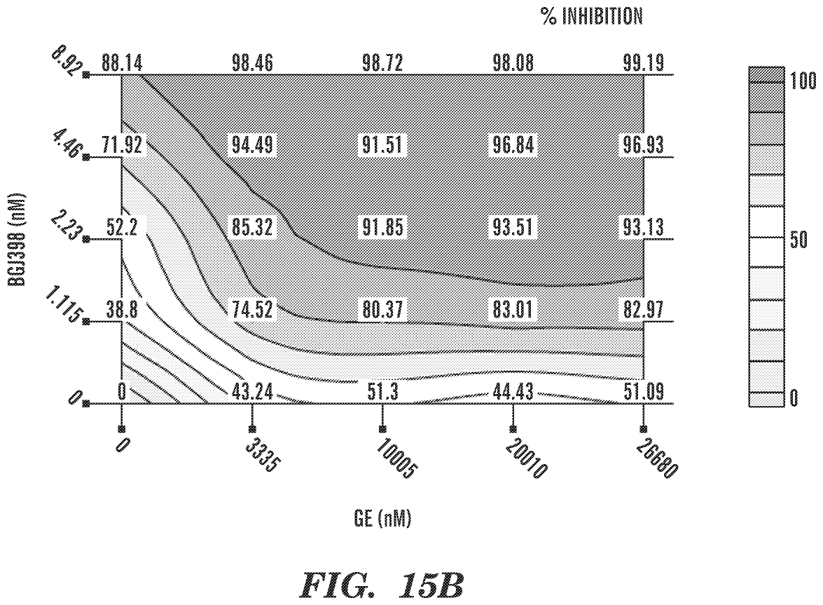

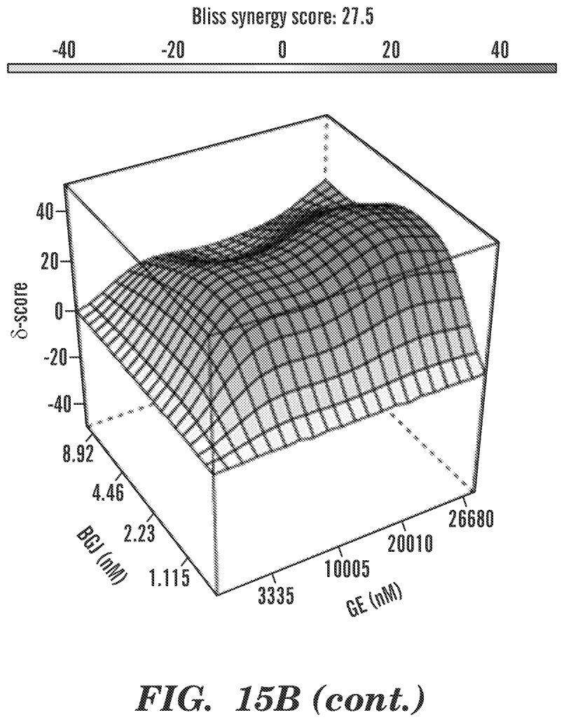

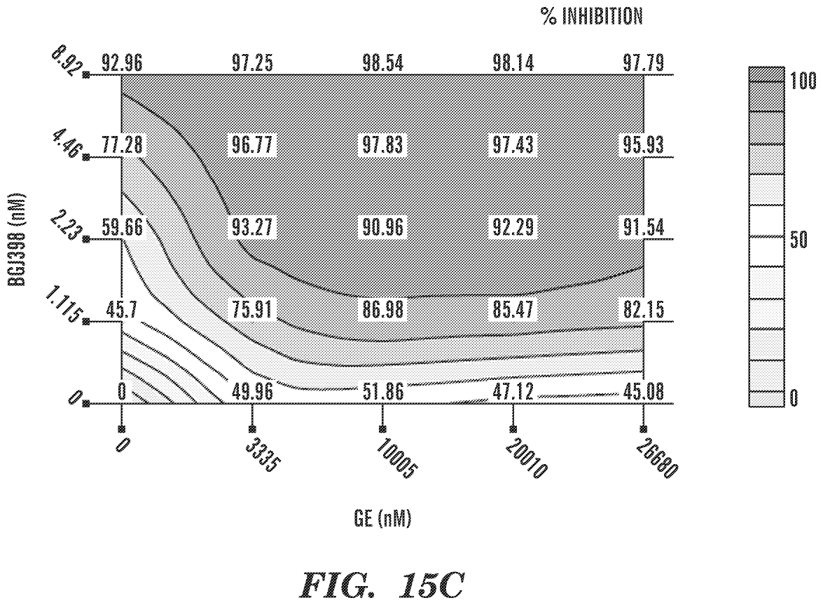

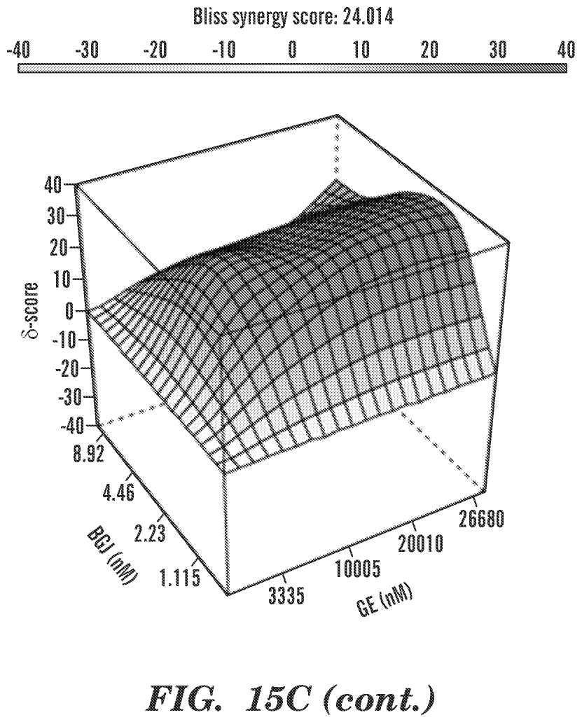

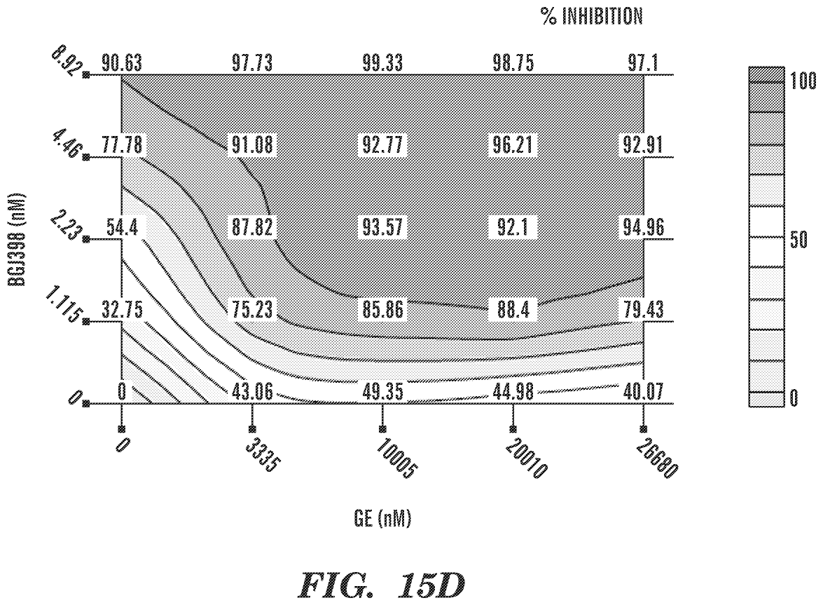

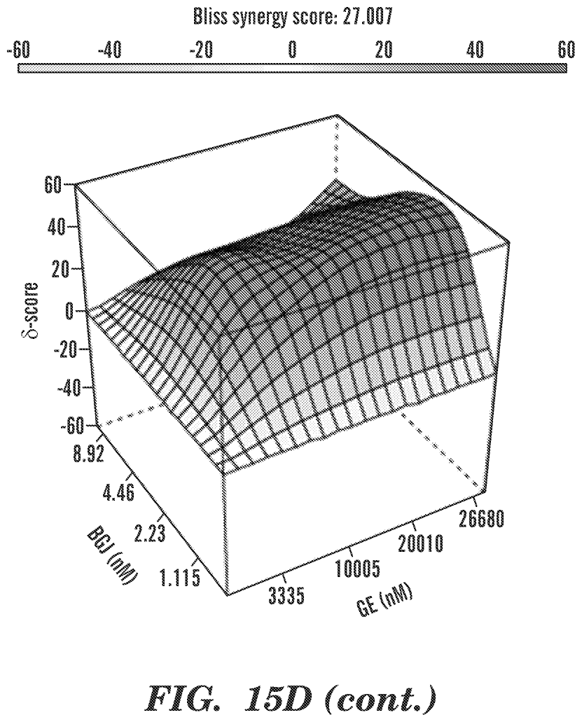

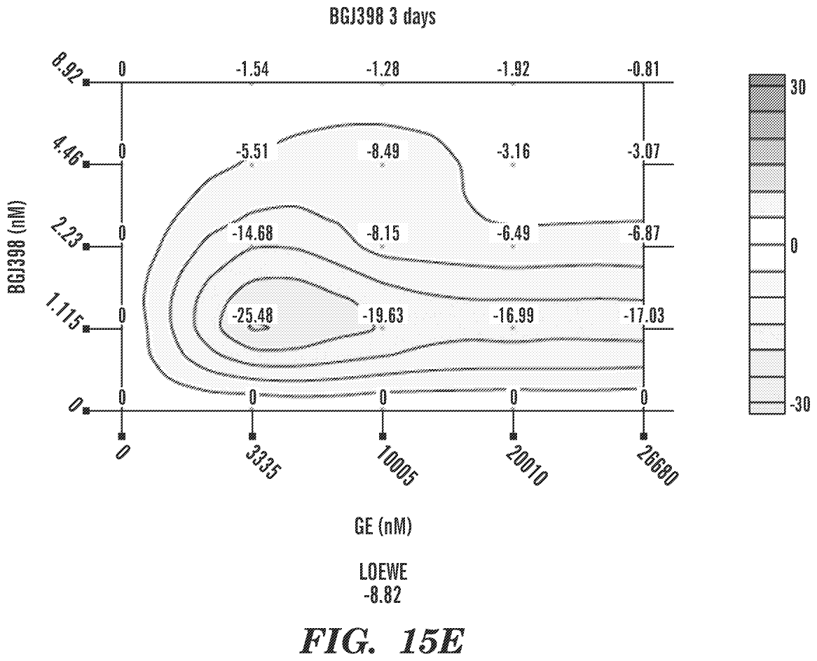

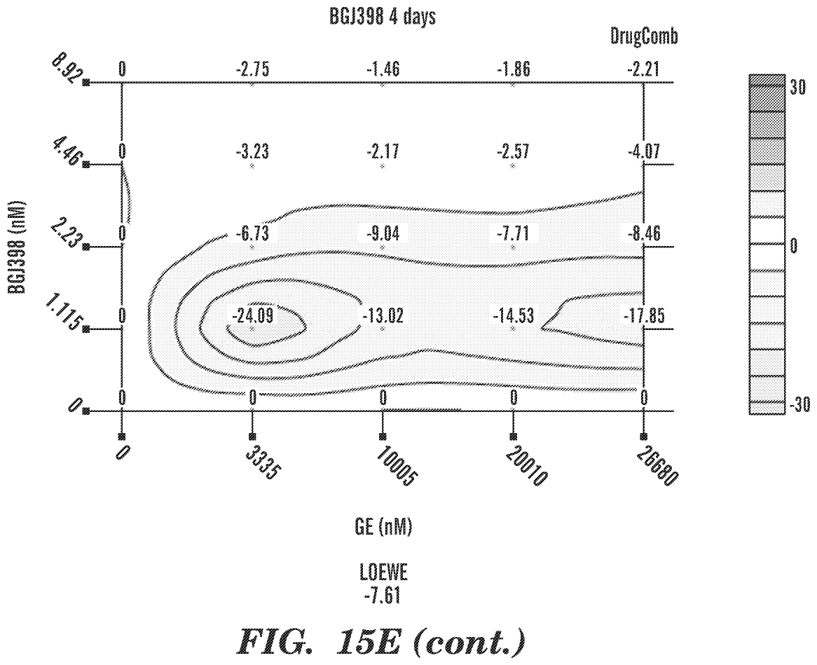

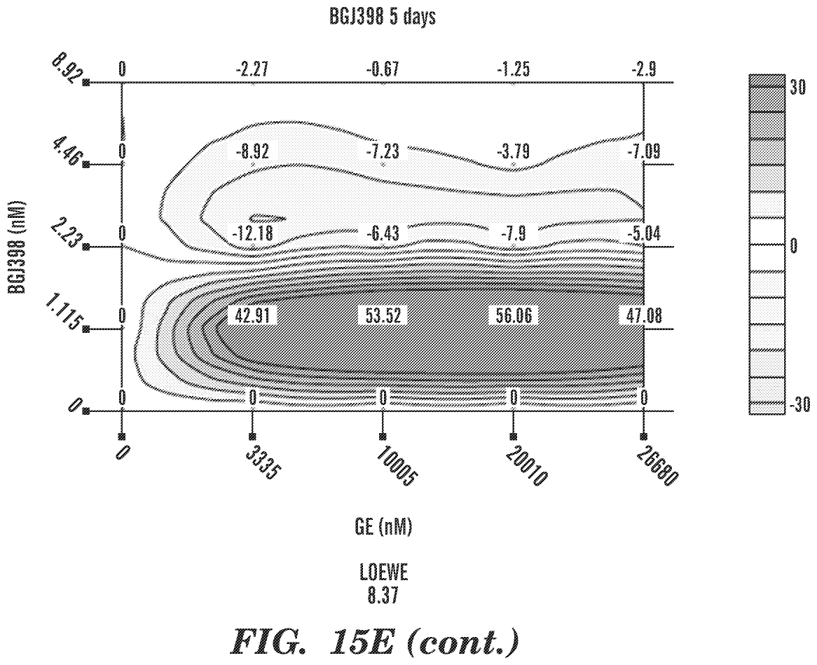

FGFR2 antibody validation was assayed using FACS analysis (FIG. 12). In a binding assay, a shift in fluorescence levels was observed in negative and IgG controls occurred when antibodies bind FGFR2 (FIG. 12). The shift increased with increased binding to FGFR2 (FIG. 12). The various curves represent the concentrations of antibodies used. The GA antibody (also termed “Antibody E”) was obtained from Galaxy. In previous studies it had a 30.68 Kd of ˜1 nM, which was found to be 1.58 nM experimentally as shown in FIG. 7B (nM) (FACS). The righthand panel showed the percentage of cells showing fluorescence at various concentrations of the GA antibody. In the graph on the right, the percent number of cells with positive fluorescence is shown as a function of the log of Galaxy antibody concentration. All of the FGFR2 antibodies analyzed specifically bound FGFR2 (FIG. 13).

Accordingly, any of antibodies A-F are used to generate biparatopic antibodies (FIGS. 11A-11C) that bind epitopes present in the extracellular domain of FGFR2. Polynucleotides encoding such antibodies can be expressed recombinantly in a desired cultured cell type, and the antibodies purified from the culture. In one embodiment, a culture of HEK cells is co-transfected with four chains encoding a biparatopic antibody, which results in the correct assembly of a biparatopic antibody. In an embodiment, purification of the antibodies may be carried out by nickel purification, for example, using a HisTrap Excel Nickel column. As shown in FIG. 11D, for homodimeric K409R antibodies and heterodimeric F405L/K409R duobodies, using nickel purification, any untagged homodimeric K409R (parental antibody, untagged) antibodies did not bind to the nickel column using an imidazole gradient from 0 to 400 nM over 20 column volumes. As shown in FIG. 11D, heterodimeric F405L/K409R duobodies containing one His-tagged heavy chain, eluted first from the nickel column (top elution profile trace, “His/His Parental Ab”), while the homodimeric F405L antibody, which contained two His tags, showed a later elution time (bottom elution profile trace. “His/No His Biparatopic Ab”). Fractions containing the heterodimeric duobody antibody were pooled, buffer exchanged into PBS and were concentrated to a concentration of 1 mg/ml.

### Example 5: Development of a NANOBIT® Assay to Measure FGFR2 Dimerization

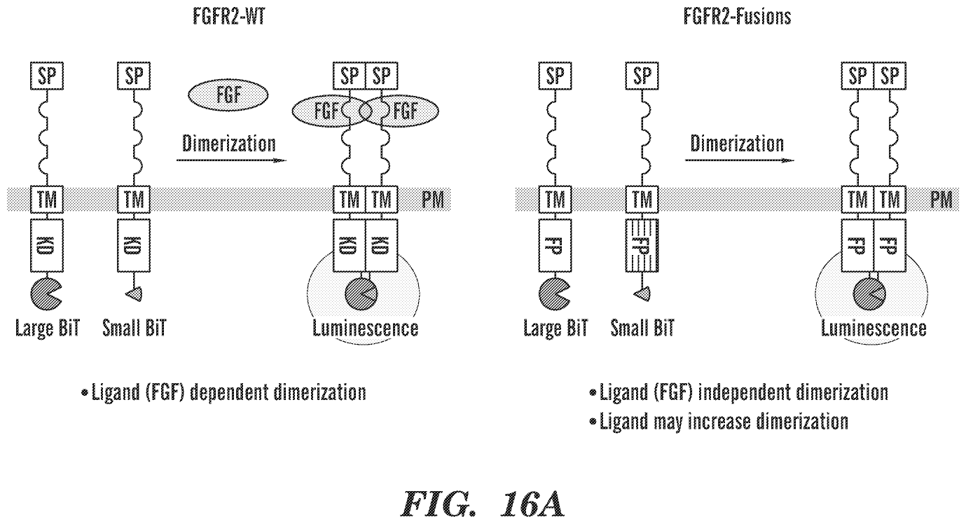

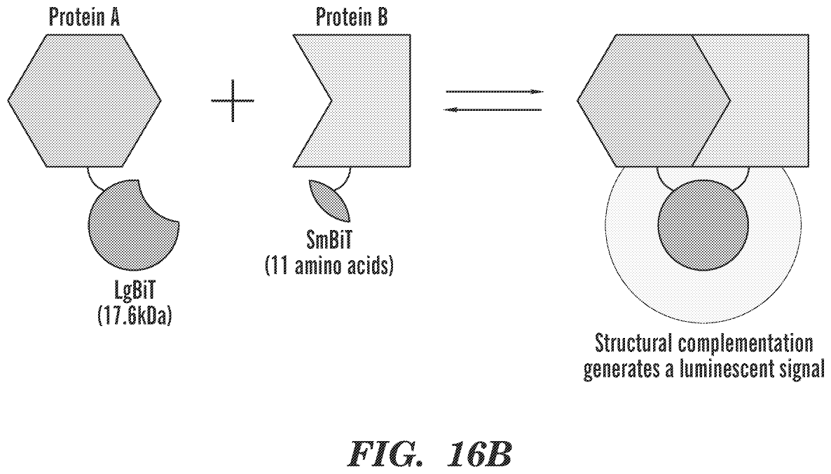

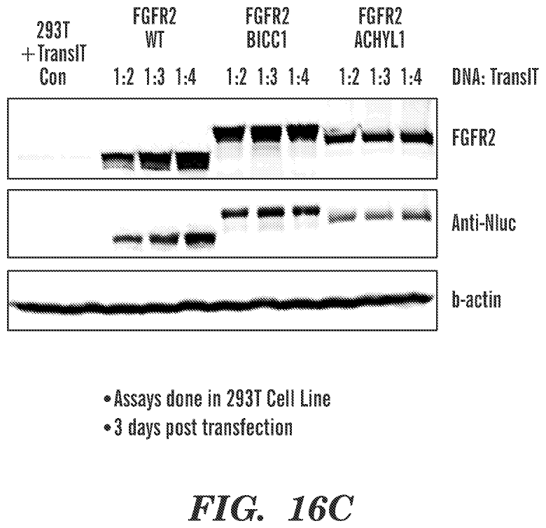

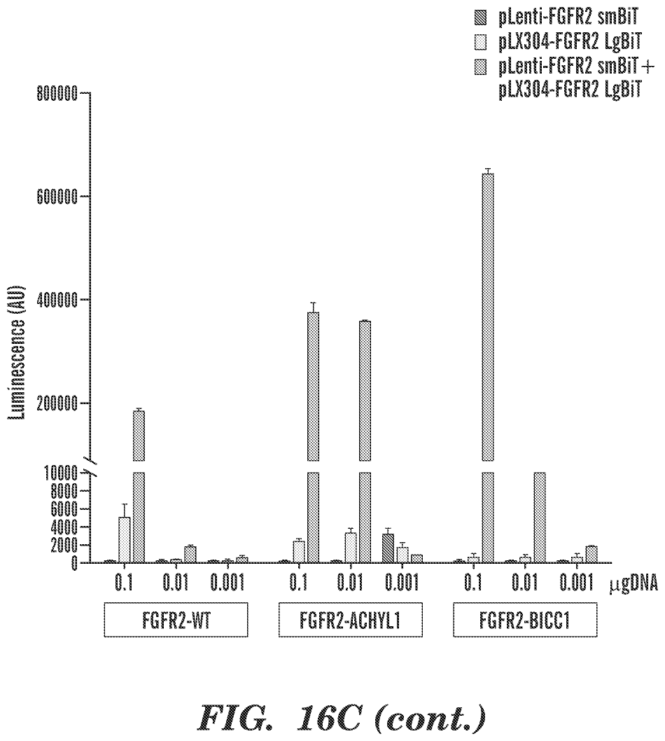

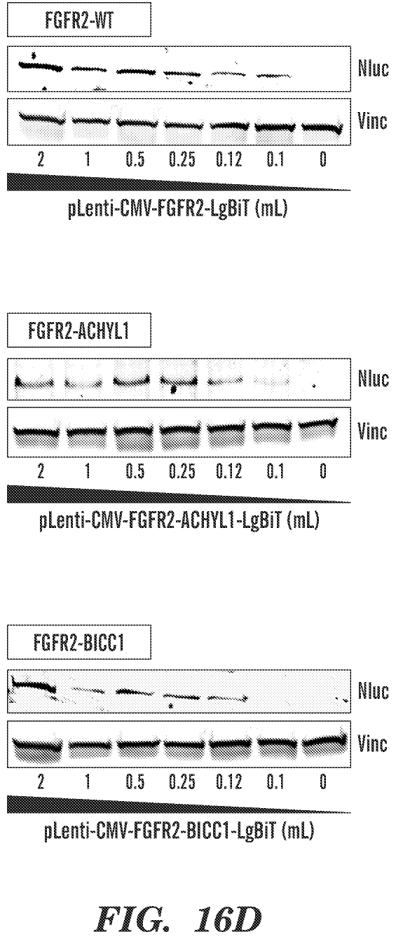

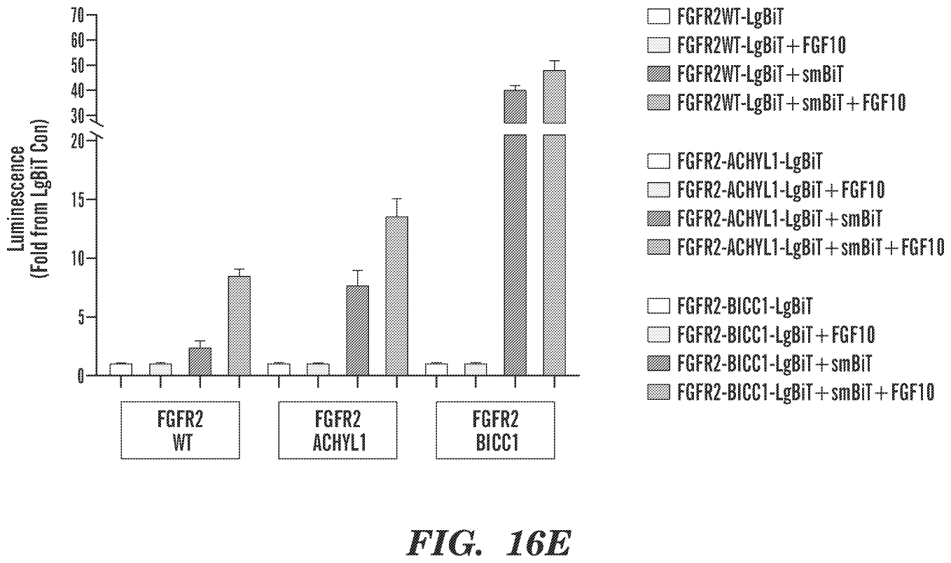

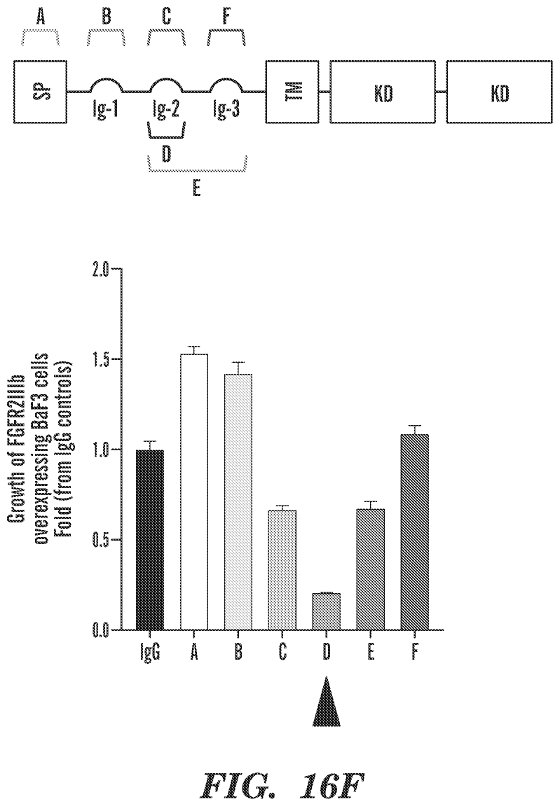

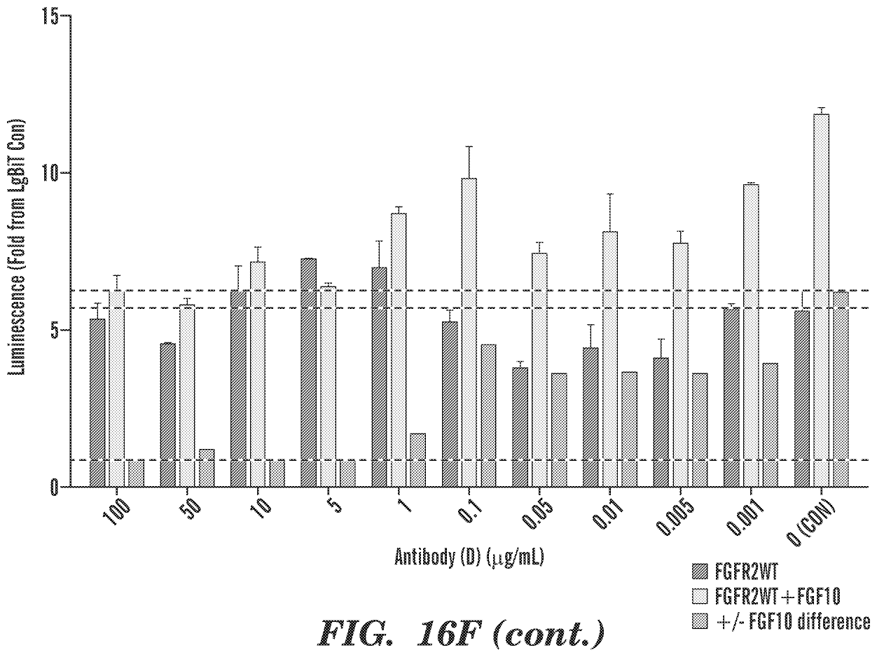

A NANOBIT® assay was developed to measure FGFR2 dimerization in cells, and in particular to screen for biparatopic antibodies that were able to disrupt FGFR2 dimerization. FGFR2 fusions found in patients with CCA facilitate FGFR2 dimerization in cells, which, in turn, activates constitutive FGFR2 signaling, resulting in oncogenic transformation of the cells and increased cell growth and proliferation of cancer cells. NANOBIT® is a two-subunit system based on NanoLuc luciferase that can be used for intracellular detection of protein:protein interactions. (See, e.g., Promega Corporation, Madison, W I, 2017, “Using NANOBIT® Technology to Study the Dynamics of Protein Interactions in Live Cells”) is composed of Large BiT (LgBiT; 17.6 kDa) and Small BiT (SmBiT; 11 amino acids) subunits that are fused to proteins of interest (FIG. 16B). The protein interactions bring the subunits into close proximity to form a functional enzyme that generates a bright, luminescent signal. Lg and sm subunits are selected based on reduced affinity for spontaneous association. In the case of the FGFR2 receptor, FGF10 ligand or FGFR2 fusions bring together the FGFR2 receptors, resulting in an increased luminescence signal in the NANOBIT® assay.

Different expression constructs (e.g., up to eight) were made encoding LgBiT and SmBiT fused to the N and C termini of protein:protein pairs. The constructs were used to transiently or stably transfect cells. NANOBIT® constructs of FGFR2-WT (wild type), FGFR2-ACHYL1 and FGFR2-BICC1 were used for transient expression in HEK293T cells (FIG. 16C). NANOBIT® constructs of FGFR2-WT (wild type), FGFR2-ACHYL1 and FGFR2-BICC1 were used for stable expression in HEK293T cells. (FIGS. 16D and 16E). FIG. 16F shows the results of subjecting the FGFR2 expressing stable cells lines to Antibodies A-F (as identified in FIG. 7B) and performing the NANOBIT® assay. As shown in the graph on the left of FIG. 16F, some of the antibodies inhibited the growth of HEK293T cells stably overexpressing FGFR2 (FGFR2IIIb). In particular, Antibody D, which demonstrated the most pronounced effect on cell growth inhibition compared with the other antibodies, was used in the NANOBIT® assay (graph on the right of FIG. 16F). As shown in this graph, adding Antibody D at increasing concentrations to cells stably expressing FGFR2 in the assay blocks FGF-induced dimerization of the FGFR2 receptor. In the rightmost graph in FIG. 16F, the fold luminescence resulting from FGFR2 WT (wild type) in the assay is represented by the left bar in each set of three bars; the fold luminescence resulting from FGFR2+FGF10 ligand in the assay is represented by the middle bar in each set of three bars; and the fold luminescence resulting from +/−FGF10 ligand difference in the assay is represented by the right bar in each set of three bars shown in FIG. 16F.

### Example 6: Biparatopic Antibody Binding Affinities

Surface Plasmon Resonance (SPR) as known in the art was used to determine the binding affinities of biparatopic antibodies. The SPR-based binding method involves immobilization of a ligand (antibody) on the surface of a sensor chip. The binding partner of interest or an analyte (FGFR2 ECD) flow through the flow channel. Different concentrations of an analyte flow over the ligand, and the interactions of ligand-analyte can be characterized. The SPR signal originates from changes in the refractive index of the light source at the surface of the sensor chip. The increase in mass associated with a binding event causes a proportional increase in the refractive index, which is observed as a change in response-resonance signal. In brief, for the experiments using the antibodies described herein, the SPR assay was carried out by immobilizing antibodies (used as analyte) at a concentration ranging from 1-1000 nM and flow-through FGFR2b alpha IIIb antigen (used as ligand). The antibody kinetic data for the interaction with FGFR2b alpha IIb antigen were fitted to the 2-state and 1-1 binding models using Biocore software. The mean and standard deviation K_{D} values are derived from at least three independent runs.

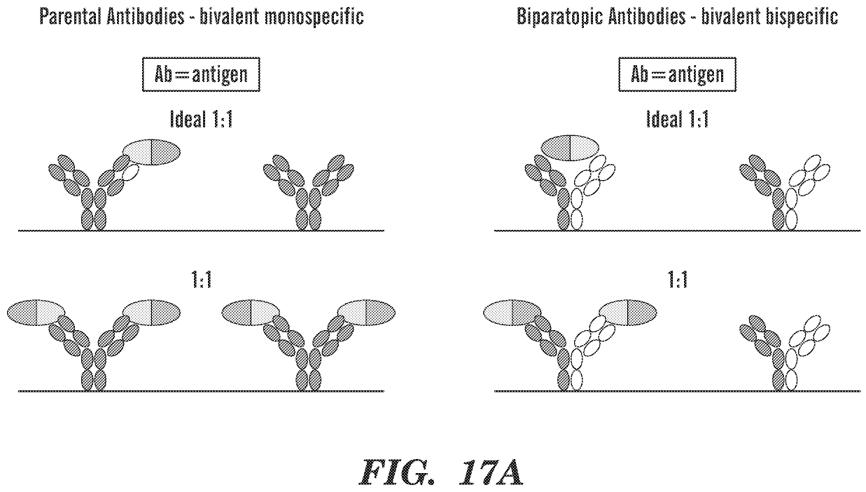

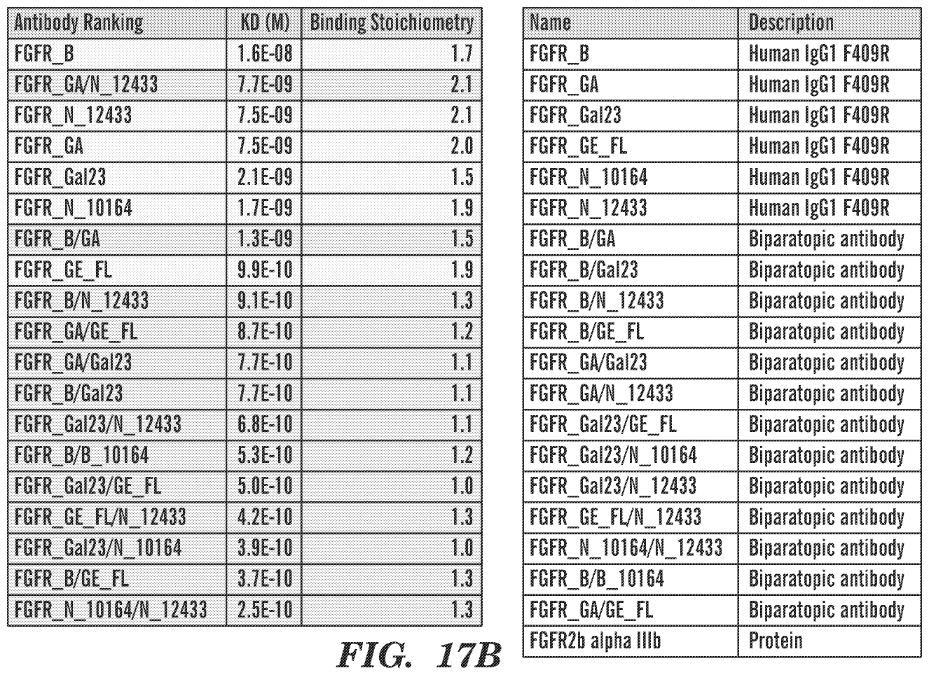

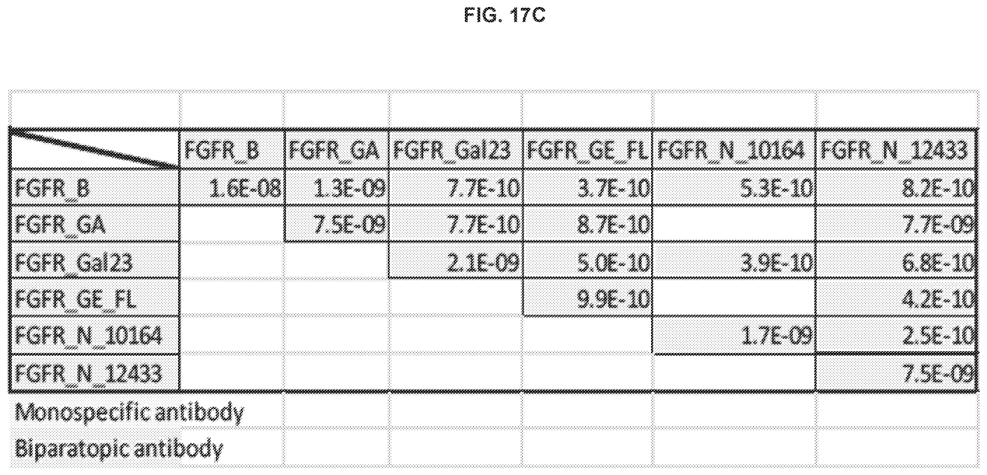

Using SPR, the kinetics of 6 monospecific and 13 biparatopic antibodies for binding to a single antigen, FGFR2b alpha IIb, was measured so as to compare avidity of the biparatopic antibodies to their parental antibodies. Without wishing to be bound by theory, it was expected that the biparatopic antibodies would bind to the FGFR2 antigen more tightly than their parental antibodies and that the tighter binding biparatopic antibodies among the antibodies that were assessed would be more efficient in blocking FGFR2 dimerization (FIG. 17A), thereby leading to inhibition or blocking of constitutive FGFR2 signaling and/or oncogenic transformation. The results of the binding kinetics evaluation are presented in FIGS. 17B and 17C.

The table in FIG. 17B shows the kinetics measurements for 6 monospecific and 13 biparatopic antibodies to bind to a single FGFR2 antigen, FGFR2b alpha IIIb. A comparison of the KD values of all the antibodies binding to FGFR2b alpha IIIb is shown in FIG. 17B, with ranking of the antibodies based on KD values (lowest affinity binders to highest affinity binders). Generally, the biparatopic antibodies bound more tightly to the FGFR2b alpha IIIb antigen than did the monospecific antibodies, except for the FGFR_GA/N_12433 and FGFR_B/GA bispecific antibodies (FIG. 17B). Both FGFR_GA/N_12433 and FGFR_B/GA displayed binding stoichiometries markedly greater than expected for biparatopic antibodies (i.e., binding stoichiometry close to 1). Without intending to be bound by theory, the binding result for the particular biparatopic antibodies suggests that they may not be binding via a conventional engineered biparatopic mechanism, but instead may exhibit binding characteristics similar to those of a monospecific antibody, or a mixture of binding mechanisms.

The parental (monospecific) antibodies exhibited lower binding affinities for FGFR2b alpha IIIb as target antigen compared with the binding affinities exhibited by the biparatopic antibodies. The 6 monospecific antibodies having lower binding affinities for FGFR2 include antibodies FGFR_B (KD (M) 1.6E-08); FGFR_N_12433 (KD (M) 7.5E-09; (FGFR_GA (KD (M) 7.5E-09); FGFR_Gal23 (KD (M) 2.1E-09); FGFR_N_10164 (KD (M) 1.7E-09); and FGFR_GE_FL (KD (M) 9.9E-10), based on the nomenclature in FIG. 7B. The remaining 13 antibodies in the table of FIG. 17B represent biparatopic antibodies having higher binding affinities for FGFR2 alpha IIIb as determined by SPR. The matrix in FIG. 17C provides a comparison of the derived KD for each arm of the biparatopic antibodies.

### Example 7: Impact of Biparatopic Antibodies on FGFR2-Fusion Driven Cell Growth

The impact of biparatopic antibodies on FGFR2-fusion driven cell growth was assessed in a cell-based assay using biparatopic antibodies that bind to the ECD of FGFR2 and generated as described supra (Example 4). As described herein, the biparatopic antibodies were added to cultures of cells (e.g., NIH3T3 cells or BaF3 cells) molecularly engineered to overexpress FGFR2 fusion.

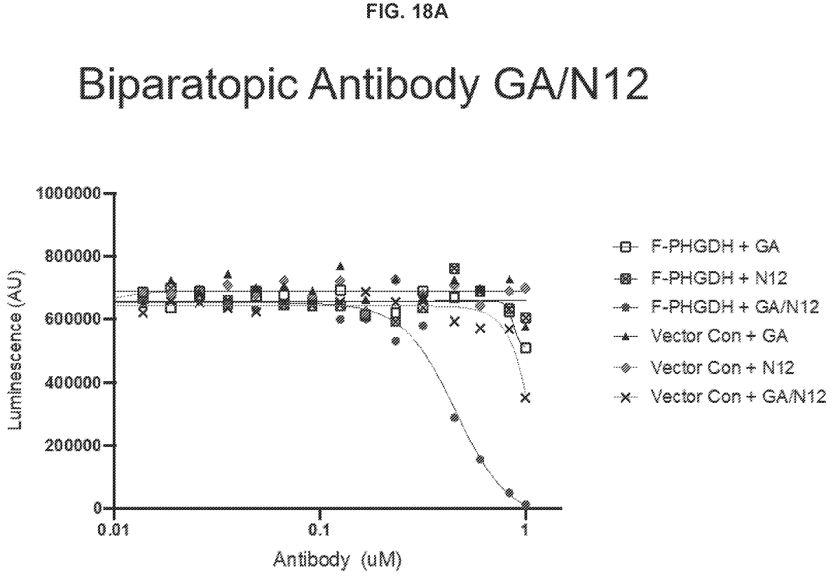

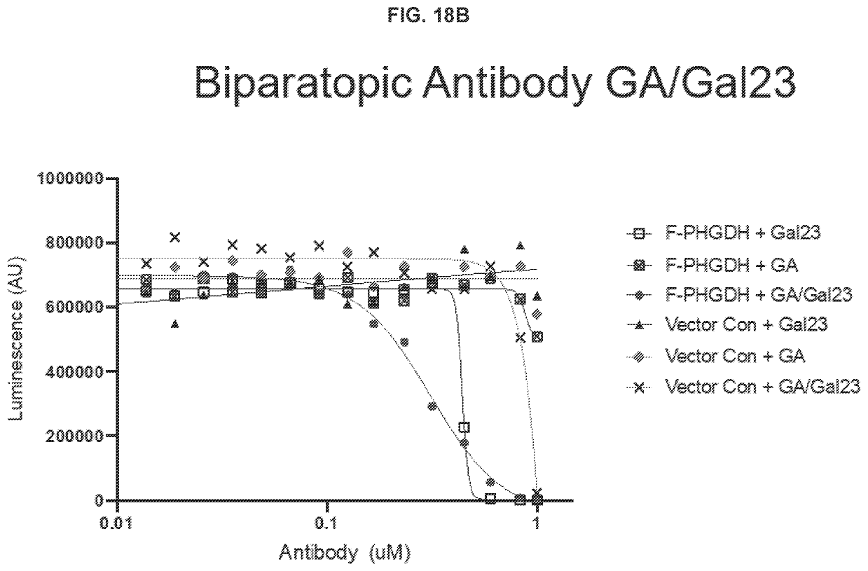

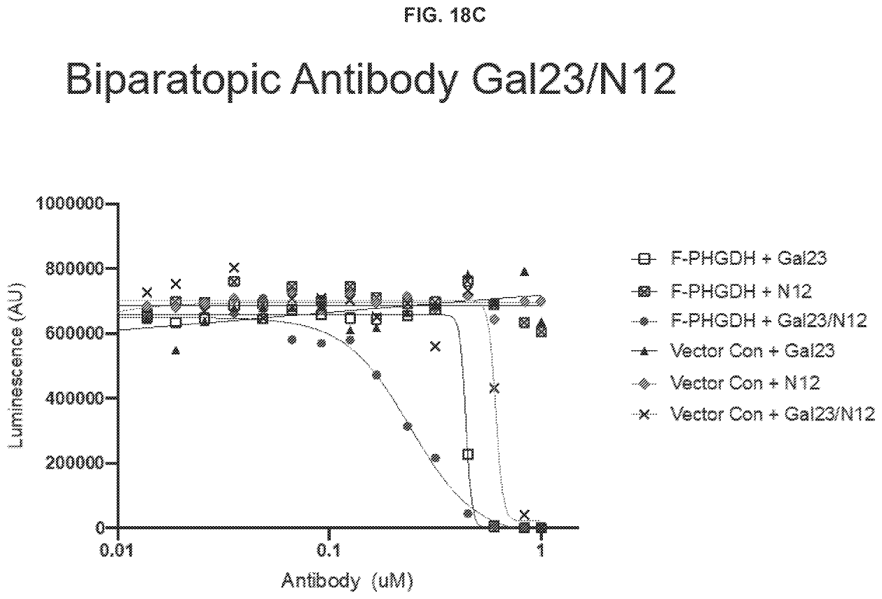

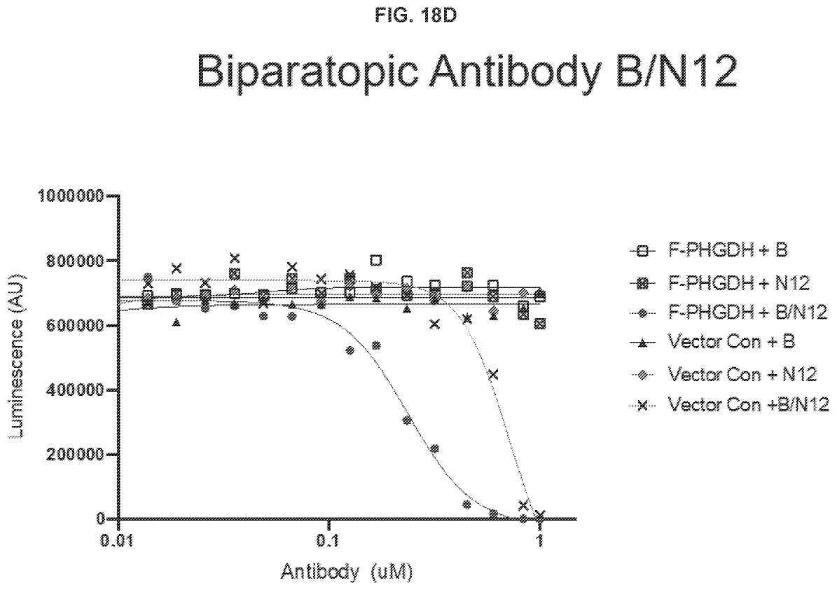

In particular, for assays using BaF3 cells (FIGS. 18A-18D), BaF3 cells overexpressing empty vector control or FGFR2-PHGDH fusion construct were plated in the absence of IL3 and FGF in 384 black well plates at a concentration of 750 cells/well overnight. 6 parental antibodies and 13 biparatopic antibodies were added to each well in duplicates at various concentrations ranging from 1p M to 0.013 μM (1 μM, 0.833 μM, 0.600 μM, 0.450 μM, 0.316 μM, 0.233 μM, 0.166 μM, 0.125 μM, 0.091 μM, 0.066 μM, 0.048 μM, 0.035 μM, 0.025 μM, 0.018 μM, and 0.013 μM). The cell survival was measured using CellTiter-Glo at 5 days post treatment and the IC50 curves were generated using non-linear fit in PRISM 9 software. FGFR2 ECD-binding biparatopic antibodies, namely, Biparatopic antibody GA/N12 (FIG. 18A), Biparatopic antibody GA/Gal23 (FIG. 18B), Biparatopic antibody Gal23/N12 (FIG. 18C) and Biparatopic antibody B/N12 (FIG. 18D), which comprised the binding regions of the antibodies characterized in FIG. 7B, were used in the BaF3 cell-based assay to evaluate its activity and effect of each biparatopic antibody on inhibiting growth of BaF3 cells overexpressing FGFR2-PHGDH. As shown in FIGS. 18A-18D, biparatopic antibodies GA/N12, GA/Gal23, Gal23/N12, and B/N12 were more effective and potent at inhibiting the growth of BaF3 cells molecularly engineered to overexpress FGFR2-PHGDH fusion (found in cholangiocarcinoma patients) compared to control cells overexpressing empty vector. These biparatopic antibodies were also more efficient at inhibiting growth of BaF3 cells overexpressing FGFR2-PHGDH fusion compared to their parental antibodies.

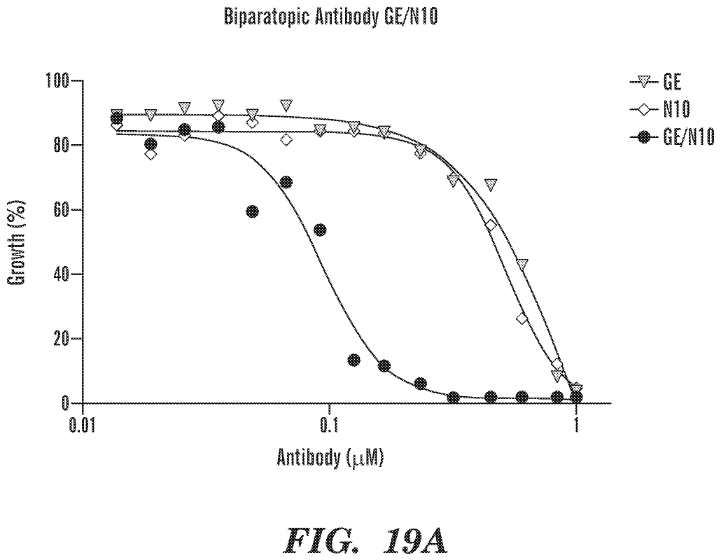

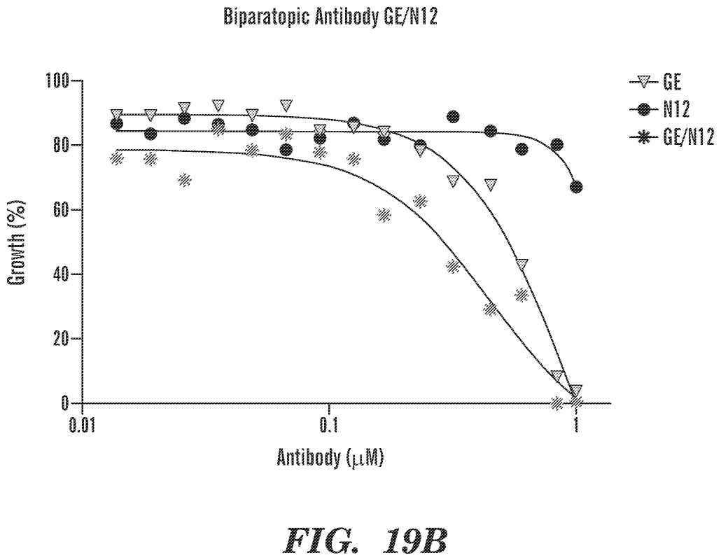

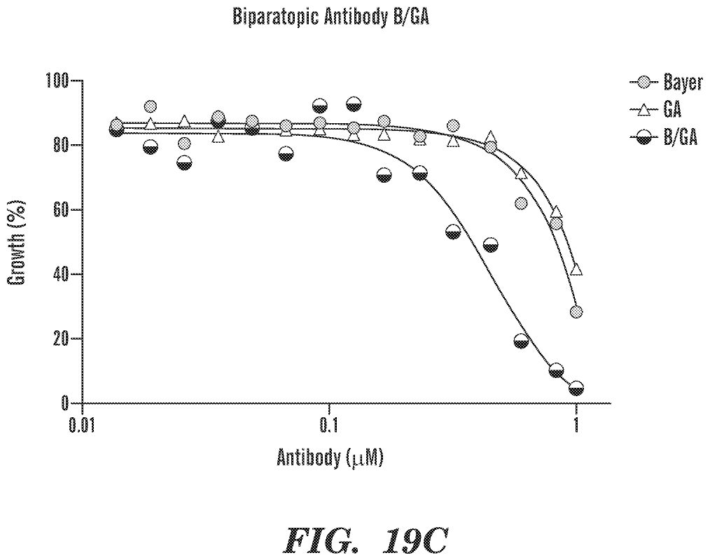

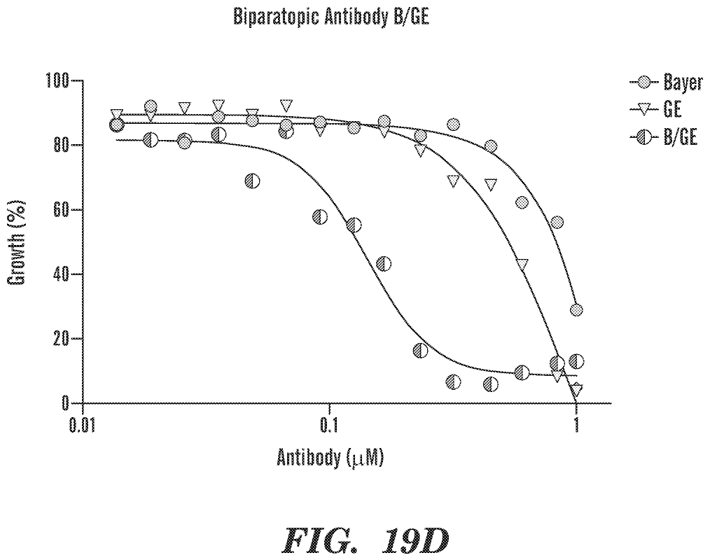

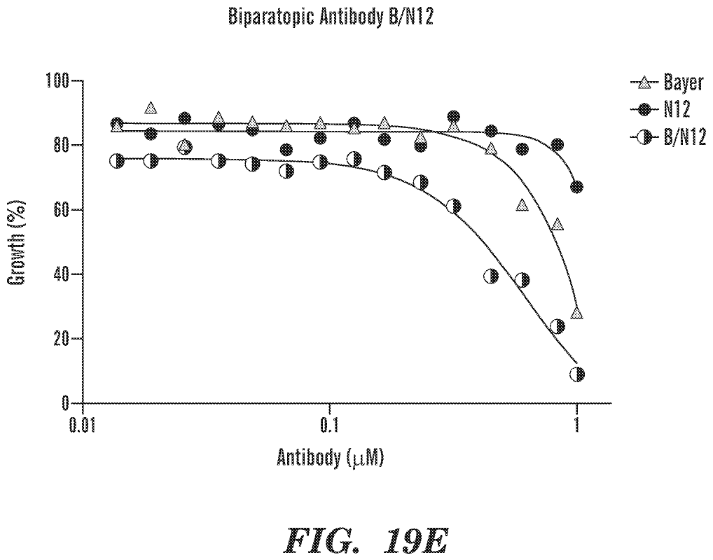

For assays using NIH3T3 cells, (FIGS. 19A-19E), NIH3T3 cells overexpressing a FGFR2-BICC1 fusion construct were plated in 384 black well plates at a concentration of 1000 cells/well overnight. 6 parental antibodies and 13 biparatopic antibodies were added to each well in duplicates at various concentrations ranging from 1 μM to 0.013 μM (1 μM, 0.833 μM, 0.600 μM, 0.450 μM, 0.316 μM, 0.233 μM, 0.166 μM, 0.125 μM, 0.091 μM, 0.066 μM, 0.048 μM, 0.035 μM, 0.025 μM, 0.018 μM, and 0.013 μM). The cell confluency was measured using Incucyte at 60 hours post treatment, and the IC50 curves were generated using non-linear fit in PRISM 9 software. As shown in FIGS. 19A-19E, biparatopic antibodies: GE/N10, GE/N12, B/GE, B/GA, and B/N12 were more effective and potent at inhibiting growth of NIH3T3 cells overexpressing FGFR2-BICC1 fusion (found in cholangiocarcinoma patients) compared to their parental antibodies.

The 6 parental antibodies as described above included Antibody A (B), Antibody B(Gal23), Antibody C(N10), Antibody D (GE), Antibody E (GA), Antibody F (N12), e.g., as presented in FIG. 7B. Biparatopic antibodies are the pair-wise combinations of the six (6) parental antibodies. Thirteen (13) biparatopic antibodies were successfully generated via duobody reactions. (See, e.g., FIG. 17B, which presents the biparatopic antibodies and their KDs for binding FGFR2).

### Other Embodiments

From the foregoing description, it will be apparent that variations and modifications may be made to some aspects and embodiments herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A polypeptide that specifically binds two epitopes in the extracellular domain of a fibroblast growth factor receptor 2 (FGFR2), wherein the polypeptide comprises two antigen binding fragments of anti-FGFR2 antibodies.

2. The polypeptide of claim 1, wherein the anti-FGFR2 antibodies are selected from the group consisting of M048-D01, GAL-FR23, 10164, 2B 1.3.12, GAL-FR21, and 12433.

3. A biparatopic antibody that specifically binds two epitopes in the extracellular domain of a fibroblast growth factor receptor 2 (FGFR2), wherein the biparatopic antibody comprises antigen binding fragments of an antibody selected from the group consisting of M048-D01, GAL-FR23, 10164, 2B 1.3.12, GAL-FR21, and 12433.

4. The polypeptide of claim 1, wherein the polypeptide or antibody comprises one or more complementarity determining regions of the antibody.

5. The polypeptide of claim 1, wherein the polypeptide or antibody comprises a heavy chain variable domain (VH) or a light chain variable domain (VL).

6-9. (canceled)

10. The polypeptide of claim 1, wherein antibody or polypeptide binding blocks ligand binding to FGFR2 and/or reduces FGFR2 activity.

11. (canceled)

12. The polypeptide of claim 1, wherein the antigen binding fragment has at least 85%, 90%, 95%, comprises, or consists essentially of amino acid sequence identity to the sequence of M048-D01, GAL-FR23, 10164, 2B 1.3.12, GAL-FR21, or 12433.

13-15. (canceled)

16. The polypeptide of claim 1, wherein the polypeptide comprises an affinity tag or a detectable amino acid sequence.

17. (canceled)

18. The biparatopic antibody of claim 3, wherein the biparatopic antibody comprises FGFR2 antigen binding fragments of antibody M048-D01 and antibody 12433; or antigen binding fragments of antibody GAL-FR21 and antibody 12433; or antigen binding fragments of HuGAL-FR21 and antibody 12433; or antigen binding fragments of antibody HuGAL-FR21 and antibody GAL-FR23; or antigen binding fragments of antibody GAL-FR23 and antibody 12433; or antigen binding fragments of antibody M048-D01 and antibody 12433; or antigen binding fragments of antibody 2B 1.3.12 and antibody 10164; or antigen binding fragments of antibody 2B 1.3.12 and antibody 12433; or antigen binding fragments of antibody GAL-FR23 and antibody 2B 1.3.12; or antigen binding fragments of antibody GAL-FR23 and antibody HuGAL-FR21; or antigen binding fragments of antibody GAL-FR23 and antibody 12433.

19. The polypeptide of claim 1, wherein the polypeptide or the biparatopic antibody has a KD for binding to FGFR2 of from about 7.7E-09 to about 9.1E-10.

20. (canceled)

21. A method of inhibiting the proliferation or reducing survival of a neoplastic cell, the method comprising contacting the cell with an effective amount of the polypeptide or antibody of claim 1, thereby inhibiting proliferation or reducing viability.

22. The method of claim 21, wherein the polypeptide or antibody induces cell death of the neoplastic cell.

23-25. (canceled)

26. A method of treating cancer in a subject, the method comprising administering to the subject an effective amount of the polypeptide or antibody of claim 1, thereby treating the cancer.

27. (canceled)

28. A method of treating cholangiocarcinoma in a subject, the method comprising administering to the subject an effective amount of a biparatopic antibody comprising antigen binding fragments of an antibody selected from the group consisting of M048-D01, GAL-FR23, 10164, 2B 1.3.12, GAL-FR21, or 12433.

29. The method of claim 28, wherein the method comprises administering to the subject an effective amount of a biparatopic antibody comprising FGFR2 antigen binding fragments of antibody M048-D01 and antibody 12433; or antigen binding fragments of antibody GAL-FR21 and antibody 12433; or antigen binding fragments of HuGAL-FR21 and antibody 12433; or antigen binding fragments of antibody HuGAL-FR21 and antibody GAL-FR23; or antigen binding fragments of antibody GAL-FR23 and antibody 12433; or antigen binding fragments of antibody M048-D01 and antibody 12433; or antigen binding fragments of antibody 2B 1.3.12 and antibody 10164; or antigen binding fragments of antibody 2B 1.3.12 and antibody 12433; or antigen binding fragments of antibody GAL-FR23 and antibody 2B 1.3.12; or antigen binding fragments of antibody GAL-FR23 and antibody HuGAL-FR21; or antigen binding fragments of antibody GAL-FR23 and antibody 12433.

30. (canceled)

31. An isolated nucleic acid molecule that encodes the polypeptide or antibody of claim 1.

32. A vector comprising a nucleic acid molecule that encodes the polypeptide or antibody of claim 1.

33-35. (canceled)

36. A host cell comprising the vector of claim 32.

37. A pharmaceutical composition comprising an effective amount of the polypeptide or antibody of claim 1, or fragments thereof, in a pharmaceutically acceptable excipient.

38. A method of treating cholangiocarcinoma in a subject, the method comprising administering to the subject an effective amount of an antibody of claim 1 and an effective amount of pemigatinib or NVP-BGJ398.

39. The biparatopic antibody of claim 18, wherein the biparatopic antibody comprising FGFR2 antigen binding fragments of antibody HuGAL-FR21 and antibody 12433; or antigen binding fragments of antibody HuGAL-FR21 and antibody GAL-FR23; or antigen binding fragments of antibody GAL-FR21 and antibody 12433; or antigen binding fragments of antibody GAL-FR23 and antibody 12433 inhibited growth of cells expressing an FGFR2 fusion.

40. The biparatopic antibody of claim 39, wherein the FGFR2 fusion is FGFR2-PHGDH or FGFR2-BICC1.

41. The biparatopic antibody of claim 18, wherein the biparatopic antibody comprising FGFR2 antigen binding fragments of antibody 2B 1.3.12 and antibody 10164; or antigen binding fragments of antibody 2B 1.3.12 and antibody 12433; or antigen binding fragments of antibody GAL-FR23 and antibody 2B 1.3.12; or antigen binding fragments of antibody GAL-FR23 and antibody HuGAL-FR21; or antigen binding fragments of antibody GAL-FR23 and antibody 12433 inhibited growth of cells expressing an FGFR2 fusion.

42-44. (canceled)
